SEQUENCE LISTING (15 sequences)

SEQ ID NO: 1 - DNA, 52 bp, homo sapiens
ctgaaccagggagctccccgaaggcgygaaccagggttgaattgcactccgc

SEQ ID NO: 2 - DNA, 52 bp, homo sapiens
ctgagagaagtcaaattcctagaaacrgacgtgtctaaatatttgccggggt

SEQ ID NO: 3 - DNA, 52 bp, homo sapiens
cttttgttttcctttctgtgagcaatktcacccaaattggaaccatgctgta

SEQ ID NO: 4 - DNA, 52 bp, homo sapiens
agaacaaatgctgtatgattccccctmcatgaggtgctgagagaagtcaaat

SEQ ID NO: 5 - DNA, 52 bp, homo sapiens
tattcatttttccaacaagcatcctgmcccaggtcgctctgtctgtctcaat

SEQ ID NO: 6 - DNA, 52 bp, homo sapiens
cctaaatatgatttcctaaatcatacrgacatatttccttgggagctataca

SEQ ID NO: 7 - DNA, 52 bp, homo sapiens
tcatataacaatatgaaagccagagayagctcgtctgagacacagatgaaca

SEQ ID NO: 8 - DNA, 52 bp, homo sapiens
tcctggggaagaggcgggagcggcacytgcagtccttcagcagaagcgactc

SEQ ID NO: 9 - DNA, 52 bp, homo sapiens
cagagagaaagggagctgagggaatgsagaggctgcccactgagggcagggg

SEQ ID NO: 10 - DNA, 52 bp, homo sapiens
ctgagctccatggggcagcttttatcyctgacagaagggcagtcccagctga

SEQ ID NO: 11 - DNA, 52 bp, homo sapiens
tagcgactgggtgacaataaattaagmcaagtggctaatttataaataaaat

SEQ ID NO: 12 - DNA, 52 bp, homo sapiens
ctgtggagcacagaactgccaggaacyagggcccctggatgactgagtgggg

SEQ ID NO: 13 - DNA, 52 bp, homo sapiens
cttcctgacatcactccaatgtcctgyttctgtggttacatcttccgctaat

SEQ ID NO: 14 - DNA, 52 bp, homo sapiens
agagggcacagccagtgtggtcaggtrggagcagagggaaggggtagcaggt

SEQ ID NO: 15 - DNA, 52 bp, homo sapiens
catggtctcagtctgtagcccaagctrgagcatagtagtggcacaatcgcca

(19) United States
(12) Patent Application Publication
(10) Pub. No.: US 2011/0027229 A1
(43) Pub. Date: Feb. 3, 2011

(54) CONTINUOUS SUBCUTANEOUS ADMINISTRATION OF INTERFERON-ALPHA TO HEPATITIS C INFECTED PATIENTS

(75) Inventors: William P. Van Antwerp, Valencia, CA (US); Eric A. Grovender, Minneapolis, MN (US); Harry L. A. Janssen, Rotterdam (NL); Robert J. De Knegt, Rotterdam (NL)

Correspondence Address: GATES & COOPER LLP;HOWARD HUGHES CENTER, 6701 CENTER DRIVE WEST, SUITE 1050, LOS ANGELES, CA, 90045, US

(73) Assignee: MEDTRONIC, INC., Minneapolis, MN (US)

(21) Appl. No.: 12/848,856

(22) Filed: Aug. 2, 2010

Related U.S. Application Data
(60) Provisional application No. 61/230,488, filed on Jul. 31, 2009.

(51) Int. Cl. A61K 38/21; C07K 14/52; A61P 31/14; A61M 5/142

(52) U.S. Cl. 424/85.7; 530/351; 604/151; 604/66

(57) ABSTRACT

Methods and systems for treating Hepatitis C infections are provided. Typically the method comprises administering interferon-α to the patient subcutaneously using a continuous infusion apparatus, wherein this therapeutic regimen is sufficient to maintain circulating levels of interferon-α in the serum of the patient above a target concentration for a certain period of time.

### CROSS REFERENCE TO RELATED APPLICATIONS

This application claims priority under Section 119(e) from U.S. Provisional Application Ser. No. 61/230,488 filed Jul. 31, 2009, the contents of which are incorporated herein by reference. This application is related to International Application Numbers PCT/US2009/038617 (International Publication No. WO 2009/120991); PCT/US2009/060121 (International Publication No. WO 2010/047974); and PCT/US2008/078843 (International Publication No. WO 2009/046369), the contents of which are incorporated by reference.

### FIELD OF THE INVENTION

This invention relates to therapies involving the administration of interferon-α for the treatment of pathological conditions (e.g. Hepatitis C virus infections). In particular, this invention relates to methods and systems for administering interferon-α in a manner that controls the in vivo levels of interferon-α in the patient in order to optimize the outcome of a therapeutic regimen(s).

### BACKGROUND OF THE INVENTION

It is estimated that Hepatitis C has infected nearly 20 million people worldwide, and infects 3-4 million people per year (see e.g., Hepatitis C, WHO; Hepatitis C Infection, The National Institute on Drug Abuse (NIDA)). Hepatitis C virus (HCV) infection is the most common chronic blood borne infection in the United States. It accounts for about 15 percent of acute viral hepatitis, 60 to 70 percent of chronic hepatitis, and up to 50 percent of cirrhosis, end-stage liver disease, and liver cancer. Of the U.S. population, 1.6 percent, or an estimated 4.1 million Americans, have antibody to HCV (anti-HCV), indicating ongoing or previous infection with the virus. Hepatitis C causes an estimated 10,000 to 12,000 deaths annually in the United States. Moreover, chronic liver disease is the tenth leading cause of death among adults in the United States, accounting for approximately 25,000 deaths annually, or approximately 1% of all deaths. The high prevalence of chronic HCV infection has important public health implications for the future burden of chronic liver disease in the United States. Data derived from the National Health and Nutrition Examination Survey (NHANES III) indicates that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million.

Currently, treatments for chronic hepatitis C infection typically include the administration of combinations of ribavirin and interferon-α. Ribavirin is a nucleoside analog that when incorporated into cells, interferes with viral replication (similar to action of AZT in HIV infection). It is interesting to note that while ribavirin is not effective as a stand-alone therapy for HCV, it potentiates interferon-α effectiveness through an as yet unknown mechanism. For example, in controlled clinical studies, ribavirin monotherapy has negligible efficacy and PEG-interferon-α alone has an effectiveness of 11% in a genotype 1 population. However, when ribavirin is combined with interferon-α, the therapeutic effectiveness of the combination is 29% in this population (see, e.g. Sjogren et al., Dig Dis Sci. 2005 April; 50(4):727-32). A variety of such therapeutic methods for the treatment of hepatitis C infection are described for example in PCT patent applications such as WO 2005/067454; WO2005/018330; WO2005/062949; WO2006/130553; WO20060130626; and WO2006/130627; United States patent applications such as 2005/0191275; US 2005/0201980; 2007/004635; US2006/281689; and 2006/276405 and articles such as Perdita, et. al., World Journal of Gastroenerology, 7(2):222-227, (April 2001); Bizollon, et. al., Hepatology, 26(2):500-504, (August 1997); Alberti, et. al. Liver Transplantation, 7(10):870-876, (October 2001); Shakil, et. al., Hepatology, 36(5):1253-1258, (November 2002); Schalm, et. al., Gut, 46:562-568, (April 2000); and Yurdaydin et al., Journal of Viral Hepatitis, 12(7):262-268, (May 2005). Unfortunately however, while great strides have been made in the treatment of HCV infection, clinical success rates are only about 50% and have progressed slowly since the introduction of interferon-α into the clinic (see, e.g. Smith, R., Nat Rev Drug Discov. 2006, 5(9):715-6).

As current clinical practices eliminate HCV in only about 50% of infected individuals, new therapies are highly desirable. The development of such therapies is complicated however by the observation that host factors such as ethnicity, obesity, insulin resistance and hepatic fibrosis, as well as viral factors such as genotype and baseline viral load, can have a profound impact on the success of a given therapeutic regimen. In addition, current therapeutic regimens last for an extended period of time and patients often suffer from a host of adverse dose-dependent side-effects including severe flu-like symptoms, which can negatively impact patient compliance and outcome. Accordingly, there is a need for improved methods for treating viral infections such as hepatitis C, in particular the development of methods and systems for administering interferon-α in a manner that optimizes the response to, and outcome of, therapies comprising this agent.

### SUMMARY OF THE INVENTION

The disclosure provided herein includes results obtained from a clinical trial designed to study the continuous subcutaneous administration of interferon-α combined with ribavirin in chronic hepatitis C treatment experienced patients. Clinical data obtained from this trial shows that the continuous subcutaneous administration of interferon-α can be used to maintain in vivo concentrations of interferon-α above a critical efficacy threshold for an extended period of time. The clinical data further shows that therapeutic regimens following the methodologies disclosed herein can be used, for example, to eliminate hepatitis C virus in patients observed to be refractory to conventional antiviral therapy.

The invention disclosed herein has a number of embodiments that relate to therapeutic regimens for the treatment of hepatitis C infections. One illustrative embodiment of the invention is a method of administering interferon-α to a patient infected with hepatitis C virus, the method comprising administering interferon-α to the patient using a continuous infusion apparatus, wherein the interferon-α is administered to the patient using a therapeutic regimen sufficient to maintain circulating levels of the interferon-α in the serum of the patient above a certain steady state concentration for a period of time, for example a concentration of at least 100 picograms per milliliter (pg/mL) for at least 1 week to at least 48 weeks. In typical embodiments of the invention, such therapeutic regimens are sufficient to reduce levels of HCV in the patient by at least 100-fold.

Embodiments of the invention include personalized therapeutic regimens tailored to consider one or more characteristics specific to the patient and/or the virus infecting the patient. For example, the presence or absence of specific single nucleotide polymorphisms on chromosome 19, band 13 can be used to assess the likelihood of HCV viral clearance following a therapeutic regimen comprising interferon-α and ribavirin as well as to predict the speed of the response to these therapeutic agents. Consequently, certain methodological embodiments of the invention comprise the steps of determining a polynucleotide sequence on chromosome 19 in the patient (e.g. See the NCBI Single Nucleotide Polymorphisms database (http://www.ncbi.nlm.nih.gov/projects/SNP/) for one or more of the single nucleotide polymorphisms designated rs12979860, rs12980275, rs8099917, rs12972991, rs8109886, rs4803223, rs12980602, rs8105790, rs11881222, rs8103142, rs28416813, rs4803219, rs8099917 or rs7248668); and then administering interferon-α to a patient infected with hepatitis C virus by a method comprising administering interferon-α to the patient subcutaneously using a continuous infusion apparatus, wherein the interferon-α is administered to the patient using a therapeutic regimen sufficient to maintain circulating levels of the interferon-α in the serum of the patient above a steady state concentration (e.g. at least 100 pg/mL). In certain embodiments of the invention, information on the SNP genotype is used to determine or modulate a parameter of a therapeutic regimen, for example to determine the duration of interferon-α administration (e.g. more than 48 weeks, less than 48 weeks etc.).

Embodiments of the invention also include therapeutic regimens designed to use therapeutic compositions selected to have certain properties (e.g. properties that control the in vivo bioavailability profile of a therapeutic agent within that composition). In one such embodiment of the invention, the interferon-α is not conjugated to a polyol. In some embodiments of the invention, the patient's prior history of therapy is considered, for example by identifying the patient as a relapser or a non-responder prior to initiating the therapeutic regimen. In one illustrative embodiment of the invention, interferon-α2a/2b that is not conjugated to a polyol is administered to a patient identified as a relapser or a non-responder using a therapeutic regimen sufficient to maintain circulating levels of interferon-α in the serum of the patient above a steady state concentration of at least 100-700 pg/mL for at least 1 week to at least 48 weeks. Typically these methods further comprise administering a small molecule inhibitor of viral replication such as ribavirin.

Another illustrative embodiment of the invention that considers one or more characteristics specific to the patient is a method of administering an interferon-α to a patient infected with hepatitis C virus, the method comprising administering a test dose of an interferon-α to the patient and then observing a concentration of circulating interferon-α in the serum of the patient that results from the test dose. In this embodiment, the concentration of circulating interferon-α observed in response to the test dose is then used to design a patient-specific therapeutic regimen, one that comprises administering interferon-α to the patient subcutaneously using a continuous infusion apparatus in an amount sufficient to maintain circulating levels of interferon-α in the serum of the patient above a specific in vivo concentration for a specific period of time, for example above 100 pg/mL for at least 1 week to at least 48 weeks. In related embodiments of the invention, the patient-specific therapeutic regimen is selected to maintain serum interferon-α concentrations in the patient at a value greater than a critical concentration threshold that induces and/or facilitates a patient's sustained response to a therapeutic regimen.

Other embodiments of the invention include systems for administering interferon-α to a patient having a hepatitis C infection. In such embodiments of the invention, the system can comprise for example: a continuous infusion pump having a medication reservoir comprising interferon-α; a processor operably connected to the continuous infusion pump and comprising a set of instructions that causes the continuous infusion pump to administer the interferon-α to the patient according to a therapeutic regimen comprising administering interferon-α to the patient subcutaneously; wherein the therapeutic regimen is sufficient to maintain circulating levels of interferon-α in the serum of the patient above a steady state concentration of at least 100-700 pg/mL. In certain embodiments of the invention, a polynucleotide sequence of the patient using the system for administering interferon-α to a patient is determined, the polynucleotide sequence comprising a single nucleotide polymorphism (SNP) designated rs12979860, rs12980275, rs8099917, rs12972991, rs8109886, rs4803223, rs8103142, rs28416813, rs4803219, rs4803217, rs581930, rs8105790, rs11881222, rs7248668 or rs12980602; and the processor in the system is used to modulate a parameter of the patient-specific therapeutic regimen using determined polynucleotide sequence information, wherein the parameter comprises a duration of interferon-α administration or an interferon-α dose.

In certain embodiments of the invention, the system for administering interferon-α is coupled to an electronic system for managing medical data on an electronic communication network. For example one such electronic system can comprise at least one electronic server connectable for communication on the communication network, the at least one electronic server being configured for: receiving a first physiological parameter observed in a patient (e.g. a patient's viral load) setting a first dose of the interferon-α for infusion by the continuous infusion pump, based on the first physiological parameter; receiving second physiological parameter information of the patient indicative of a response of the patient to the interferon-α of the first dose; and then setting a second dose of the interferon-α for infusion by the continuous infusion pump, based on the second physiological parameter.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE FIGURES

FIGS. 1A and 1B provide analyses of data from HCV infected patients treated with interferon-α following the therapeutic regimens disclosed herein. During the course of the disclosed clinical trial, measurements were taken of pharmacokinetic and pharmacodynamic parameters including mean interferon-α (FIG. 1A) and neopterin (FIG. 1B) concentrations that result from therapeutic regimens disclosed herein (N=23). The data provided in the graphs shown in FIGS. 1A and 1B show that there is a strong dose response observed in patients in response to interferon-α administration following the disclosed therapeutic regimens. The data shown in FIGS. 1A and 1B further show that delivering higher concentrations of interferon-α following the therapeutic regimens disclosed herein leads to correspondingly higher sustained concentrations of interferon-α in vivo.

FIG. 2 provides viral decay analyses from a subset of HCV infected patients that were previously shown to be severely interferon-α resistant and were subsequently treated using the therapeutic regimens disclosed herein. The viral decay curves in the 6 MIU/day treatment group treatment failures are illustrated in the graphic data shown in this Figure. In the 6 MIU/day therapeutic regimen group there were 5 subjects that showed significant resistance. Of these 5 subjects, patient 8 showed a robust response at week 8 with subsequent rebound. In previous therapy, all of these 5 subjects were either therapy failures at week 12 or week 24. Five subjects in this 6 MIU/day therapeutic regimen group with more clinically significant HCV declines are shown in FIG. 3. As with all patients in the trial, subjects in the 6 MIU per day group were previously shown to be severely resistant to conventional HCV therapies using pegylated interferon-α.

FIG. 3 provides viral decay analyses of a subset of HCV infected patients in the 6 MIU per day therapeutic regimen group, robust response group, all of whom were previously shown to be severely resistant to conventional HCV therapies using pegylated interferon-α. In this treatment group, viral decay curves in response to this treatment show a clinically significant response following the therapeutic regimens disclosed herein.

FIGS. 4A and 4B provide viral decay analyses of a subset HCV infected patients in the 9 MIU per day therapeutic regimen group, all of whom were previously shown to be severely resistant to conventional HCV therapies using pegylated interferon-α. The data provided in FIG. 4A shows that there were 4 subjects who remained interferon-α resistant. However, the data provided in FIG. 4B shows that that 6 of the 10 subjects in the 9 MIU per day therapeutic regimen group show a robust response even though these patients were found to be previously resistant to pegylated interferon-α treatment.

FIG. 5 provides viral decay analyses of a subset of HCV infected patients in the 12 MIU per day therapeutic regimen group, all of whom were previously shown to be severely resistant to conventional HCV therapies using pegylated interferon-α. In this 12 MIU/day therapeutic regimen group, there are no interferon-α resistant subjects to the current therapy. Three patients have withdrawn from the trial. 9 patients show a robust response.

FIG. 6 provides viral decay data at the four-week timepoint for the 6, 9, and 12 MIU per day therapeutic regimen groups. As shown by the curves in this graph, at four weeks there is a significant difference in viral load that relates to differences in the doses.

FIG. 7 provides data comparing viral decay by dosing in patient groups receiving the 6, 9 or 12 MIU per day therapeutic regimens. As shown by the data presented in these bar graphs at four weeks there is a significant difference in viral decay observed with different doses of interferon-α.

FIG. 8 provides information on how the serum interferon-α concentrations in vivo that result from the therapeutic regimens disclosed herein influences the viral decay data at the four week timepoint.

FIG. 9A presents an exemplary generalized computer system 202 that can be used to implement elements of the present invention. FIG. 9B presents one embodiment of a specific illustrative computer system embodiment that can be used with embodiments of the invention in the treatment of Hepatitis C virus infection.

FIG. 10 provides a summary of aspects of the SCIN-C clinical trial in a Table format. Regarding superscript numerals 1-8 in this Table, Hematology¹: Hb, platelets, leucocytes, absolute neutrophil count, prothrombin time; Hematology²: Hb, platelets, leucocytes, absolute neutrophil count; Chemistry³: AST, ALT, total bilirubin, GGT, alkaline phosphatase, albumin, creatinine, TSH, LDH, Na, K, urea, amylase, CPK, glucose, ferritin, serum iron, transferrin, transferrin saturation, α-fetoprotein, IgG, ANA, ASMA; Chemistry⁴: AST, ALT, total bilirubin, GGT, alkaline phosphatase, albumin, creatinine, TSH, 2′5′-OAS, β₂-microglobulin; Chemistry⁵: AST, ALT, 2′5′-OAS, β₂-microglobulin; HCV RNA⁶: qualitative assay; HCV RNA⁷: quantitative assay; and HCV RNA⁸: quantitative assay, if negative qualitative assay will also be performed.

FIG. 11A provides a table showing IL28B SNP sequence information for rs12979860, rs12890275, rs4803217, rs8099917 and rs8103142. FIG. 11B provides a table showing a combination of IL28B SNP rs12979860 sequence information, interferon-α dose information, and virological kinetic information obtained from subjects enrolled in the SCIN-C study. As shown in this Table, there were 3 subjects with the CC genotype, 21 subjects with the TC genotype, and 6 subjects with the TT genotype of SNP rs12979860.

FIGS. 12A and 12B provide a Table showing an estimate of IL28B SNP rs12979860 genotype frequencies for 51 populations for both treatment-naïve and previous therapy failure patients. See, Ge et al., Nature 2009, 461(7262):399-401; Tanaka et al., Nat. Genet. 2009 October; 41(10):1105-9 and Thomas et al., Nature 2009, 461(7265):798-801.

FIG. 13 provides a graph showing patient viral decay data in the context of both the dose of interferon administered the patients in the SCIN-C trial as well as sequence information from the IL28B SNP rs12979860.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. As appropriate, procedures involving the use of commercially available kits and reagents are generally carried out in accordance with manufacturer defined protocols and/or parameters unless otherwise noted.

### DEFINITIONS

By the term “at least 100-700 pg/mL” of interferon-α it is understood that values such as at least 100, 125, 150, 175, 200, 225, 250, 275, 300, 325, 350, 375, 400, 425, 450, 475, 500, 525, 550, 575, 600, 625, 650, 675 or 700 pg/mL can be used to create any specific range of values.

By the term “at least 1 to at least 48 weeks” it is understood that in some instances the therapeutic regimen is administered for a duration of at least 7, 14, 21 or 28 days, while time periods of at least 5, 6, 7, 8, 9, 10, 12, 14, 16, 18, 20, 24, 28, 32, 36, 40, 44, 48, 52, 54, 58, 62, 66, 70, 72 or more weeks can also be selected. In some embodiments of the invention, the therapeutic regimen is administered for a duration of at least 6, 8 or 10 weeks to at least 48 weeks. In other embodiments of the invention, the therapeutic regimen is administered for a duration of at least 6 weeks to at least 32, 36, 40 or 44 weeks. In other embodiments of the invention, the therapeutic regimen is administered for a duration of at least 6 weeks to at least 52, 54, 58, 62, 66, 70, 72 or more weeks.

The term “administer” means to introduce a therapeutic agent into the body of a patient in need thereof to treat a disease or condition.

The term “treating” and/or “treatment” refers to the management and care of a patient having a pathology such as a viral infection or other condition for which administration of one or more therapeutic compounds is indicated for the purpose of combating or alleviating symptoms and complications of those conditions. Treating includes administering one or more formulations of the present invention to prevent the onset of the symptoms or complications, alleviating the symptoms or complications, or eliminating the disease, condition, or disorder. As used herein, “treatment” or “therapy” refer to both therapeutic treatment and prophylactic or preventative measures. In addition, “treating” or “treatment” does not require complete alleviation of signs or symptoms, does not require a cure, and specifically includes protocols which have only a marginal effect on the patient.

The term “therapeutically effective amount” refers to an amount of an agent (e.g. a cytokine such as interferon-α or small molecule inhibitors such as ribavirin) effective to treat at least one sign or symptom of a disease or disorder in a human. Amounts of an agent for administration may vary based upon the desired activity, the diseased state of the patient being treated, the dosage form, method of administration, patient factors such as the patient's sex, genotype, weight and age, the underlying causes of the condition or disease to be treated, the route of administration and bioavailability, the persistence of the administered agent in the body, the formulation, and the potency of the agent. It is recognized that a therapeutically effective amount is provided in a broad range of concentrations. Such range can be determined based on in vitro and/or in vivo assays.

The term “profile” is used according to its art accepted meaning and refers to the collection of results of one or more analyses or examinations of: (1) the presence of; or (2) extent to which an observed phenomenon exhibits various characteristics. Illustrative profiles typically include the results from a series of observations which, in combination, offer information on factors such as, for example, the presence and/or levels and/or characteristics of one or more agents infecting a patient (e.g. the hepatitis C virus), as well as the pharmacokinetic and/or pharmacodynamic characteristics of one or more therapeutic agents administered to a patient as part of a treatment regimen (e.g. interferon-α), as well as the physiological status or functional capacity of one or more organs or organ systems in a patient (e.g. the liver), as well as the genotype of one or more single nucleotide polymorphisms in a patient etc.

The term “therapeutic regimen” is used according to its art accepted meaning and refers to, for example, a part of treatment plan for an individual suffering from a pathological condition (e.g. chronic hepatitis C infection) that specifies factors such as the agent or agents to be administered to the patient, the doses of such agent(s), the schedule and duration of the treatment etc.

The term “pharmacokinetics” is used according to its art accepted meaning and refers to the study of the action of drugs in the body, for example the effect and duration of drug action, the rate at they are absorbed, distributed, metabolized, and eliminated by the body etc. (e.g. the study of a concentration of interferon-α in the serum of the patient that results from its administration via a therapeutic regimen). The term “pharmacodynamics” is used according to its art accepted meaning and refers to the study of the biochemical and physiological effects of drugs on the body or on microorganisms or parasites within or on the body, the mechanisms of drug action and the relationship between drug concentration and effect etc. (e.g. the study of a concentration of hepatitis C virus RNA present in a patient's plasma following one or more therapeutic regimens).

The terms “continuous administration” and “continuous infusion” are used interchangeably herein and mean delivery of an agent such as interferon-α in a manner that, for example, avoids significant fluctuations in the in vivo concentrations of the agent throughout the course of a treatment period. This can be accomplished by constantly or repeatedly injecting substantially identical amounts of interferon-α (typically with a continuous infusion pump device), e.g., at least every hour, 24 hours a day, seven days a week for a period such as at least 1 week to at least 48 weeks, such that a steady state serum level is achieved for the duration of treatment. Continuous interferon-α may be administered according to art accepted methods, for example via subcutaneous or intravenous injection at appropriate intervals, e.g. at least hourly, for an appropriate period of time in an amount which will facilitate or promote in vivo inactivation of hepatitis C virus.

The term “continuous infusion system” refers to a device for continuously administering a fluid to a patient parenterally for an extended period of time or for intermittently administering a fluid to a patient parenterally over an extended period of time without having to establish a new site of administration each time the fluid is administered. The fluid typically contains a therapeutic agent or agents. The device typically has one or more reservoir(s) for storing the fluid(s) before it is infused, a pump, a catheter, cannula, or other tubing for connecting the reservoir to the administration site via the pump, and control elements to regulate the pump. The device may be constructed for implantation, usually subcutaneously. In such a case, the reservoir will usually be adapted for percutaneous refilling.

The term “no detectable HCV-RNA” in the context of the present invention means that there are fewer than 500 and typically fewer than 50 copies of HCV-RNA per milliliter of serum of the patient as measured by quantitative, multi-cycle reverse transcriptase PCR methodology. HCV-RNA is typically measured in the present invention by research-based RT-PCR methodology well known to the skilled clinician. This methodology is referred to herein as HCV-RNA/qPCR. As is known in the art, the lower limit of detection of HCV-RNA can depend upon the specific assay used. For example, art teaches that with the Versant HCV RNA 3.0 Assay [bDNA], the lower limit of quantitation is typically around 615 IU/mL (2.79 log 10 IU/ml); and, with the COBAS AMPLICOR HCV test, v2.0, the lower limit of detection is typically is typically around 50 IU/mL (1.70 log 10 IU/mL). Serum HCV-RNA/qPCR testing and HCV genotype testing can be performed by a central laboratory. See also J. G. McHutchinson et al. (N. Engl. J. Med., 1998, 339:1485-1492), and G. L. Davis et al. (N. Engl. J. Med. 339:1493-1499, the contents of which are incorporated by reference).

The term “patients or humans having hepatitis C infections” as used herein means any patient-including a pediatric patient-having hepatitis C and includes treatment-naive patients having hepatitis C infections and treatment-experienced patients having hepatitis C infections as well as those pediatric, treatment-naïve, and treatment-experienced patients having chronic hepatitis C infections. These patients having chronic hepatitis C include those who are infected with multiple HCV genotypes including type 1 as well as those infected with, for example, HCV genotype 2 and/or 3 and/or 4 etc.

The term “treatment-naive patients having hepatitis C infections” as used herein means patients with hepatitis C who have never been treated with ribavirin and/or any interferon-α, including but not limited to interferon-α, or pegylated interferon-α.

The term “treatment-experienced patients having hepatitis C infections” as used herein means patients with hepatitis C who have been treated with ribavirin and/or any interferon-α, including but not limited to interferon-α, or pegylated interferon-α, including relapsers and non-responders.

The term “patients having chronic hepatitis C infections” as used herein means any patient having chronic hepatitis C and includes “treatment-naive patients” and “treatment-experienced patients” having chronic hepatitis C infections, including but not limited to relapsers and non-responders.

The term “relapsers” as used herein means treatment-experienced patients with hepatitis C who have relapsed after initial response to a conventional course of HCV therapy, e.g. 3-5 MIU pegylated interferon-α administered, for example, in thrice weekly or daily boluses, typically in combination with ribavirin for at least 12 weeks. The term “non-responders” as used herein means treatment-experienced patients with hepatitis C who have not responded to a conventional course of HCV therapy, e.g. e.g. 3-5 MIU pegylated interferon-α administered, for example, in thrice weekly or daily boluses, typically in combination with ribavirin for at least 12 weeks. For descriptions of conventional HCV therapies, see the National Institutes of Health Consensus Development Conference Statement: Management of hepatitis C 2002 (Jun. 10-12, 2002), Gastroenterology 2002; 123(6):2082-2099.

The term “interferon” as used herein means the family of highly homologous species-specific proteins that inhibit viral replication and cellular proliferation and modulate immune response. Human interferons are typically grouped into three classes based on their cellular origin and antigenicity: interferon-α (leukocytes), interferon-β (fibroblasts) and interferon-γ (T cells). Both naturally occurring and recombinant α-interferons may be used in the practice of the invention (e.g. recombinant interferon-α 2a or recombinant interferon-α 2b). Concentrations of interferons such as interferon-α can be quantified a number of ways, for example in picograms per milliliter (e.g. 100 pg/mL) or international units (“IU”, see, e.g. Meager et al., (2001) Establishment of new and replacement World Health Organisation International Biological Standards for human interferon-α and omega. Journal of Immunological Methods, 257, 17-33).

The term “antibody” when used for example in reference to an “antibody capable of binding HCV” is used in the broadest sense and specifically covers intact monoclonal antibodies, polyclonal antibodies, multispecific antibodies (e.g. bispecific antibodies) formed from at least two intact antibodies, and antibody fragments so long as they retain their ability to immunospecifically recognize a target polypeptide.

### ILLUSTRATIVE ASPECTS AND EMBODIMENTS OF THE INVENTION

As noted above, conventional therapeutic regimens designed to eliminate hepatitis C infection fail in about 50% of infected individuals. The instant disclosure provides the results from a clinical trial studying new therapeutic regimens comprising the continuous subcutaneous administration of interferon-α combined with ribavirin in chronic hepatitis C treatment experienced patients. Clinical data obtained from this trial shows that the continuous subcutaneous administration of interferon-α can be used to maintain in vivo concentrations of this therapeutic agent above a critical efficacy threshold for an extended period of time. This clinical data further shows that these therapeutic regimens can eliminate hepatitis C virus in patients previously shown to be refractory to conventional antiviral therapy. Consequently, the therapeutic regimens disclosed herein address a long-felt but unresolved need, specifically the need to eliminate HCV in a greater number of infected individuals than is possible using conventional therapeutic regimens.

Embodiments of the invention involve the continuous subcutaneous administration of interferon-α in order to maintain in vivo concentrations of this therapeutic agent above a critical efficacy threshold in vivo for a sustained period of time. For example, illustrative embodiments of the invention involve the continuous subcutaneous administration of interferon-α in order to maintain in vivo concentrations of this therapeutic agent above at least 100-700 pg/mL (e.g. 300 pg/mL) for at least 1 to at least 48 weeks (a 48-week course of therapy is conventionally recommended for patients infected with HCV genotype 1). In typical embodiments of the invention, the interferon-α concentrations (e.g. 100-700 pg/mL) refer to non-pegylated embodiments of interferon-α 2a or interferon-α 2b (e.g. INTRON®A made by the Schering Corporation). Alternatively, the interferon-α can be pegylated. For embodiments of the invention that comprise pegylated interferon-α, equivalent concentrations can be calculated using art accepted methodologies, for example by calculating the ratio of specific activities and/or molecular weights of: 1) non-pegylated interferon-α such as INTRON®A and 2) pegylated interferon-α such as PegIntron® and then using correlations from such analysis to determine appropriate concentrations of, for example, a pegylated interferon-α.

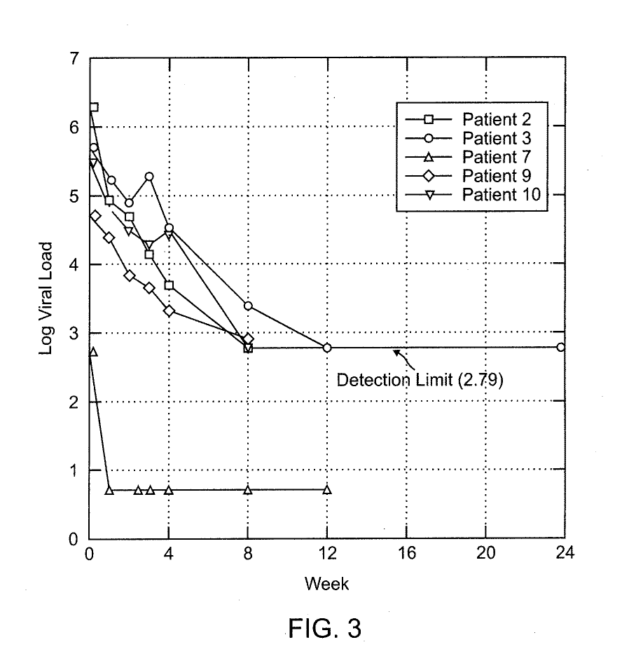

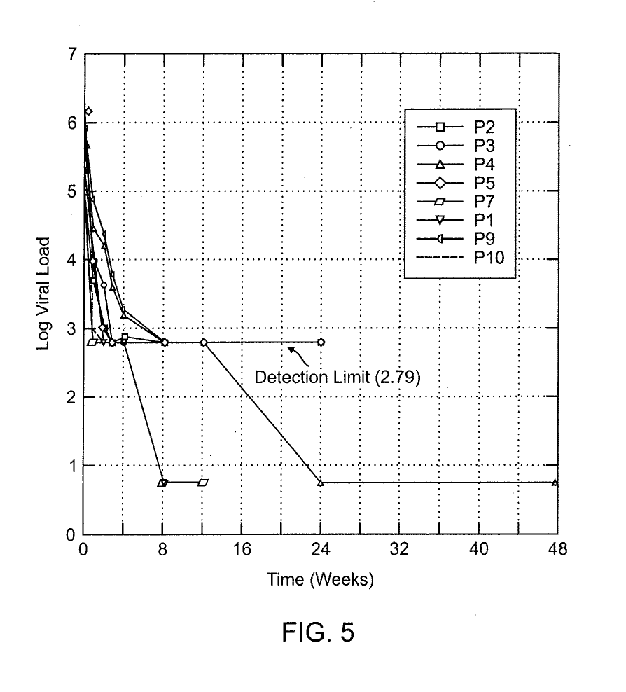

As shown for example by the data disclosed in Example 2 below and shown in associated FIGS. 3-8, delivering concentrations of non-pegylated INTRON-A interferon-α following the therapeutic regimens disclosed herein leads to concentrations of interferon-α that are sustained in vivo and that these sustained in vivo concentrations of interferon-α can be used to eliminate HCV in a greater number of infected individuals than is possible following conventional therapeutic regimens. This is illustrated for example by the robust responses observed in patients enrolled in this trial, patients who had failed to respond to conventional HCV therapy (see, e.g. FIG. 5). Without being bound by a specific scientific theory, the surprising response observed in patients refractory to conventional therapy may result from interferon-α having a efficacy threshold that is: (1) met in only about 50% of patients treated according to conventional therapeutic regimens; and (2) met in a greater number of patients when administered via a continuous infusion apparatus so as to maintain circulating levels of interferon-α in the serum of the patient above a steady state concentration (e.g. at least 100-700 pg/mL) for a sustained period of time (e.g. at least 1 to 48 weeks).

Because the clinical trial disclosed herein comprises patients shown to be refractory to conventional HCV antiviral therapy, the data disclosed in Example 2 below and shown in associated Figures demonstrates the surprising efficacy of therapeutic regimens disclosed herein. In addition, the continuous subcutaneous administration of interferon-α appears to contribute to the reduction of the number and/or the severity of dose dependent side effects observed in patients administered interferon-α according to conventional therapeutic regimens, for example by continuously administering interferon-α in a manner that improves patient tolerance to doses of interferon-α (e.g. as compared to conventional therapeutic regimens that comprise, for example, thrice weekly or daily bolus injections of this cytokine). The ability of these therapeutic regimens to improve the efficacy of interferon-α regimens while simultaneously endeavoring to reduce the dose dependent adverse side effects typically associated with the administration of this cytokine provides an unexpected technical advantage that could not have been predicted based upon what is taught in this technical field.

The invention disclosed herein has a number of embodiments. One illustrative embodiment of the invention is a method of administering interferon-α to a patient infected with hepatitis C virus, the method comprising administering interferon-α to the patient subcutaneously using a continuous infusion apparatus, wherein the interferon-α is administered to the patient using a therapeutic regimen sufficient to maintain circulating levels of the interferon-α in the serum of the patient above a steady state concentration of at least 100 pg/mL for at least 1 to at least 48 weeks. In certain embodiments of the invention, the therapeutic regimen used is sufficient to maintain circulating levels of the interferon-α in the serum of the patient above a steady state concentration of at least 100-700 pg/mL.

In some embodiments of the invention, the therapeutic regimen used is sufficient so that mean circulating levels of the interferon-α in the serum of the patient are above a steady state concentration of at least 100-700 pg/mL for a period of at least 1 to at least 48 weeks. For example, in one such embodiment, the mean circulating levels of the interferon-α in the serum of the patient comprise the average interferon-α serum concentration value of a set of interferon-α serum concentration values measured weekly during the course of therapy (or daily or bimonthly or monthly). In some embodiments of the invention, the therapeutic regimen used is sufficient so that median circulating levels of the interferon-α in the serum of the patient are above a steady state concentration of at least 100-700 pg/mL for a period of at least 1 to at least 48 weeks. For example, in one such embodiment, the median circulating levels of the interferon-α in the serum of the patient comprise the middle interferon-α serum concentration value from a set of interferon-α serum concentration values measured weekly during the course of therapy (or daily or bimonthly or monthly).

As discussed in detail in the sections below, embodiments of the invention include personalized therapeutic regimens tailored to consider one or more characteristics specific to the patient and/or the virus infecting the patient. Embodiments of the invention also include therapeutic regimens tailored to use therapeutic compositions selected to have certain properties (e.g. properties that control the bioavailability profile of a therapeutic agent in the composition). One such embodiment is a method of subcutaneously administering an interferon-α to a patient using a continuous infusion apparatus where the patient is identified as being infected with hepatitis C virus having a specific genotype, for example genotype 1 or genotype 4. In some embodiments of the invention, the patient's prior history of therapy is considered, for example by identifying the patient as a relapser or a non-responder prior to initiating the therapeutic regimen. Embodiments of the invention can further use selected compositions in the therapeutic regimens disclosed herein, for example interferon-α that has undergone a chemical modification process designed to modify one or more bioavailability characteristics, for example conjugation to a polyol (e.g. polyethylene glycol). Alternatively, embodiments of the invention can use interferon-α having a pharmacodynamic and pharmacokinetic profile that more closely mimic interferon-α as found in vivo (e.g. interferon-α not conjugated to a polyol) than the interferon species used in conventional HCV therapies (e.g. Pegasys, Peg-Intron etc.). Without being bound by a specific scientific theory or principle, it is believed that the more natural pharmacodynamic and pharmacokinetic profiles of non-pegylated interferon-α, in combination with continuous and consistent manner in which this polypeptide was administered to patients (e.g. one that reduces or avoids the fluctuations in serum concentrations of interferon-α that occur with thrice weekly or daily administration schedules), contributes to the beneficial outcomes observed in the clinical trial data (see, e.g. Example 2). In certain embodiments, interferon-α is administered to the patient using a therapeutic regimen determined to be sufficient to maintain circulating levels of interferon-α in the serum of the patient above a steady state concentration of at least 100, 125, 150, 175, 200, 225, 250, 275, 300, 325, 350, 375, 400, 425, 450, 475, 500, 525, 550, 575, 600, 625, 650, 675 or 700 pg/mL for at least 1 to at least 48 weeks.

Viral decay data presented in the Figures provides evidence that the therapeutic regimens discussed in the Examples can reduce levels of HCV in patients by at least 1, 1.5, 2, 2.5, 2.7, 3, 3.5, 4, 4.5, 5, 5.5 or 6 orders of magnitude. In this context, in some embodiments of the invention, the therapeutic regimen reduces levels of HCV in the patient by at least 100 to 1,000-fold. In certain embodiments of the invention, the therapeutic regimen reduces levels of HCV in the patient by at least 1,000 to 10,000-fold. In some embodiments of the invention, the therapeutic regimen reduces levels of HCV in the patient by at least 10,000 to 100,000-fold. Typically these methods comprise the concurrent administration of ribavirin (e.g. following a course of administration disclosed in Example 2 below).

One illustrative embodiment of the invention that considers one or more characteristics specific to the patient, for example a patient's unique rate of exogenous interferon-α clearance or metabolism, is a method of administering an interferon-α to a patient infected with hepatitis C virus, the method comprising administering a test dose of interferon-α to the patient and then observing a concentration of circulating interferon-α in the serum of the patient that results from the dose of interferon-α. In such embodiments, the dose of interferon-α (e.g. in a first therapeutic regimen for administering interferon-α) can be administered by any one of a wide variety of methods including bolus injection, multiple intermittent injections, continuous infusion etc. In this embodiment, the concentration of circulating interferon-α that results from the test dose is then used to design a patient-specific therapeutic regimen, one that considers patient specific factors and comprises administering interferon-α to the patient subcutaneously using a continuous infusion apparatus in an amount sufficient to maintain circulating levels of interferon-α in the serum of the patient above a specific in vivo concentration for a specific period of time, for example at least 100 pg/mL for at least 1 to at least 48 weeks. In related embodiments of the invention, the patient-specific therapeutic regimen is selected to: maintain serum interferon-α concentrations in the patient at a value greater than C_{crit}, a concentration threshold that coordinates a patient's sustained response to a therapeutic regimen and/or maintain serum interferon-α concentrations in the patient at a value where the actual efficacy of interferon-α in the patient is greater than the critical efficacy of interferon-α and/or maintain circulating levels of interferon-α in the serum of the patient above a steady state concentration of at least 100, 125, 150, 175, 200, 225, 250, 275, 300, 325, 350, 375, 400, 425, 450, 475, 500, 525, 550, 575, 600, 625, 650, 675 or 700 pg/mL

One specific illustrative embodiment of the invention is a method of administering an interferon-α to a patient infected with hepatitis C virus having genotype 1, 2, 3, 4, 5, or 6, or more preferably genotype 1 or 4, the method comprising administering oral ribavirin to the patient in combination with interferon-α 2a/2b administered subcutaneously using a continuous infusion apparatus, wherein: the patient is identified as a relapser or a non-responder prior to administering the interferon-α; the interferon-α is not conjugated to a polyol; the interferon-α is administered to the patient using a therapeutic regimen sufficient to maintain circulating levels of interferon-α in the serum of the patient above a steady state concentration of at least 100-700 pg/mL for at least 1 to at least 48 weeks; and the therapeutic regimen reduces levels of HCV in the patient by at least 2 logs (100-fold). Certain embodiments of the invention also comprise observing in vitro proliferation of T cells from the patient in response to exposure to interferon-α. For example, as noted in Example 2, the desensitization of the cells for IFN-alfa with regard to T cell proliferation was seen especially in nonresponders at T=24 hrs. Consequently certain embodiments of the invention can use such proliferation assays to obtain information on how a patient may respond to a therapeutic regimen comprising interferon-α. A number of assays of T cell proliferation in response to interferon-α are known in the art that can be adapted for such observations (see, e.g. Folgori et al., Gut, (2006) 55(7): 914-916).

In addition to factors such as HCV genotype and prior treatment history, embodiments of the invention consider additional factors such as a patient's genetic profile and/or physiology (e.g. Body Mass Index). Illustrating this, a number of genetic polymorphisms near the IL28B gene on chromosome 19 are observed to provide information on HCV infected individuals' response to therapeutic regimens comprising interferon-α and ribavirin (see, e.g. Ge et al., Nature 2009, 461(7262):399-401; Tanaka et al., Nat. Genet. 2009 October; 41(10):1105-9; Thomas et al., Nature 2009, 461(7265):798-801; Rauch et al., Gastroenterology 2010 April; 138(4):1338-45; and McCarthy et al., Gastroenterology. 2010 June; 138(7):2307-14, the contents of which are incorporated by reference herein). Illustrative polymorphisms near the IL28B gene are shown in Example 3.

The presence or absence of specific polymorphic variants of the IL28B gene can be used to assess the likelihood of HCV viral clearance following a therapeutic regimen comprising interferon-α and ribavirin as well as to predict the speed of the response to these therapeutic agents. Certain methods of the invention comprise the steps of determining a polynucleotide sequence of a region within 17 kilobases of the IL28B gene on chromosome 19 in the patient (e.g. See the NCBI Single Nucleotide Polymorphisms database (http://www.ncbi.nlm.nih.gov/projects/SNP/) for rs12979860, rs12980275, rs8099917, rs12972991, rs8109886, rs4803223, rs12980602, rs8105790, rs11881222, rs8103142, rs28416813, rs4803219, rs8099917 or rs7248668); and then administering interferon-α to a patient infected with hepatitis C virus by a method comprising administering interferon-α (e.g. interferon-α not conjugated to a polyol) to the patient subcutaneously using a continuous infusion apparatus, wherein the interferon-α is administered to the patient using a therapeutic regimen sufficient to maintain circulating levels of the interferon-α in the serum of the patient above a steady state concentration (e.g. at least 100, 125, 150, 175, 200, 225, 250, 275, 300, 325, 350, 375, 400, 425, 450, 475, 500, 525, 550, 575, 600, 625, 650, 675 or 700 pg/mL). Typically, this therapeutic regimen is sufficient to maintain circulating levels of the interferon-α in the serum of the patient above a steady state concentration for at least 1 week to at least 48 weeks. In certain embodiments of the invention, information on the SNP genotype is used in methods of determining the duration of interferon-α administration (e.g. more than 48 weeks, less than 48 weeks etc.). In certain embodiments of the invention, information on the SNP genotype is used in methods of determining the dose of interferon-α to be administered to the patient. In other embodiments of the invention, information on the SNP genotype is used in methods of determining a target steady state concentration of interferon-α to be maintained in a patient's serum. In one illustrative embodiment of the invention, the SNP is rs12979860 and the method comprises determining if the patient comprises a CC genotype, a TT genotype or a CT genotype. Optionally, the methods are performed on a plurality of patients infected with hepatitis C virus; and the genotype information obtained from the patients is used to stratify patients into different treatment groups (e.g. groups having different IFN dose or regimen duration parameters). Illustrative embodiments of the invention are further discussed in Example 3 below.

A variety of well known methods for the detection of known SNPs are known in the art. Common SNP analysis methods include hybridization-based approaches (see, e.g., J. G. Hacia, Nature Genet., 1999, 21: 42-47), allele-specific polymerase chain reaction (R. K. Saiki et al., Proc. Natl. Acad. Sci. USA, 1989, 86: 6230-6234; W. M. Howell et al., Nature Biotechnol., 1999, 17: 87-88), primer extension (see, e.g., A. C. Syvanen et al., Genomics, 1990, 8: 684-692), oligonucleotide ligation (see, e.g., U. Landegren et al., Science, 1988, 24: 1077-1080) and enzyme-based methods such as restriction fragment length polymorphism and flap endonuclease digestion (see, e.g., V. Lyamichev et al., Nature Biotechnol., 1999, 17: 292-296). One common analysis method includes an initial target amplification step using polymerase chain reaction (PCR) in order to generate a PCR product (see, e.g. R. K. Saiki et al., Science, 1988), followed by an analysis of this product, typically one that includes nucleic acid hybridization to or sequencing of the PCR product. In one such embodiment analysis to determine a person's SNP genotype can be performed for example by real-time polymerase chain reaction (RT-PCR); using Taqman custom designed SNP specific probes (Applied Biosystems) on an ABI HT-7900 instrument using commercially available reagents from Applied Biosystems.

Embodiments of the invention include systems for administering interferon-α to a patient having a hepatitis C infection. In such embodiments of the invention, the system can comprise for example: a continuous infusion pump having a medication reservoir comprising interferon-α; a processor operably connected to the continuous infusion pump and comprising a set of instructions that causes the continuous infusion pump to administer the interferon-α to the patient according to a therapeutic regimen comprising administering interferon-α to the patient subcutaneously; wherein the therapeutic regimen is sufficient to maintain circulating levels of interferon-α in the serum of the patient above a steady state concentration of at least 100-700 pg/mL for at least 1 week to at least 48 weeks.

A related embodiment of the invention is a system for administering interferon-α to a patient having a hepatitis C infection, the system comprising: a continuous infusion pump having a medication reservoir comprising interferon-α; a processor operably connected to the continuous infusion pump and comprising a set of instructions that causes the continuous infusion pump to administer the interferon-α to the patient. In such embodiments of the invention, the system administers interferon-α according to a patient-specific therapeutic regimen made by: administering interferon-α to the patient following a first therapeutic regimen; observing a concentration of circulating interferon-α in the blood of the patient that results from the first therapeutic regimen; and then using the concentration of circulating interferon-α observed to result from the first therapeutic regimen to make a patient-specific therapeutic regimen. Typically in such embodiments, the patient specific therapeutic regimen comprises administering interferon-α to the patient subcutaneously in an amount sufficient to maintain circulating levels of interferon-α in the serum of the patient above a steady state concentration of at least 100-700 pg/mL for at least 1 week to at least 48 weeks.

System embodiments of the invention can be designed for use where the hepatitis C virus is of a specific genotype, for example genotype 1, 2, 3, 4, 5, 6, or more preferably genotype 1 or 4 HCV. In some system embodiments of the invention, the patient is identified as a relapser or a non-responder prior to administering the interferon-α (e.g. interferon-α that is not conjugated to a polyol). Typically in such systems, the therapeutic regimen is sufficient to maintain circulating levels the interferon-α in the patient above a concentration of at least 100, 125, 150, 175, 200, 225, 250, 275, 300, 325, 350, 375, 400, 425, 450, 475, 500, 525, 550, 575, 600, 625, 650, 675 or 700 pg/mL Optionally in such embodiments, the therapeutic regimen is administered for a duration of at least at least 1 week to at least 48 weeks. Typically the therapeutic regimen is sufficient to reduce levels of HCV in the patient by at least 2 logs (100-fold) or 3 logs (1000 fold).

Other embodiments of the invention include interferon-α (e.g. non-pegylated interferon-α 2a or non-pegylated interferon-α) for use in a method of administering interferon-α to a patient infected with hepatitis C virus (HCV), the method comprising administering interferon-α to the patient using a continuous infusion apparatus (e.g. subcutaneously), wherein the interferon-α is administered to the patient using a therapeutic regimen sufficient to maintain circulating levels of the interferon-α in the serum of the patient above a mean steady state concentration of at least 100 pg/mL (or at least 200, 300, 400, 500, 600 or 700 pg/mL) for at least four weeks (or at least 5, 6, 7, 8, 12, 24, 36 or 48 weeks). Typically in such embodiments, the interferon-α is used in a method of administering interferon-α to a patient infected with hepatitis C virus in combination with ribavirin.

It will be apparent to one skilled in the art that various combinations and/or modifications and variations can be made in such therapeutic regimens depending upon the various physiological parameters observed in the patient. For instance, features illustrated or described as part of one embodiment can be used on another embodiment to yield a still further embodiment. For example, the interferon-α for use in a method of administering interferon-α that is noted immediately above, includes the use of this polypeptide in methods that comprise determining a polynucleotide sequence of the patient, wherein the polynucleotide sequence comprises a single nucleotide polymorphism (SNP) designated rs12979860, rs12980275, rs8099917, rs12972991, rs8109886, rs4803223, rs8103142, rs28416813, rs4803219, rs4803217, rs581930, rs8105790, rs11881222, rs7248668 or rs12980602; in particular wherein the SNP is rs12979860 and the method comprises determining if the patient comprises a CC genotype, a TT genotype or a CT genotype. This polynucleotide sequence information can, for example, be used to determine or modulate a parameter of the therapeutic regimen such as a duration of interferon-α administration or an interferon-α dose.

A related embodiment comprises interferon-α for use in a method of administering an interferon-α to a patient infected with hepatitis C virus (HCV), the method comprising administering a test dose of interferon-α to the patient (e.g. following a first therapeutic regimen against HCV); observing a concentration of circulating interferon-α in serum of the patient that results from the test dose of interferon-α; and then using the concentration of circulating interferon-α so observed to make a patient-specific therapeutic regimen, wherein the patient specific therapeutic regimen comprises administering interferon-α to the patient subcutaneously using a continuous infusion apparatus in an amount sufficient to maintain circulating levels of interferon-α in the serum of the patient above a steady state concentration of at least 100 pg/mL. This use can further comprise, for example steps such as: identifying the patient as a relapser or a non-responder; identifying the hepatitis C virus as being a genotype 1 or a genotype 4 virus; observing in vitro proliferation of T cells from the patient in response to exposure to interferon-α; and/or administering interferon-α to the patient using a patient-specific therapeutic regimen sufficient to maintain circulating levels of interferon-α in the serum of the patient above a steady state concentration of at least 200, 300, 400, 500, 600 or 700 pg/mL for at least 4 weeks.

Embodiments of the invention also include a system for administering interferon to a patient having a hepatitis C infection, the system comprising a continuous infusion pump having a medication reservoir comprising interferon-α; a processor operably connected to the continuous infusion pump and comprising a set of instructions that causes the continuous infusion pump to administer the interferon-α to the patient according to a therapeutic regimen comprising administering interferon-α to the patient subcutaneously; wherein the therapeutic regimen is sufficient to maintain circulating levels of interferon-α in the serum of the patient above a steady state concentration of at least 100 pg/mL for at least 4 weeks. Optionally in this system, the processor in the system is used to modulate a parameter of the patient-specific therapeutic regimen using determined polynucleotide sequence information, wherein the parameter comprises a duration of interferon-α administration; or an interferon-α dose.

Yet another embodiment of the invention comprises the use of interferon-α in the manufacture of a composition for treating hepatitis C infection for use in a continuous infusion apparatus, wherein the interferon-α composition is manufactured to allow the continuous infusion apparatus to maintain circulating levels of interferon-α in serum of a patient above a steady state concentration of at least 100 pg/mL for at least 24, 48, 72, 96, 120, 144 or 168 hours (and/or from at least 1 week to at least 48 weeks) when administered subcutaneously. Typically in such embodiments, the interferon-α is not conjugated to a polyol. Optionally in embodiments of the invention, the continuous infusion apparatus is designed for ambulatory use and for example has dimensions smaller than 15×15 centimeters (and typically smaller than 15×15×5 centimeters) and/or is operably coupled to an interface that facilitates the patient's movements while using the continuous infusion pump, wherein the interface comprises a clip, a strap, a snap, a clamp or an adhesive strip.

As noted above, embodiments of the invention are designed to maintain circulating levels of interferon-α in the serum of the patient above a target steady state concentration (e.g. at least 100-700 pg/mL) so as to increase the efficacy of this polypeptide. The term “steady state” is used herein to describe situations in which a variable (e.g. the concentration of circulating interferon-α that results from a therapeutic regimen) remains above a set threshold and/or essentially constant in spite of ongoing processes that strive to change them (e.g. in vivo clearance of exogenous interferon-α by the liver and kidneys). In the context of therapeutic regimens, a steady state is typically reached when the rate of elimination approximates the rate of administration. In this context, the term “above a steady state concentration of at least 100 pg/mL” is used to encompass situations where the patient may exhibit fluctuating interferon-α levels during the course of a therapeutic regimen but these fluctuations in vivo do not drop mean or median circulating levels of interferon-α below a targeted threshold (e.g. at least 100 pg/mL). A related embodiment of the invention is a method of administering an interferon-α to a patient infected with hepatitis C virus, the method comprising administering interferon-α to the patient subcutaneously using a continuous infusion apparatus, wherein the therapeutic regimen is sufficient to maintain circulating levels of interferon-α in the serum of the patient above a target concentration (e.g. 100-700 pg/mL). Such embodiments of the invention can be used to administer interferon-α for a period of at least 1 week to at least 48 weeks.

Some embodiments of the invention include methods for obtaining patient-specific regimen responsiveness profiles based upon individualized patient factors such as infection parameters (e.g. hepatitis C viral load) and therapeutic agent responsiveness parameters (e.g. in vivo concentrations of interferon-α that result from its administration to the patient) and then using the regimen responsiveness profiles to design optimized therapeutic regimens for patients suffering from pathological conditions (e.g. Hepatitis C infections). In particular embodiments, such methods comprise determining patient-specific pharmacokinetic (pK) and pharmacodynamic (pD) parameters (e.g. the concentration of circulating of interferon-α in vivo that results from a specific dose being administered to that patient) and then utilizing these parameters to design new therapeutic regimens. This is typically achieved by adjusting the dose of the therapeutic agent(s) used, or by adjusting the rate or duration of therapeutic agent administration in order to refine a therapeutic regimen and/or optimize the efficacy of a therapeutic regimen. In certain embodiments, the invention provides a computer implemented system for: (1) delivering interferon-α according to an initial dosing parameter (e.g. one disclosed in the Examples below); and/or (2) constructing patient-specific regimen responsiveness profiles based upon a patient's response to the initial dosing parameters; and/or (3) delivering therapeutic agent(s) using optimized therapeutic regimens designed in response to such profiles (e.g. regimens that comprise variations of initial dosing parameters).

In some embodiments of the invention, a patient is administered interferon-α following a set of initial dosing parameters (e.g. those disclosed in the Example below) and the levels of circulating interferon-α in vivo that result from this set of initial dosing parameters are then observed. In this embodiment, the levels of circulating interferon-α in vivo observed in the individual patient are then used to construct one or more further dosing parameters, for example those designed to modulate levels of circulating interferon-α in vivo in that specific patient for some period of time during the course of therapy (e.g. to increase concentrations of circulating interferon-α above a target threshold). Optionally, such embodiments of the invention use therapeutic modelling parameters such as those disclosed in International Application Numbers PCT/US2008/078843 and PCT/US2009/038617, the contents of which are incorporated by reference.

One illustrative embodiment of the invention is a method of using a patient-specific regimen responsiveness profile obtained from a patient infected with hepatitis C virus to design a patient-specific therapeutic regimen such as those disclosed in the Examples below. Embodiments of this method comprise administering at least one therapeutic agent (e.g. interferon-α) to the patient as a test dose (optionally a dose that is part of a first therapeutic regimen) and then obtaining pharmacokinetic or pharmacodynamic parameters from the patient in order to observe a patient-specific response to the test dose. Typically, pharmacokinetic or pharmacodynamic parameters observed comprise a concentration of the therapeutic agent in the blood of the patient that results from the test dose and/or a concentration of hepatitis C virus present in the patient. In this embodiment of the invention, practitioners can then use the pharmacokinetic or pharmacodynamic parameters observed in the patient in response to the test dose (e.g. the concentration of circulating of interferon-α in vivo that results from a specific dose being administered to that patient) to obtain a patient-specific regimen responsiveness profile. This patient-specific regimen responsiveness profile is based upon an HCV infected patient's individualized physiology and necessarily takes into account a variety of host factors such as ethnicity, obesity, insulin resistance, hepatic fibrosis as well as viral factors such as genotype and baseline viral load. This patient-specific regimen responsiveness profile is then used to design a patient-specific therapeutic regimen (e.g. one comprising administering interferon-α to the patient subcutaneously in an amount sufficient to maintain circulating levels of interferon-α in the serum of the patient above a steady state concentration of at least 100-700 pg/mL for at least 1 week to at least 48 weeks).

In typical embodiments of the invention, a therapeutic regimen is selected to control serum interferon-α concentrations in the patient. In illustrative embodiments of the invention, a therapeutic regimen is selected to maintain serum interferon-α concentrations in a patient at a value greater than a critical concentration “C_{crit}” that is associated with therapeutic efficacy, i.e. a concentration threshold that induces and/or facilitates a patient's sustained response to a therapeutic regimen. The term critical concentration “C_{crit},” is used according to its art accepted meaning of: the concentration of a substance (e.g. the concentration of circulating exogenous interferon-α) at and above which functional changes occur in a cell or an organ (see, e.g. Nordberg et al., Pure Appl. Chem., 76, 1033-1082 (2004)). Skilled artisans are familiar with critical efficacy parameters associated with interferon-α in HCV infections (see, e.g. Dahari et al., J Theor Biol 247(2): 371-81 (2007). In certain embodiments of the invention, the critical interferon-α efficacy is the serum concentration of exogenous interferon-α 2b in an individual above which HCV is ultimately cleared, and below which a new chronically infected viral steady-state is reached.

The disclosure provided herein provides further methods for obtaining C_{crit} parameter information. For example, in certain embodiments of the invention, C_{crit} parameter information can be obtained using assessments of a patient or a group of patients' response to one or more predefined therapeutic regimens (e.g. 6 MIU/day, 9 MIU/day and 12 MIU/day as disclosed in Example 2). As discussed in detail below, in other embodiments of the invention, C_{crit} parameter information may be determined empirically and can, for example, consider the pharmacokinetics/pharamacodynamics of the interferon used as well as patient specific factors that can influence this threshold (e.g. the HCV genotype(s) infecting the patient, and/or a patient's weight, treatment history, health status and the like).

In certain embodiments of the invention, the patient-specific therapeutic regimen is designed to maintain plasma interferon-α levels in the patient above a set-point, e.g. above a concentration of at least 100, 125, 150, 175, 200, 225, 250, 275, 300, 325, 350, 375, 400, 425, 450, 475, 500, 525, 550, 575, 600, 625, 650, 675 or 700 pg/mL In other embodiments, the patient-specific therapeutic regimen is selected to modulate interferon-α concentrations in the patient so as to reduce dose-dependent side effects observed during the administration of interferon-α. In some embodiments of the invention, the patient-specific therapeutic regimen is selected to maintain serum interferon-α concentrations in the patient at a value where the actual efficacy of interferon-α in the patient is greater than the critical efficacy of interferon-α. In other embodiments, the patient-specific therapeutic regimen is selected to modulate interferon-α concentrations in the patient so that the patient is administered different interferon-α dosing regimens during different phases of hepatitis C viral load decline.

In certain embodiments of the invention, measurements of phenomena such as the in vivo levels of an administered agent, the actual efficacy and limits of critical efficacy of such agents, as well as the in vivo levels of HCV are determined. Optionally, such determinations are made 0, 1, 2, 3, 4, 6, or 7 days (e.g. week 1) after the administration of a therapeutic regimen and/or any day of weeks 2, 3, 4, 5, 6, 7, 8, 9, 10, 11, 12 etc. up to for example week 48. Certain embodiments of the methods and systems of the invention comprise the administration of interferon-α in a therapeutic regimen that lasts for more than 48 weeks, for example, ones where the therapeutic regimen is administered for 50, 54, 58, 62, 66, 70 or 72 weeks. In one illustrative embodiment, after the initiation of a therapeutic regimen, patients can return for safety and efficacy evaluations on a weekly basis up to week 4 and every 28 days thereafter throughout a 48 week treatment duration, with weekly or monthly follow-up visits up to week 72. Optionally determinations of actual efficacy and limits of critical efficacy occur between 0 and 7 days, and more preferably around between 0 to 2 days. Alternatively, this determination may be made intermittently throughout therapy, to take into account for example individualized patient response to various therapeutic regimens. One with ordinary skill in the art will undoubtedly realize that different pharmacokinetic, pharmacodynamic, and viral kinetic models such as those described herein may be used to achieve this.

In some embodiments of the invention, parameters relating to HCV infection and/or parameters relating to therapeutic regimens for treating HCV infection are examined before the initiation of a therapeutic regimen and/or at one or more times during the administration of a therapeutic regimen and/or after the conclusion of a therapeutic regimen. Such parameters include for example baseline viral load as well as other parameters associated with Hepatitis C infection such as, liver fibrosis or cirrhosis, and/or the presence of serum markers such as alanine transaminase (ALT). Such parameters further include biochemical markers that are induced in response to interferon-α (e.g. interferon-α administered according to a therapeutic regimen) such as neopterin and 2′,5′-oligoadenylate synthetase (OAS).

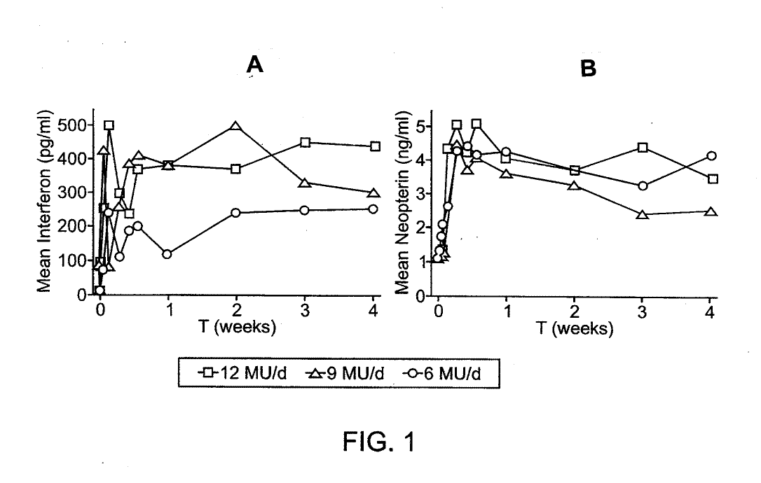

Exemplary embodiments of the invention that comprise the observation of one or more parameters relating to HCV infection and/or parameters relating to therapeutic regimens for treating HCV infection include methods and/or systems for administering interferon-α to a patient infected with hepatitis C virus that are sufficient to increase levels of neopterin by at least 10, 20, 30, 40 or 50% as compared to pretreatment levels. In some embodiments of the invention, the therapeutic regimen is sufficient to increase levels of neopterin by at least 1, 2, 3, or 4 ng/mL (see, e.g. FIG. 1). In other embodiments, a method and/or system for administering interferon-α to a patient infected with hepatitis C virus uses a therapeutic regimen sufficient to increase levels of 2′,5′ oligo-adenylate synthetase by at least 2, 4, 6, 8 or 10-fold as compared to pretreatment levels. In some embodiments of the invention, the therapeutic regimen is sufficient to increase levels of 2′,5′ oligo-adenylate synthetase by at least 25, 50, 75 or 100 pg/dL. Methods and materials used in the measurement of neopterin are described for example in Fernandez et al., J Clin Gastroenterol. 2000 30(2):181-6). Commercially available neopterin tests include those offered by Quest laboratories Teterboro, New Jersey, test number 97402P and HENNING test, BRAHMS Diagnostica GmbH, D-12064, Berlin, Germany. Methods and materials used in the measurement of 2′,5′ oligo-adenylate synthetase are described for example in Podevin et al., J. Hepatol. 1997 (2):265-71). Methods and materials used in the measurement of beta-2-microglobulin are described for example in Malaquarnera Eur J Gastroenterol Hepatol. 2000 August; 12(8):937-9.

Another commonly examined secondary indicator of effectiveness of a treatment regimen is a change in the levels of serum alanine aminotransferase (ALT). In general, an ALT level of less than about 80, less than about 60, less than about 50, or about 40 international units per liter of serum is considered normal. In this context, in some embodiments of the invention, a therapeutic regimen disclosed herein reduces ALT levels to less than about 200 IU/L, less than about 150 IU/L, less than about 125 IU/L, less than about 100 IU/L, less than about 90 IU/L, less than about 80 IU/L, less than about 60 IU/L, or less than about 40 IU/L. Certain embodiments of the invention comprises a method and/or system for administering interferon-α to a patient infected with hepatitis C virus sufficient to decrease levels of alanine transaminase (ALT) by at least 2, 3, 4 or 5-fold as compared to pretreatment levels. In some embodiments of the invention, the therapeutic regimen is sufficient to decrease levels of alanine transaminase by at least 25, 50, 75 or 100 IU/L. Methods and materials used in the measurement of alanine transaminase (and/or aspartate transaminase) are described for example in Sterling et al., Dig Dis Sci. 2008 May; 53(5):1375-82 Epub 2007.

As noted above, embodiments of the invention can examine for example, levels of neopterin and/or 2′,5′ oligo-adenylate synthetase and/or ALT in a patient as well as the other markers disclosed herein and/or known in the art to, for example, examine the pretreatment status of a patient and/or assess the course of a therapeutic regimen and/or design patient specific therapeutic regimens. Embodiments of the invention can also examine a combination of these parameters and/or additional parameters such as a level of beta-2-microglobulin in plasma of the patient; a genotype or quasispecies of the hepatitis C virus; a patient's prior medical treatment history; and/or a presence or degree of a side effect that results from the first therapeutic regimen and/or the presence of serum markers associated with liver fibrosis. Serum markers of liver fibrosis further include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

As illustrated by the disclosure in the Examples below, in embodiments of the invention one can also observe a presence or degree of factors such as a depression, a neutropenia, a thrombocytopenia, as well as one or more systemic flu-like symptoms that can result from the administration of interferon-α. Methods and materials used in the measurement of depression are well known in the art (e.g. the Beck Depression Inventory) and are described for example in Golub et al., J Urban Health. 2004 June; 81(2):278-90). Methods and materials used in the measurement of neutropenia and thrombocytopenia are well known in the art and described for example in Koskinas et al., Med. Virol. 2009 Mar. 24; 81(5):848-852 and Nudo et al., Can J Gastroenterol. 2006 September; 20(9):589-92.

As noted above, embodiments of the invention provide technical advantages in this art by eliminating HCV in a greater number of infected individuals than possible using conventional therapeutic regimens. Other technical advantages of embodiments of the invention include, for example, the reduction or elimination of detrimental side effects that can result from the interferon-α administered according to conventional therapeutic regimens. For example, in typical embodiments of the invention, the continuous infusion of interferon-α allows this cytokine to reach high circulating concentrations in vivo while concurrently reducing or eliminating the adverse immunological and/or hematological reactions that can occur for example when this cytokine is administered in a bolus (e.g. a bolus of interferon-α that is administered 3 times a week etc.). In this context, embodiments of the invention include the administration of a dose of interferon-α to a patient using a continuous infusion apparatus in order to reduce or eliminate the incidence of neutropenia, and/or thrombocytopenia and/or the induction of autoimmune diseases that are observed when this cytokine is administered in a bolus (e.g. conventional HCV therapies). Exemplary embodiments of the invention include the administration of a dose of interferon-α to a patient using a continuous infusion apparatus so as to reduce or eliminate the incidence of adverse immunological and/or hematological reactions such as neutropenia, and/or thrombocytopenia and/or the induction of autoimmune diseases (e.g. thyroiditis) by at least 10, 20, 30, 40 or 50% as compared to therapeutic regimens where this cytokine is administered in a bolus.

A wide variety of therapeutic regimens can be designed using the methods and/or systems disclosed herein. In typical embodiments of the invention, the therapeutic regimen comprises administering interferon-α using a continuous infusion pump wherein the regimen is sufficient to maintain circulating levels of interferon-α in the serum of the patient above a steady state concentration of at least 100-700 pg/mL for at least 1 to at least 48 weeks. Typically the therapeutic regimen comprises the administration of an additional anti-viral agent such as ribavirin, VX-950, SCH 503034, R1626, or R71278. The administration of such agents can be modulated over the course of a therapeutic regimen. For example, in certain embodiment of the invention, the patient-specific therapeutic regimen comprises administering a first dose of interferon-α (and/or ribavirin) for a first time period and/or phase of hepatitis C viral decline and a second dose of interferon-α (and/or ribavirin) for a second time period and/or a second phase of hepatitis C viral decline.

Once a therapeutic regimen (e.g. one disclosed in Example 1 or 2 below) is selected and administered, practitioners can then obtain a patient-specific regimen responsiveness profile that results from the administration of this therapeutic regimen. The patient-specific regimen responsiveness profiles can then be used to design further patient-specific therapeutic regimens. For example, certain embodiments of the invention comprise obtaining pharmacokinetic or pharmacodynamic parameters from the patient so as to observe a patient-specific response to a first therapeutic regimen as discussed above, wherein the pharmacokinetic or pharmacodynamic parameters comprise at least one of: a concentration of administered interferon-α in the plasma of the patient; or a concentration of hepatitis C virus in the plasma of the patient; using the pharmacokinetic or pharmacodynamic parameters observed in the patient in response to the first patient-specific therapeutic regimen to obtain a second patient-specific regimen responsiveness profile; and using the second patient-specific regimen responsiveness profile to design a second (or third or fourth etc.) patient-specific therapeutic regimen. A number of aspects of such personalized therapeutic regimens are discussed in Example 3 below.

As discussed in detail below, embodiments of the invention are performed using computer systems. Typically, the computer is operatively coupled to an infusion pump that delivers interferon-α to a patient according to instructions provided by the computer. Optionally, the systems include a controller programmed with mathematical models representing a viral response in a patient receiving a therapeutic regimen and programmed to regulate the dosing rate of therapeutic agent based on the models and the measurements of clinical parameters (e.g. in vivo concentrations of an administered therapeutic agent or viral load). In certain embodiments of the invention, the controller program is use to modulate the dose of interferon-α administered to the patient, the interferon-α administration profile, the duration of interferon-α administration or the like.

One such embodiment of the invention is a method of administering interferon-α to a patient suffering from a Hepatitis C infection, the method comprising: administering interferon-α to the patient following a first therapeutic regimen; obtaining pharmacokinetic or pharmacodynamic parameters from the patient to observe a patient-specific response to the first therapeutic regimen wherein the parameters comprise a concentration of interferon-α in the blood of the patient that results from the first therapeutic regimen; or a concentration of hepatitis C virus present in the patient. The pharmacokinetic or pharmacodynamic parameters so observed in the patient in response to the first therapeutic regimen are then used to design a patient-specific therapeutic regimen; one which can, for example, be programmed into a controller that operably coupled to a continuous infusion pump. The continuous infusion pump having this program can then be used to administer interferon-α to the patient according to the controller programming, programming that, for example, controls one or more aspects of an administration profile (e.g. the timing of the administration, the rate of administration etc.).

As discussed in detail below, embodiments of the invention include systems such as those that comprise computer processors and the like coupled to a medication infusion pump and adapted to deliver interferon-α according to a specific therapeutic regimen. Typically, these systems comprise one or more control mechanisms designed to modulate delivery of interferon-α, for example those that allow its delivery according to a predetermined infusion profile. For example, in some embodiments of the invention, a processor is programmed to control a therapeutic regimen that includes an infusion profile designed to take into account one or more characteristics of the patient (e.g. weight) and/or one or more characteristics of the hepatitis virus infecting the patient (e.g. genotype) and/or one or more characteristics of the therapeutic agent administered to the patient (e.g. the presence or absence of a polyethylene glycol moiety). Optionally such profiles are selected from a plurality of predetermined infusion profiles that are stored in the computer system.

In one illustrative embodiment of the invention, a system comprising one or more computer processors is coupled to a medication infusion pump in order to administer a therapeutic regimen designed in accordance with the total interferon-α per kilogram and/or total interferon-α per day that is administered to the patient. In a related embodiment of the invention, a system administers a therapeutic regimen designed to consider the weight and/or body-mass index (BMI) of the patient (e.g. to increase or, alternatively, decrease the dose or duration of interferon-α administered in accordance with a patient's current weight). For example, a therapeutic regimen designed to consider the weight of the patient can consider selecting a weight-based dose of continuously administered interferon-α (e.g. INTRON A) of 80 kIU/kg/day, or alternatively 120 kIU/kg/day, or alternatively 160 kIU/kg/day.

In another embodiment of the invention, a system administers a therapeutic regimen designed to consider the past and/or current viral load observed in the patient (e.g. to increase or, alternatively, decrease the dose or duration of interferon-α administered in accordance with the patient's current viral load). In another embodiment of the invention, a system administers a therapeutic regimen designed to consider the specific genotype of the hepatitis virus that infects the patient (e.g. to increase or, alternatively, decrease the dose or duration of interferon-α administered in accordance with the patient's HCV genotype). In another embodiment of the invention, a system administers a therapeutic regimen designed to consider the presence and/or past or current levels of serum markers such as alanine transaminase, neopterin, 2′,5′-oligoadenylate synthetase and the like in the patient (e.g. to increase or, alternatively, decrease the dose or duration of interferon-α administered in accordance with the patient's past and/or current levels of serum markers). In some embodiments, the therapeutic regimen may be based on a single factor, e.g., the patient's weight only. In other embodiments, therapeutic regimen is based upon multiple factors.

In another embodiment of the invention, a polynucleotide sequence of the patient using the system is determined, the polynucleotide sequence comprising a single nucleotide polymorphism (SNP) designated rs12979860, rs12980275, rs8099917, rs12972991, rs8109886, rs4803223, rs8103142, rs28416813, rs4803219, rs4803217, rs581930, rs8105790, rs11881222, rs7248668 or rs12980602; and the processor in the system is used to modulate a parameter of the patient-specific therapeutic regimen using determined polynucleotide sequence information, for example, one where the parameter comprises a duration of interferon-α administration or an interferon-α dose.

In some computer implemented embodiments of the invention, the controller is programmed so that the continuous infusion pump administers interferon-α in a manner that: maintains serum interferon-α concentrations in the patient at a value greater than C_{crit}, a concentration threshold that coordinates a patient's sustained response to a therapeutic regimen; maintains serum interferon-α concentrations in the patient at a value where the actual efficacy of interferon-α in the patient is greater than the critical efficacy of interferon-α; modulates interferon-α concentrations in the patient so that the patient is administered different interferon-α dosing regimens during different phases of hepatitis C viral load decline; modulates interferon-α concentrations in the patient so that a difference between the actual efficacy of interferon-α and the critical efficacy of interferon-α in the patient is increased; or modulates interferon-α concentrations in the patient so as to reduce adverse side effects observed during the administration of interferon-α. In another computer implemented embodiment of the invention, the controller is operatively coupled to the continuous infusion pump and programmed so that the pump administers interferon-α to a patient infected with HCV according to a therapeutic regimen in a manner that: maintains serum interferon-α concentrations in the patient at a value less than a EC₅₀, a concentration at which the effectiveness of interferon-α is 50% of its maximum. In an illustrative computer implemented embodiment of the invention, the controller is operatively coupled to the continuous infusion pump and programmed so that the pump administers interferon-α at a dose and for a period of time (e.g. at least 1 to at least 48 weeks) selected to maintain a plasma interferon-α concentration above a set-point (e.g. 100-700 pg/mL) for the period of time; and the therapeutic regimen further comprises administering a nucleoside analog that interferes with Hepatitis C viral replication (e.g. ribavirin).

In certain embodiments of the invention, the system for administering interferon-αis coupled to an electronic system for managing medical data on an electronic communication network. For example one such electronic system can comprise at least one electronic server connectable for communication on the communication network, the at least one electronic server being configured for: receiving a first physiological parameter observed in a patient (e.g. a patient's viral load or a patient's serum concentration of interferon-α) setting a test dose of the interferon-α for infusion by the continuous infusion pump (e.g. one designed to test how quickly the exogenous interferon-α is cleared by a patient's liver and kidneys) based on the first physiological parameter; receiving second physiological parameter information of the patient indicative of a response of the patient to the interferon-α of the test dose; and then setting a second dose of the interferon-α for infusion by the continuous infusion pump, based on the second physiological parameter. Illustrative electronic systems for managing medical data on an electronic communication networks that can be adapted for use with embodiments of the invention are described, for example, in U.S. Publication No. 20090246171, the contents of which are incorporated by reference.

Yet another embodiment of the invention is a program code storage device, comprising: a computer-readable medium; a computer-readable program code, stored on the computer-readable medium, the computer-readable program code having instructions, which when executed cause a controller operably coupled to a medication infusion pump to administer the interferon-α to a patient infected with the hepatitis C virus according to a patient-specific therapeutic regimen made by: administering interferon-α to the patient following a first therapeutic regimen obtaining pharmacokinetic or pharmacodynamic parameters from the patient so as to observe a patient-specific response to the first therapeutic regimen wherein the pharmacokinetic or pharmacodynamic parameters comprise at least one of: a concentration of interferon-α in the blood of the patient that results from the first therapeutic regimen; or a concentration of hepatitis C virus present in the patient; using the pharmacokinetic or pharmacodynamic parameters observed in the patient in response to the first therapeutic regimen to obtain a patient-specific regimen responsiveness profile; and then using the patient-specific regimen responsiveness profile to make the patient-specific therapeutic regimen.

The methods of the invention can be practiced on a wide variety of individuals infected with HCV including those previously treated for HCV infection or having a specific HCV strain. For example, some embodiments of the invention include the step of selecting the patient for treatment by identifying them as one previously treated with a course of interferon-α therapy, wherein the previous course interferon-α therapy was observed to be ineffective to treat one or more symptoms associated with the HCV infection (e.g. was a non-responder or a relapser). Other embodiments of the invention include the step of selecting the patient for treatment by identifying the patient as one infected with a specific HCV genotype, for example one infected with Genotype 1, 2, 3, 4, 5 or 6.

In certain embodiments of the invention, the status of HCV in the individual is monitored during one or more of the phases of the viral life cycle. In particular, during chronic HCV infection, the level of serum HCV RNA does not vary significantly (<0.5 log) on time scales of weeks to months. However, when patients chronically infected with HCV are treated with interferon-α (IFN) or IFN plus ribavirin, HCV RNA generally declines after a 7-10 hour delay. The typical decline is biphasic and consists of a rapid first phase lasting for approximately 1-2 days during which HCV RNA, on average, may fall 1 to 2 logs in genotype 1 infected patients and as much as 3 to 4 logs in genotype 2 infected patients. Subsequently, a slower second phase of HCV RNA decline ensues. Triphasic viral declines also have been observed in some patients. A triphasic decline consists of a first phase (1-2 days) with rapid virus load decline followed by a shoulder phase (4-28 days)—in which virus load decays slowly or remains constant—and a third phase of renewed viral decay. In nonresponders, there may be no viral decline (null response) or a first phase followed by no second-phase decline (flat partial response) or rebound to baseline level.

In certain embodiments of the invention, the status of HCV in the individual is monitored during one or more of the phases of the viral life cycle so as to obtain information useful in the tailoring of the therapeutic regimen to the viral phase in a specific individual. Typically, in certain embodiments of the invention, the initial and then changing concentrations of hepatitis C virus in the serum of the patient can be measured by a quantitative PCR method that is employed during the various phases of the viral decline that occurs in response to one or more therapeutic regimens. In one illustrative embodiment, the status of HCV in the individual is monitored over a period of time so as to determine if one or more therapeutic regimens is sufficient to reduce the levels of hepatitis C virus at least 1, 2, 3, 4, 5 or 6 logs. In another illustrative embodiment, the status of HCV in the individual is monitored over a period of time so as to determine if a therapeutic regimen is sufficient to reduce the concentration of hepatitis C virus to below the detection limit of the assay (typically 10-100 IU/mL of serum or plasma; e.g. during the first, second or third phases and/or at the junctions between these different phases of hepatitis C viral decline). In the embodiments of the invention that examine viral load, those of skill in the art understand that units of viral load, which are expressed a number of ways in the literature including: (1) IU/mL—international units/mL; (2) (RNA) copies/mL; and (3) virions/mL (see, e.g. Saldanha et al., Vox Sang 1999; 76:149-158).

In some embodiments, interferon-α may be administered at a first dosing rate during the first stage and a second dosing rate during the second stage, higher than the first dosing rate, i.e. or resulting in higher efficacy than the first dosing rate, followed by a dosing rate calculated to result in efficacy determined by fitting the viral model. By way of non-limiting example, the first stage may last between at least 1 and 12 weeks, more preferably between at least 3 to 5 weeks, and more preferably for at least 4 weeks. The second stage may last for at least 2 to 4 weeks. Finally, for the remainder of the therapy, the patient may be administered interferon-α at a dosing rate adjusted based on patient's actual and critical efficacy as described above. In one specific embodiment, the first dosing rate may be set to about 3 to 9 MIU/day (based on a 75 kg patient), preferably about 6 MIU/day, and the dosing rate during the second stage may be set to about 9 MIU/day to about 20 MIU/day, preferably to about 12 MIU/day/75-kg patient. Alternatively, interferon-α may be administered at a dosing rate calculated to result in higher efficacy or maximized difference between actual efficacy and critical efficacy first. The first stage may then be followed by a stage with lower efficacy, by a stage where efficacy is calculated as described above, or both.

Interferons for use in embodiments of the invention include interferon α-2b (Intron A) (which is not pegylated) and pegylated interferon α-2b (PegIntron, PEG-IFN). Embodiments of the invention can include doses of Intron A that rage from at least 3, 6, 9, 12 million or more IU/day. Continuous SC delivery of Intron A can be achieved via the Medtronic MiniMed Paradigm infusion system for 24, 26, 48, 60, 72 etc. weeks of therapy. Typically, patients will also receive 1000-1600 mg/day oral ribavirin by mouth daily based upon weight (e.g. 1000 mg/day if weight ≦75 kg; 1200 mg/day if weight >75 kg etc.). Individuals in such studies can include those with HCV genotype 1 infection who have had no previous interferon-α treatment, or alternatively HCV genotype 1 or 4 infection non-responders (e.g. individuals who have had previous interferon-α treatment but relapsed etc.).

In embodiments of the invention, a patient's response to various therapeutic regimens administered according to embodiments of the invention can be examined by a variety of methods known in the art. Typical efficacy variables can be assessed in response to an HCV infected patient's treatment regimen and can include for example assessments of rapid virologic response (RVR): Undetectable HCV RNA level in response to a certain therapeutic regimen; as well as early virologic response (EVR): ≦2-log₁₀ reduction in HCV RNA level in response to a certain therapeutic regimen as compared with the baseline level etc.

Further illustrative methods and materials useful in practicing embodiments of the invention are discussed in detail below.

### Illustrative Methods and Materials For Observing HCV in Embodiments of the Invention

Hepatitis C virus is a positively stranded RNA virus that exists in at least six genetically distinct genotypes. These genotypes are designated Type 1, 2, 3, 4, 5 and 6, and their full length genomes have been reported (see, e.g. Genbank/EMBL accession numbers Type 1a: M62321, AF009606, AF011753, Type 1b: AF054250, D13558, L38318, U45476, D85516; Type 2b: D10988; Type 2c: D50409; Type 3a: AF046866; Type 3b: D49374; Type 4: WC-G6, WC-G11, WG29 (Li-Zhe Xu et al, J. Gen. Virol. 1994, 75: 2393-98), EG-21, EG-29, EG-33 (Simmonds et al, J. Gen. Virol. 1994, 74: 661-668), the contents of which are incorporated by reference). In addition, viruses in each genotype exist as differing “quasispecies” that exhibit minor genetic differences. The vast majority of infected individuals are infected with genotype 1, 2 or 3 HCV. HCV infection affects approximately 1.8% of the population in the USA and 3% of the population of the world. In over 85% of infected people, HCV causes a lifelong infection characterized by chronic hepatitis that varies in severity between individuals.

A person suffering from chronic hepatitis C infection may exhibit one or more of the following signs or symptoms which can be examined (typically in addition to other factors) in order to obtain a patient-specific profile: (a) elevated serum alanine aminotransferase (ALT), (b) positive test for anti-HCV antibodies, (c) presence of HCV as demonstrated by a positive test for HCV-RNA, (d) clinical stigmata of chronic liver disease, (e) hepatocellular damage. Such criteria may not only be used to diagnose hepatitis C, but can be used to evaluate a patient's response to drug treatment. Elevated serum ALT and aspartate aminotransferase (AST) are known to occur in uncontrolled hepatitis C, and a complete response to treatment is generally defined as the normalization of these serum enzymes, particularly ALT (Davis et al., 1989, New Eng. J. Med. 321:1501-1506). ALT is an enzyme released when liver cells are destroyed and is symptomatic of HCV infection. Interferon-α causes synthesis of the enzyme 2′,5′-oligoadenylate synthetase (2′5′OAS), which in turn, results in the degradation of the viral mRNA. Houglum, 1983, Clinical Pharmacology 2:20-28. Increases in serum levels of the 2′5′OAS coincide with decrease in ALT levels. Histological examination of liver biopsy samples may be used as a second criteria for evaluation. See, e.g., Knodell et al., 1981, Hepatology 1:431-435, whose Histological Activity Index (portal inflammation, piecemeal or bridging necrosis, lobular injury and fibrosis) provides a scoring method for disease activity, the contents of which are incorporated by reference.

As discussed in detail below, certain embodiments of the invention include the step of monitoring the HCV viral load in a subject and to adjust the therapeutic regimen based upon the observed result. Similarly, in certain embodiments of the invention, whether a particular method or methodological step (e.g. a specific regimen) is effective in combating an HCV infection can be determined by a number of factors, typically by measuring viral load. Alternatively, in certain circumstances, one can measure another parameter associated with HCV infection, including, but not limited to, liver fibrosis.

Viral load can be measured by a variety of procedures known in the art, for example, by measuring the titer or level of virus in serum. These methods include, but are not limited to, a quantitative polymerase chain reaction (PCR) and/or a branched DNA (bDNA) test. Many such assays are available commercially, including a quantitative reverse transcription PCR (RT-PCR) (Amplicor HCV Monitor™ Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Illustrative assays used in embodiments of the invention to monitor viral titer in the methods of the invention include the COBAS Hepatitis C Virus (HCV) TaqMan Analyte-Specific Reagent Assay and/or the COBAS Amplicor HCV Monitor V2.0 and/or the Versant HCV bDNA 3.0 Assays (see, e.g. Konnick et al., Journal of Clinical Microbiology, May 2005, p. 2133-2140, Vol. 43, No. 5, the contents of which are incorporated by reference).

In certain embodiments of the invention, and HCV infected individual is administered a therapeutic agent such as interferon-α and/or a small molecule inhibitor such as ribavirin and the response to such agents is then observed by monitoring changes in the levels of HCV-RNA that are detectable in vivo, for example HCV-RNA copy number per milliliter of blood. In this context, an appropriate therapeutic response is associated with decreasing levels of HCV-RNA that are detectable in the blood of an infected individual. Ideally, a therapeutic regimen will reduce this number so that there is no longer any detectable HCV-RNA.

While viral titers are the most important indicators of effectiveness of a dosing regimen, other parameters can also be measured as secondary indications of effectiveness. Secondary parameters include reduction of liver fibrosis; and reduction in serum levels of particular proteins. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20, the contents of which are incorporated by reference. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems. Another alternative but indirect method of determining viral load is by measuring the level of serum antibody to HCV. Methods of measuring serum antibody to HCV are standard in the art and include enzyme immunoassays, and recombinant immunoblot assays, both of which involve detection of antibody to HCV by contacting a serum sample with one or more HCV antigens, and detecting any antibody binding to the HCV antigens using an enzyme labeled secondary antibody (e.g., goat anti-human IgG). See, e.g., Weiss et al. (1995) Mayo Clin. Proc. 70:296-297; and Gretch (1997) Hepatology 26:43 S-47S, the contents of which are incorporated by reference.

### Illustrative Therapeutic Agents for Use in Embodiments of the Invention

Embodiments of the invention can use a wide variety of therapeutic agents known in the art to both construct patient-specific profiles and then deliver therapeutic agent(s) using optimized regimens based upon these profiles. Typical embodiments of the methods disclosed herein include the administration of interferon-α (also termed “interferon-alpha”) to an individual infected with HCV. Such embodiments of the invention optimize regimens for treating HCV infection using permutations of ribavirin and an interferon-α treatments that are well known in the art, e.g., as disclosed in U.S. Pat. No. 6,299,872, U.S. Pat. No. 6,387,365, U.S. Pat. No. 6,172,046, U.S. Pat. No. 6,472,373, and U.S. Patent Application No. 200060257365. The term “interferon-alpha (interferon-α)” as used herein means the highly homologous cytokine polypeptides that inhibit viral replication and cellular proliferation and modulate immune response. As is known in the art, interferon-α includes human interferon-α 2a and 2b (collectively designated herein “interferon-α 2a/2b”), almost identical interferon-αpolypeptides that bind to the same specific cell surface receptor complex known as the IFN-α receptor (IFNAR) and which differ by only a single basic amino acid (lysine versus arginine). Due to their extreme similarity, medical practitioners can use either interferon-α 2a or interferon-α 2b in combination with ribavirin to treat HCV infection. In this context, skilled artisans teach, for example, that comparisons of HCV therapeutic regimens that use either interferon-α 2a or interferon-α 2b in combination with ribavirin show that there are no significant differences in the efficacy and safety of these two almost identical polypeptides (see, e.g. Laguno et al., Hepatology 2009 49(1): 22-31; Scott et al., Drugs 2008 68(6): 791-801; Yenice et al., Turk J Gastroenterol 2006 17(2): 94-98; and Kim et al., Korean J Hepatol 2008 14(4): 493-502, the contents of which are incorporated by reference).

Interferon-alphas include, but are not limited to, recombinant interferon alfa-2b such as Intron-A interferon available from Schering Corporation, Kenilworth, N.J., recombinant interferon alfa-2a such as Roferon interferon available from Hoffmann-La Roche, Nutley, N.J., recombinant interferon-α2c such as Berofor alpha 2 interferon available from Boehringer Ingelheim Pharmaceutical, Inc., Ridgefield, Conn., interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain, or a consensus alpha interferon such as those described in U.S. Pat. Nos. 4,897,471 and 4,695,623 and the specific product available from Amgen, Inc., Newbury Park, Calif., or interferon alfa-n3 a mixture of natural alpha interferons made by Interferon Sciences and available from the Purdue Frederick Co., Norwalk, Conn., under the Alferon Tradename or recombinant interferon alpha available from Frauenhoffer Institute, Germany or that is available from Green Cross, South Korea. The use of interferon alfa-2a or alpha 2b to treat HCV is typical. Since interferon alpha 2b, among all interferons, has the broadest approval throughout the world for treating chronic hepatitis C infection, it is most typical. Methods for the manufacture of interferons are described for example in U.S. Pat. Nos. 4,530,901 and 5,741,485.

Various interferons available on the market include, but are not limited to alpha interferons ((IFN-α): Roferon®-A, Intron®-A; consensus IFN: Infergen®, and the like)); and beta interferons ((IFN-βs): Betaseron®, Rebif®, Avonex®, Cinnovex® and Berlex)). Pegylated interferon-alpha-2b was approved in January 2001 and pegylated interferon-alpha-2a was approved in October 2002. Examples of commercially available pegylated interferons include, but are not limited to, PEGASYS®, PegIntron™ and Reiferon Retard®. As is known in the art, different preparations of therapeutic molecules such as the interferons do not exhibit identical activities and such activities are therefore published by the manufacturer. For example, for Infergen, the published activity is 1×10⁹ U/mg or 1 MIU/ug. For Pegylated Interferon alpha 2b, (PegIntron) the published (package insert) is 0.7×10⁸ U/mg or 70,000 U/ug. For Pegylated interferon alpha 2a (Pegasys) the published data suggest that the pegylated product has 7% the activity of the non-pegylated product. In typical embodiments, bio-potent non-pegylated interferon-alpha (IFN-α-2a or IFN-α-2b) or consensus interferon is used.

A number of alpha interferons are approved for use for the treatment of hepatitis. Intron-α (interferon-α 2b, Schering Plough) was a first interferon-α approved for hepatitis C use. Intron-A is also indicated for a variety of cancer therapies including a list of hematological malignancies and hepatitis B. There is no mention of therapy failures in the Intron-α package insert, however the label for Intron-α plus ribavirin therapy is indicated only for naïve patients. Roferon (interferon-α 2a, Roche) is another interferon-α approved for hepatitis C. There is no indication of use with ribavirin and no discussion of therapy failures in the package labeling. Infergen (interferon-α consensus, Valeant) is labeled only for hepatitis C. Peg-Intron (interferon-α 2b pegylated with a 12 kD PEG (polyethylene glycol), Schering Plough) was the first pegylated interferon-α introduced to the marketplace. Pegylation of the interferon-α leads to a molecule with reduced biological activity but a greatly increased circulating half-life in-vivo. Peg-Intron is labeled for weight based dosing with a single weekly injection in combination with ribavirin. Peg-intron is only labeled for naïve patients. The half-life of Peg-Intron is about 48 hours, so plasma levels of interferon-α are essentially zero by the end of day 7 following bolus injection. Pegasys (interferon-α 2a pegylated with a 40 kD PEG, Roche) was the second pegylated interferon-α approved for clinical use. In contrast to Peg-Intron, Pegasys is typically delivered at the same dose for all patients; however the ribavirin component is typically dosed by weight Like Peg-Intron, Pegasys is only indicated for interferon-α naïve patients. The pharmacokinetics of Pegasys are considerably different than Peg-intron due to the larger molecular weight of the PEG attached to the interferon-α. The circulating half-life of Pegasys is about 3 weeks, which might have considerable safety implications in the case of overdosing but does not allow for significantly reduced trough levels in the plasma.

As noted above, certain embodiments of the methods disclosed herein include the administration of interferon-α that is conjugated to a polyol such as polyethylene glycol. Such interferon-α conjugates can be prepared by coupling an interferon alpha to a variety of water-soluble polymers. A non-limiting list of such polymers include polyethylene and polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described in U.S. Pat. No. 4,766,106, U.S. Pat. No. 4,917,888, European Patent Application No. 0 236 987, European Patent Application Nos. 0510 356, 0 593 868 and 0 809 996 (pegylated interferon alfa-2a) and International Publication No. WO 95/13090. The typical polyethylene-glycol-interferon alfa-2b conjugate is PEG₁₂₀₀₀-interferon alpha 2b. The phrases “12,000 molecular weight polyethylene glycol conjugated interferon alpha” and “PEG₁₂₀₀₀-IFN alpha” as used herein mean conjugates such as are prepared according to the methods of International Application No. WO 95/13090 and containing urethane linkages between the interferon alfa-2a or -2b amino groups and polyethylene glycol having an average molecular weight of 12000.

In certain embodiments of the invention, an interferon-α administered in one or more sequential phases of a therapeutic regimen is not conjugated to a polyol. In some embodiments of the invention, the interferon-α so administered comprises two interferon-α species: a first interferon-α species that is conjugated to a polyol; and a second interferon-α species that is not conjugated to a polyol. Optionally different species of interferon-α are administered in one or more of the different sequential phases of the invention.

To minimize the number of pump refills during the therapy, the supply of interferon-α in the pump may last for an extended period of time. Because the loadable amount of interferon-α is fixed by the drug reservoir volume, to increase the amount of time the interferon-α supply may last, potency of interferon, as well as concentration of interferon-α may be increased. Accordingly, in some embodiments, the interferon-α may comprise a highly potent interferon. The term “highly potent” means an interferon-α that may exhibit favorable characteristics such as antiviral activity, antiproliferative activity, efficacy in clearing hepatitis virus from cells, increased ratio of antiviral activity to antiproliferative activity, or increased ratio of Tₕ1 differentiation activity to antiproliferative activity. Due to these characteristics, less volume of interferon-α is required to cause the same therapeutic effect on the patient, and thus highly potent interferon-α formulation may be administered at a lower flow rate. Alternatively, a highly soluble interferon-α may be used to prepare formulations with increased concentration of interferon, which can also be administered at a lower flow rate. The term “highly soluble” means interferon-α with a solubility of between at least 5 mg/mL to at least 10 mg/mL In typical embodiments, the interferon-α concentration may be at least 10 MIU/mL, 20 MIU/mL, 30 MIU/mL, 40 MIU/mL, 50 MIU/mL, 60 MIU/mL, 70 MIU/mL, 80 MIU/mL, 90 MIU/mL, 100 MIU/mL, 125 MIU/mL, 150 MIU/mL, 175 MIU/mL, 200 MIU/mL and 225 MIU/mL to at least 1500 MIU/mL Typically the interferon-α concentration is at least 25 MIU/mL

In practicing the methods of the invention, the therapeutic regimen(s), e.g. the therapeutic agent(s), the dosage amount(s), dosage period(s), dosage schedule(s), dosage route(s), and so on, for agents such as interferon-α and/or ribavirin, encompass those generally used in the art to administer these agents in a manner that typically produces an improvement in one or more physiological conditions associated with a chronic hepatitis C infection. In this context, skilled artisans understand that a variety of therapeutic regimens known in the art can be employed in and/or adapted to the methods of the invention (e.g. those described in United States Patent Applications 2006/0088502 and 2006/0024271 and U.S. Pat. No. 6,849,254).

Medical personnel can control and/or modify an interferon-α dosage regimen depending on the constellation of clinical factors observed in a specific individual (factors which are known to change during treatment). In particular, artisans understand that for HCV infections, one single predetermined regimen is not applicable to all patients and that optimally effective regimens are typically those that are individually designed in view of various factors observed in a specific individual. For example, medical personnel may select a specific interferon-α dosage regimen based upon the genotype or subtype of HCV that is observed to be infecting the patient and/or the amount of HCV-RNA per ml of serum in the patient as measured by a quantitative PCR method. As is similarly known in the art, the dosage regimen may be selected or controlled depending on the weight and age of a patient, whether the patient is known to be a nonresponder or relapser, or whether the patient is observed to have another pertinent pathological condition (e.g. cirrhosis of the liver, hepatocarcinoma, HIV infection, or the like). Depending upon, for example, the constellation clinical factors observed in a specific individual and/or the personal needs of these patients, interferon-α can be administered via a variety of routes, for example subcutaneously, intramuscularly or intravenously.

In certain HCV therapeutic regimens described in the art, an infusion delivery device (e.g. a medication infusion pump) has been used to deliver interferon-α. These studies include those described in Carreno et al. J Med Virol 1992; 37:215-219; Schenker et al., Journal Interferon Cytokine Res. 1997; 17:665-670; and Tong et al., Hepatology. 2003; 38 (No. 4 Supplement 1):81A. However, even many years after these clinical studies of therapeutic regimens that included the continuous infusion of interferon-α, no data has been reported regarding the elucidation of treatment relevant physiological mechanisms associated with such methods, much less how to use such methods to address the long felt needs in this area of technology (i.e. the need to eliminate HCV in a greater number of infected individuals than is possible using conventional therapeutic regimens). Significantly, these prior studies did not focus on the serum levels of interferon-α achieved in their protocols, much less any associations of these levels with efficacies of treatment.

The following descriptions of various illustrative schemes for administering therapeutically effective amounts of the combination therapy of interferon-α and ribavirin are not limiting and are instead provided merely as typical examples of dosage regimens known in the art that can be employed and/or adapted to the methods of the invention. In typical embodiments of the invention, the interferon-α administered is selected from one or more of interferon alpha-2a, interferon alpha-2b, a consensus interferon, a purified interferon alpha product (e.g. a purified interferon-α product produced by a recombinant technology) and/or a pegylated interferon-α. As is known in the art, an interferon-α dose can be characterized in international units (IU) or milligrams of polypeptide, optionally in the context of amount of agent per kilogram of patient weight and/or another measure of patient size (e.g. m²). Optionally, the interferon-α can be selected from consensus interferon, interferon alpha-2a, interferon alpha-2b, or a purified interferon-α product and the amount of interferon-α administered can be from at least 1 to at least 20 million IU per day via continuous infusion.

In certain embodiments of the invention, interferon-α can be administered in different doses during different phases of the viral cycle that are observed in HCV therapy. For example, in one such embodiment of the invention, different doses of interferon-α are administered during the first and/or second phases of viral decline and/or shoulder and/or final phase of viral decline and can include for example a first dose between 6-20 MIU (e.g. at least 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 MIU) daily for a first specific time period (e.g. 2 weeks), followed by a second different dose between 6-20 MIU daily for another time period (e.g. 6 weeks), followed by a third dose between 6-20 MIU daily for yet another time period (e.g. 16 weeks to 24 weeks). Such dosage regimes can use an infusion delivery device (e.g. a medication infusion pump) programmed to deliver different doses of interferon-α during different stages of a treatment regimen.

Since interferon-α may be exposed to elevated temperatures and/or mechanical stresses for an extended period of time, it may be desirable to prepare interferon-α compositions that enhance the stability of the interferon-α and prevent its degradation. In one embodiment, interferon-α may be stabilized in an aqueous medium by a mixed buffer system. For example, U.S. Pat. No. 6,734,162 discloses methods and materials that may be employed to prepare such compositions. Various other methods known and used in the art may also be used.

Because interferons may cause adverse side effects, in some embodiments, they may be delivered in a manner that provides increased levels of the drug in liver tissues and decreased levels in non-liver tissues. In one embodiment, it may be accomplished by chemically modifying the interferon-α to render it inactive until the modification is cleaved off by a liver-specific enzyme. One example of such technology, known as HepDirect, is offered by Metabasis Therapeutics, Inc, La Jolla, Calif. In another embodiment, the interferons may be modified to enhance its site-specific delivery to target cells. Suitable compounds for modifying the interferons in this manner include, but are not limited to, lactosaminated albumin, (Stefano, J. Pharmacol. Exp. Ther., May 2002; 301: 638-642) or galactosylated poly(L-lysine) (Gal-PLL) (Zhu et al., Bioconjugate Chem., 19 (1), 290-298, 2008). In yet other embodiments, interferon-α may be delivered via a drug delivery device either intraperitoneally or directly to the liver, slightly upstream from the liver vascular bed, such as into the hepatic artery.

In vivo samples (e.g. blood, serum, plasma, tissue etc.) may be assayed for interferon-α concentrations using a variety of different methods known and used in the art. One suitable example is an electrochemiluminescence-based assay and an ORIGEN analyzer (IGEN International, Inc. Gaithersburg, Md.) as disclosed for example in Obenauer-Kutner et al., Journal of Immunological Methods, Volume 206, Issues 1-2, 7 Aug. 1997, Pages 25-33. Other methods used in the art include those disclosed for example in Niewold et al., Genes Immun. 2007; 8:492-502; Pirisi et al., Digestive Diseases and Sciences, 42(4): 767-7771 (1997); Christeff et al., European Journal of Clinical Investigation. 32(1):43-50, January 2002; Sibbitt et al., Arthritis & Rheumatism, Volume 28 Issue 6, Pages 624-629, 2005; and Lam et al., Digestive Diseases and Sciences, 42(1):178-85 (1997). In addition, ELISA kits designed to provide quantitative assays of interferon-α concentrations in serum (e.g. 100-700 pg/mL) are commercially available from vendors, including for example the Human IFN-alpha Platinum ELISA CE available from Bender MedSystems® (e.g. Product # BMS216CE) and The Human IFN alpha colorimetric ELISA Kit (Serum Samples) available from Thermo Scientific Life Science Research Products (e.g. Product # 411101). Those of skill in the art understand that, information on the specific activity of an interferon-α (e.g. 2.6×10⁸ IU/mg for INTRON® A), readily allows one to characterize an interferon-α in terms of both picograms and IU.

In some embodiments of the invention, interferon-α may be administered to a patient in combination with other antiviral agent(s). Combination therapy is particularly desirable for patients who suffer from an ongoing (chronic) hepatitis infection. Suitable anti-viral agents include, for example HCV polymerase or protease inhibitors. These anti-viral agents are typically administered orally.

Embodiments of the methods disclosed herein include the administration of ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The in vitro inhibitory concentrations of ribavirin are disclosed in Goodman & Gilman's “The Pharmacological Basis of Therapeutics”, Ninth Edition, (1996) McGraw Hill, New York, at pages 1214-1215. The Virazole product information discloses a dose of 20 mg/mL of Virazole aerosol for 18 hours exposure in the 1999 Physicians Desk Reference at pages 1382-1384. Typical ribavirin dosage and dosage regimens are also disclosed by Sidwell, R. W., et al. Pharmacol. Ther 1979 Vol 6. pp 123-146 in section 2.2 pp 126-130. Fernandes, H., et al., Eur. J. Epidemiol., 1986, Vol 2(1) pp1-14 at pages 4-9 disclose dosage and dosage regimens for oral, parenteral and aerosol administration of ribavirin in various preclinical and clinical studies.

Suitable examples of ribavarin include, but are not limited to, Copegus®, Rebetol® Ribasphere®, Vilona®, Virazole®, in addition to generic versions of the drug. Ribavirin is typically available in 200-mg capsules with the daily dosage calculated based on patient's weight or viral genotype. A person with ordinary skill in the art will undoubtedly be capable of determining the proper dosage of ribavirin to be administered. For example, for patient with viral genotype 1, the daily dosage may be 1,200 mg for patients that weigh over 165 lbs and 1,000 mg for patients that weigh less than 165 lbs. On the other hand, for patients with viral genotypes 2 or 3, the daily dosage may be set to 800 mg regardless of the patient's weight. Suitable inhibitors include, but are not limited to, telapravir and others described below and in U.S. Pat. Nos. 5,371,017, 5,597,691, and 6,841,566.

Ribavirin is typically administered as part of a combination therapy to a patient in association with interferon-α, that is, before, after or concurrently with the administration of the interferon-α. The interferon-α dose is typically administered during the same period of time that the patient receives doses of ribavirin. The amount of ribavirin administered concurrently with the interferon-α typically varies depending upon various factors such as a patient's weight and can be less than 399 mg per day or from 400 to 1600 mg per day, e.g. 600 to 1200 mg/day, or 800 to 1200 mg day, or 1000 to 1200 mg a day, or 1200 to 1600 mg a day. In certain embodiments of the invention, the amount of ribavirin administered to a patient concurrently with pegylated interferon-α can be for example from at least 8 to at least 15 mg per kilogram per day, typically at least 8, 12 or 15 mg per kilogram per day, in divided doses.

Those of skill in the art understand that embodiments of the invention include administering interferon-α and ribavirin either alone or in combination in methods for obtaining patient-specific regimen responsiveness profiles and then using the regimen responsiveness profiles to design optimal therapeutic regimens for patients suffering from pathological conditions such as Hepatitis C infections. In addition, there are a number of other HCV therapeutic agents known in the art in addition to interferon-α and ribavirin that can be administered either alone or in combination with interferon-α and/or ribavirin in order to obtain patient-specific regimen responsiveness profiles and then using the regimen responsiveness profiles to design optimal therapeutic regimens for patients suffering from pathological conditions such as Hepatitis C infections. Such anti-viral agents include for example, but are not limited to, immunomodulatory agents, such as thymosin; VX-950, CYP inhibitors, amantadine, and telbivudine; Medivir's TMC435350, GSK 625433, R1626, ITMN 191, other inhibitors of hepatitis C proteases (NS2-NS3 inhibitors and NS3/NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (see, e.g., compounds of U.S. Pat. Nos. 5,807,876, 6,498,178, 6,344,465, 6,054,472, WO 97/40028, WO 98/40381, WO 00/56331 the contents of which are incorporated by reference, and mycophenolic acid and derivatives thereof, and including, but not limited to VX-497, VX-148, and/or VX-944); or combinations of any of the above. A variety of such inhibitors which may be used in these methods are known in the art and described below (see, e.g. Sheldon et al., Expert Opin Investig Drugs. 2007 August; 16(8):1171-81).

In some embodiments of the invention, a therapeutic agent used in combination with interferon-α is VX-950. VX-950 (also termed (Telaprevir) is an orally active targeted antiviral therapy for hepatitis C virus infection that has been shown to reduce plasma HCV RNA in patients with genotype 1 virus (see, e.g. U.S. Patent Nos. 20070218138 and 20060089385, the contents of which are incorporated by reference). In some embodiments, the dose of amorphous VX-950 can be a standard dose, e.g., at least 1 g to at least 5 g a day, more typically at least 2 g to at least 4 g a day, more typically at least 2 g to at least 3 g a day, e.g., at least 2.25 g or at least 2.5 g a day. For example, a dose of at least 2.25 g/day of amorphous VX-950 can be administered to a patient, e.g., at least 750 mg administered three times a day. Such a dose can be administered, e.g., as three 250 mg doses three times a day or as two 375 mg doses three times a day. In some embodiments, the 250 mg dose is in an 700 mg tablet. In some embodiments, the 375 mg dose is in an 800 mg tablet. As another example, a dose of 2.5 g/day of amorphous VX-950 can be administered to a patient, e.g., 1250 mg administered two times a day. As another example, at least 1 g to at least 2 g of amorphous VX-950 a day can be administered to a patient, e.g., at least 1.35 g of amorphous VX-950 can be administered to a patient, e.g., at least 450 mg administered three times a day. Vertex Pharmaceuticals Incorporated has disclosed results from an ongoing Phase 2b study evaluating Telaprevir-based treatment in patients with genotype 1 chronic hepatitis C virus infection who did not achieve sustained virologic response (SVR) with at least one prior pegylated interferon (peg-IFN-α) and ribavirin (RBV) regimen. In this study, 52% (60 of 115; intent-to-treat analysis) of patients randomized to receive treatment with a 24-week Telaprevir-based regimen (12 weeks of Telaprevir in combination with peg-IFN-α and RBV, followed by 12 weeks of peg-IFN and RBV alone) maintained undetectable HCV RNA 12 weeks post-treatment (SVR12).

In some embodiments of the invention, a therapeutic agent used in combination with interferon-α is SCH 503034. SCH 503034 is another hepatitis C virus protease inhibitor (see, e.g. U.S. Patent Nos. 20070224167, 20060281688, 20070185083, 20070099825, and Sarazzin et al., Gastroenterology. 2007 April; 132(4):1270-8. Epub 2007, the contents of which are incorporated by reference). Illustrative dosing regimens for SCH 503034 include 200 mg, 300 mg, or 400 mg, 3 times daily orally. For example, genotype-1 patients in a 14-day course of treatment (5 treatment arms including 1 placebo arm), showed an HCV RNA reduction with the maximum HCV reduction of more than 2 logs in the group receiving 400 mg of SCH503034. SCH503034 was safe and well-tolerated with no serious adverse events. Schering-Plough Corporation disclosed results from an analysis of a Phase II trial of Boceprevir which showed a high rate of sustained virologic response (SVR) in patients receiving Boceprevir-based combination therapy in a study of 595 treatment-naïve patients with chronic hepatitis C virus genotype 1. In a 48-week treatment regimen, the SVR rate at 12 weeks after the end of treatment (SVR 12) was 74 percent (ITT) in patients who received 4 weeks of PEGINTRON (peginterferon alfa-2b) and REBETOL® (ribavirin, USP) prior to the addition of Boceprevir (800 mg TID) (P/R lead-in), compared to 38 percent for patients in the control group receiving 48-weeks of PEGIntron And REBETOL alone. Patients in the study who received 48-weeks of Boceprevir in combination with PEGIntron and REBETOL from the beginning of treatment, (no PegIntron/ribavirin (P/R) lead-in) achieved 66 percent SVR 12. In the two 28-week Boceprevir arms of the study, SVR at 24 weeks after the end of treatment (SVR 24) was 56 percent and 55 percent for patients in the lead-in and no lead-in arms, respectively. Importantly, for patients who received the PEGIntron And REBETOL lead in and had rapid virologic response (RVR), defined as undetectable virus (HCV-RNA) in plasma after 4 weeks of Boceprevir treatment, SVR (ITT) was 82 percent in the 28-week regimen and 92 percent in the 48 week regimen. See also, Njoroge et al. Acc Chem. Res. 2008 January; 41(1):50-9.

In some embodiments of the invention, a therapeutic agent used in combination with interferon-α is Medivir's TMC435350 (see, e.g. the disclosure presented at the 14th International Symposium on Hepatitis C Virus and Related Viruses in Glasgow, Scotland by Simmen et al. entitled “Preclinical Characterization of TMC435350, a novel macrocyclic inhibitor of the HCV NS3/4A serine protease”, the contents of which are incorporated by reference). This disclosure demonstrates the ability of TMC435350 to reduce the amount of Hepatitis C virus replication in laboratory replicon experiments via protease inhibition. In addition, this disclosure notes that combinations of TMC435350 with interferon-α is also reported to enhance RNA reduction (>4 logs reduction in the replicon model), and to suppress the appearance of drug-resistance. Results presented at 43rd annual meeting of the European Association for the Study of the Liver show that TMC435350 was well tolerated during 5 days of dosing, and provoked a strong and rapid antiviral activity in genotype 1 infected individuals. See, e.g. Reesink et al., Safety of the HCV protease inhibitor TMC435350 in healthy volunteers and safety and activity in chronic hepatitis C infected individuals: a phase I study, 43rd annual meeting of the European Association for the Study of the Liver (EASL 2008), Milan, 2008.

In some embodiments of the invention, a therapeutic agent used in combination with interferon-α is ITMN 191 (see, e.g. U.S. Patent Application No. 20050267018, the contents of which are incorporated by reference). InterMune reports that dosing in a Phase 1a single ascending-dose (SAD) trial of ITMN-191 in healthy subjects shows no serious adverse events were reported in the SAD trial. Preliminary safety data from the SAD trial provide evidence that ITMN-191 was well tolerated and safe at the doses intended for the Phase 1b multiple-ascending dose of ITMN-191. InterMune additionally reported that, based on a preliminary review of the available and still blinded clinical data from the four completed cohorts of the Phase 1b study, ITMN-191 was safe and well-tolerated.

In some embodiments of the invention, a therapeutic agent used in combination with interferon-α is GSK 625433. A study presented at the 42nd annual meeting of the European Association for the Study of the Liver (EASL 2007) disclosed GSK625433 as a highly potent and selective inhibitor of genotype 1 HCV polymerases that is observed to be synergistic with interferon-in vitro.

In some embodiments of the invention, a therapeutic agent used in combination with interferon-α is Taribavirin. Taribavirin (formerly known as viramidine) is an oral pro-drug of ribavirin that is less likely to cause anemia. In a study presented at the 43rd annual meeting of the European Association for the Study of the Liver (EASL 2008) in Milan, investigators disclosed results from an open-label Phase IIb trial, 278 treatment-naive patients with genotype 1 chronic hepatitis C stratified by body weight and baseline viral load and randomly assigned (1:1:1:1) to receive taribavirin at doses of 20, 25, or 30 mg/kg/day, or else weight-based ribavirin (800, 1000, 1200, or 1400 mg/day), all administered with pegylated interferon alfa-2b (PegIntron). Baseline patient characteristics were generally similar across the study arms with regard to factors predictive of treatment response.

In some embodiments of the invention, a therapeutic agent used in combination with interferon-α is a nucleoside having anti-HCV properties, such as those disclosed in WO 02/51425 (4 Jul. 2002), assigned to Mitsubishi Pharma Corp.; WO 01/79246, WO 02/32920, WO 02/48165 (20 Jun. 2002), and WO2005/003147 (13 Jan. 2005) (including R1656, (2′R)-2′-deoxy-2′-fluoro-2′-C-methylcytidine, methylcytidine, shown as compounds 3-6 on page 77) assigned to Pharmasset, Ltd.; WO 01/68663 (20 Sep. 2001), assigned to ICN Pharmaceuticals; WO 99/43691 (2 Sep. 1999); WO 02/18404 (7 Mar. 2002), US2005/0038240 (Feb. 17, 2005) and WO2006021341 (2 Mar. 2006), including 4′-azido nucleosides such as R1626, 4′-azidocytidine, assigned to Hoffmann-LaRoche; U.S. 2002/0019363 (14 Feb. 2002); WO 02/100415 (19 Dec. 2002); WO 03/026589 (3 Apr. 2003); WO 03/026675 (3 Apr. 2003); WO 03/093290 (13 Nov. 2003);: US 2003/0236216 (25 Dec. 2003); US 2004/0006007 (8 Jan. 2004); WO 04/011478 (5 Feb. 2004); WO 04/013300 (12 Feb. 2004); US 2004/0063658 (1 Apr. 2004); and WO 04/028481 (8 Apr. 2004); the content of each of which is incorporated herein by reference in its entirety. For example, patients given oral doses of R1626, (500 mg, 1500 mg, 3000 mg, 4500 mg) achieved viral load reductions of 1.2, 2.6, and 3.7 log 10 in the 100 mg, 300 mg and 4500 mg doses respectively. R1626 was generally well-tolerated with increasing adverse events at the highest dose (4500 mg). No viral resistance was found. Investigators disclosed data on R1626 at the 43rd annual meeting of the European Association for the Study of the Liver (EASL) showing that R1626 produces good response with pegylated interferon/ribavirin and has high barrier to resistance. See, e.g. Nelson et al., High End-of-Treatment Response (84%) After 4 Weeks of R1626, Peginterferon Alfa-2a (40 kd) and Ribavirin Followed By a Further 44 Weeks of Peginterferon Alfa-2a and Ribavirin. 43rd annual meeting of the European Association for the Study of the Liver (EASL 2008), Milan 2008; and Pogam et al., Low Level of Resistance, Low Viral Fitness and Absence of Resistance Mutations in Baseline Quasispecies May Contribute to High Barrier to R1626Resistance In Vivo. 43rd annual meeting of the European Association for the Study of the Liver (EASL 2008), Milan, 2008.

In some embodiments of the invention, a therapeutic agent used in combination with interferon-α is R71278, a polymerase inhibitor developed by Roche and Pharmasset. With R71278, there is a dose-dependent antiviral activity across all dosing arms with the 1,500 mg twice-daily arm achieving a great than 99% decrease in HCV RNA (viral load). R7128 is reported to be generally safe and well-tolerated with no serious adverse events or any dose reductions due to adverse events. Pharmasset, Inc. has disclosed results of a clinical trial evaluating R7128 1000 mg twice daily (BID) in combination with the standard of care (SOC), Pegasys plus ribavirin, in 31 treatment-naive patients chronically infected with hepatitis C virus genotype 1. See, e.g. Lalezari et al., Inhibitor R7128 with Peg-IFN-α and Ribavirin: Interim Results of R7128 500 mg BID for 28 Days. 43rd annual meeting of the European Association for the Study of the Liver (EASL 2008), Milan, 2008.

Methods for formulating the interferon, ribavirin and other therapeutic agent compositions of the invention for pharmaceutical administration are known to those of skill in the art. See, for example, Remington: The Science and Practice of Pharmacy, 19^{th} Edition, Gennaro (ed.) 1995, Mack Publishing Company, Easton, Pa. Typically the therapeutic agents used in the methods of the invention combined with at pharmaceutically acceptable carrier. The term “pharmaceutically acceptable carrier” is used according to its art accepted meaning and is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration.

Therapeutic compositions of cytokines such as interferon-α and compounds such as ribavirin can be prepared by mixing the desired cytokine having the appropriate degree of purity with optional pharmaceutically acceptable carriers, excipients, or stabilizers in the form of lyophilized formulations, aqueous solutions or aqueous suspensions (see, e.g. Remington: The Science and Practice of Pharmacy Lippincott Williams & Wilkins; 21 edition (2005), and Ansel's Pharmaceutical Dosage Forms and Drug Delivery Systems Lippincott Williams & Wilkins; 8th edition (2004)). For example, pharmaceutical compositions of pegylated interferon alpha-suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g. NaCl), preservatives (e.g. thimerosol, cresol or benzylalcohol), and surfactants (e.g. tween or polysorabates) in sterile water for injection. Acceptable carriers, excipients, or stabilizers are typically nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Solutions or suspensions used for administering a cytokine can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose.

Suitable carriers for formulations of interferons in liquid form include, but are not limited to, water, saline solution, buffered solutions, blood, glucose, concentrated plasma, concentrated or fractioned blood, glycerol or any combination thereof. Acceptable excipients or stabilizers that may be added to interferon-α formulations are nontoxic to recipients at the dosages and concentrations employed, and include buffers and preservatives typically used in the art. The formulations herein may also comprise other active molecules as necessary for the particular indication being treated. A person with ordinary skill in the art is capable of selecting active molecules with complementary activities that do not adversely affect each other in amounts that are effective for the purpose intended. In different embodiments, the formulation may also include bioactive agents including, neurotransmitter and receptor modulators, anti-inflammatory agents, anti-viral agents, anti-tumor agents, antioxidants, anti-apoptotic agents, nootropic and growth agents, blood flow modulators and any combinations thereof.

In addition, interferon-α may be incorporated into a sustained release composition designed to continuously administer interferon-α over a period of time. The interferons may, for example, be entrapped in a microsphere prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in, for example, Remington's Pharmaceutical Sciences, Lippincott Williams & Wilkins; 21 edition (May 1, 2005). Alternatively, the interferons may be incorporated into semipermeable matrices of biodegradable solid polymers. The matrices may be in the form of shaped articles, e.g., films, rods, or pellets. Suitable materials for sustained-release matrices include, but are not limited to, poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof. Polymers such as ethylene-vinyl acetate and lactic acid-glycolic acid enable release of molecules for over 100 days. Processes for preparing sustained-release compositions are well known and are described, for example, in U.S. Pat. No. 6,479,065.

### Determining Patient-Specific Pharmacokinetic and Pharmacodynamic Parameters:

In certain embodiments of the invention, one or more algorithms is used to obtain a regimen responsiveness profile that can be used for example to design and/or modify a therapeutic regimen administered to a patient (see, e.g. International Application Number PCT/US2009/038617, the contents of which are incorporated by reference). Typically, an algorithm is used to determine patient-specific parameters such as the in vivo concentrations of therapeutic agent(s) administered to a patient, the baseline viral load, liver fibrosis or cirrhosis, or presence (e.g. in the serum of the patient) of markers associate with a pathological condition such as alanine transaminase (ALT) or aspartate transaminase (AST). The algorithm(s) can further be used to design an optimized therapeutic regimen (e.g. an interferon-α dose that is, for example, calculated to avoid severe side effects that can be associated with interferon-α therapy). In embodiments of the invention, the patient may then be tested a plurality of times for the interferon-α serum concentration or the viral load or any other relevant parameters known to those of ordinary skill in the art. A plurality of patient-specific pharmacokinetic and pharmacodynamic parameters may be obtained by fitting the pharmacokinetic and pharmacodynamic models known in the art (and described herein) to this data. In addition, a wide variety of statistical techniques known and used in the art, such as for example, linear or non-linear regressions, may be employed in embodiments of the invention. In some embodiments, the models or their solutions in analytical or numerical form may be combined or substituted into each other as is commonly done by artisans skilled in this technology.

In certain embodiments of the invention, a first therapeutic regimen can include a dose interferon-α given to the patient in order to obtain information on the rate at which the patient metabolizes the interferon-α (e.g. to ascertain the dose of interferon-α in that patient that is required to produce a median concentration in serum of at least 100-700 pg/mL. In other embodiments of the invention, a first therapeutic regimen can include a dose of an interferon-α and ribavirin that is therapeutically effective yet calculated to avoid substantial adverse side effects, and can be determined by one with ordinary skill in the art from experience, population data, journal articles, etc. By way of non-limiting example, regular interferon-α can be administered at a dosing rate at, or approximately at, a rate of 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 million or more international units (MIU) per day via a continuous infusion apparatus. Optionally, the levels of circulating interferon-α that result from this first therapeutic regimen can then be observed and, if necessary, the regimen can then be modified to, for example, maintain circulating levels of interferon-α in that patient above a target threshold, for example 100-700 pg/mL. Those of skill in the art can readily adapt existing protocols associated with various interferons (e.g. non-pegylated interferon-α 2a, non-pegylated interferon-α 2b and the like) to use with continuous infusion apparatus.

HCV therapeutic regimens of the invention typically comprise administering multiple therapeutic agents. For example, in addition to interferon-α, patients can also receive a dose of an antiviral compound such as 1000-1600 mg/day oral ribavirin by mouth daily based upon weight (e.g. 1000 mg/day if weight ≦75 kg; 1200 mg/day if weight >75 kg etc.). Of course, a person with ordinary skill in the art will undoubtedly appreciate that these specific doses for interferon-α and ribavirin are provided only as a benchmark, and such person will be capable of customizing them depending on patient specific factors. Such factors may include, but are not limited to, patient's response to therapy, patient's ability to tolerate high dosage of interferon, viral genotype, viral kinetics, whether the patient was a prior non-responder or a treatment-naïve, extent of virus, and so forth.

In certain embodiments of the invention, it can be advantageous to vary the dose of one or more therapeutic agents in order to obtain better estimates of the pK and pD parameters as well as to determine whether these parameters have changed. In some of these embodiments, interferon-α may be administered by more than one method, i.e., bolus injection and continuous infusion. In other embodiments, different routes of administration may be employed, such as, subcutaneous bolus and intravenous bolus. In yet other embodiments, the amount of interferon-α may be changed, such as, administering interferon-α at a different dosing rate or different concentration. The dose may be varied at any time during the therapy, such as hours, days, weeks or even months after commencement of therapy.

The terms “pharmacodynamic models” and “pharmacodynamic parameters” as used herein also include viral kinetic models and viral kinetic parameters. Various models to estimate Hepatitis C viral kinetics have been developed, and may be used for methods described herein. Examples of suitable viral kinetic models include, but are not limited to, models disclosed in the following references: International Application Number PCT/US2009/038617, the contents of which are incorporated by reference; Alan S. Perelson, et al. (2005). “New kinetic models for the hepatitis C virus.” Hepatology 42(4): 749-754; Andrew H Talal, et al. (2006). “Pharmacodynamics of PEG-IFN α Differentiate HIV/HCV Coinfected Sustained Virological Responders from Nonresponders.” Hepatology 43(5): 943-953; Dahari, H., A. Lo, et al. (2007). “Modeling hepatitis C virus dynamics: liver regeneration and critical drug efficacy.” J Theor Biol 247(2): 371-81; Dahari, H., R. M. Ribeiro, et al. (2007). “Triphasic decline of hepatitis C virus RNA during antiviral therapy.” Hepatology 46(1): 16-21. Dixit, N. M., J. E. Layden-Almer, et al. (2004). “Modelling how ribavirin improves interferon response rates in hepatitis C virus infection.” Nature 432(7019): 922. Neumann, A. U., N. P. Lam, et al. (1998). “Hepatitis C viral dynamics in vivo and the antiviral efficacy of interferon-alpha therapy.” Science 282(5386): 103-7. Powers, et al. (2003). “Modeling viral and drug kinetics: hepatitis C virus treatment with pegylated interferon alfa-2b.” Semin Liver Dis 23 Suppl 1: 13-18. Powers, K. A., R. M. Ribeiro, et al. (2006). “Kinetics of hepatitis C virus reinfection after liver transplantation.” Liver Transpl 12(2): 207-16; Bonate, P. L. (2006). Pharmacokinetic-Pharmacodynamic Modeling and Simulation. New York, Springer Science&Business Media; Gabrielsson, J. and D. Weiner (2000); and Pharmacokinetic and Pharmacodynamic Data Analysis: Concepts and Applications. Stockholm, Swedish Pharmaceutical Press.

In typical embodiments of the invention, efficacy is defined as the ability of a drug to produce a desired therapeutic effect or a clinical outcome. The efficacy of interferon-α treatment may be described in terms of overall efficacy (ε), in terms of blocking virion production (εₚ) or in terms of reducing new infections (η). Efficacy may also indicate the rate of sustained virological response, early virological response, rapid virological response, and so forth.

The term “actual efficacy” means an efficacy achieved by administering to a patient an interferon dose. The actual efficacy may be calculated from the clinical outcome, such as interferon serum concentration or viral load data. The term “critical efficacy” means a critical value of efficacy such that for efficacies above the critical value the virus is ultimately cleared in a significant number of patients, while for efficacies below it, virus is not cleared in a significant number of patients. In this context, those of skill in the art understand that different patients are observed to respond differently to identical HCV therapeutic regimens and that no single regimen will produce identical results in all patients. The term “desired efficacy” means a value of efficacy that is estimated to result in a desired clinical outcome including, for example, desired value of, rate of change of, or trend of change in viral load, number of infected target cells, number of uninfected target cells and so forth. The desired efficacy is typically set to maximize the difference between the actual efficacy and the critical efficacy while minimizing the side effects on the patient.

Efficacy of interferon may be varied by varying the dosing rate of interferon-α. The term “dosing rate” as contemplated herein depends on a quantity of interferon-α delivered over time, and may be optimized by changing interferon's administration rate or interferon's concentration. In addition, the term “dosing rate” as used herein may also depend on a quality of interferon-α, and may be changed by switching to a more potent interferon-α formulation. The dosing rate may be varied rapidly or gradually from one constant rate to another, or according to an approximately sinusoidal function.

Those of skill in this art understand that although some pK or pD parameters may be determined in a matter of hours or days, determining other parameters may require data taken over longer periods of time such as weeks or months. In addition, many of the pK and pD parameters as well as the structure and complexity of the model may change during the therapy. Accordingly, the blood samples for determination of pK and pD parameters may be taken throughout the therapy. More specifically, the samples may be taken from 0 to at least 48 weeks after commencement of therapy. Typically, the blood samples may be taken more frequently around the peak and less frequently around the tail. Furthermore, the duration of sampling may also depend on the type of interferon-α used as well as on the individual's response to therapy. In one specific embodiment, the samples for determination of may be taken at 0, 2, 4, 6, 8, 10, 12, 16, 20, 24, 36, 38, 40, 42, 44, 46, 48, 52, 56, 60, 72, 96, 120, 144, and 168 hours during week 1, and then at week 2, 4, 8, 16, 24, 36 and 48. In another embodiment, samples are taken every week up to week 48 or 72. Data for concentration and viral load may be obtained according to the same or different schedule. It will also be understood that samples may be taken more frequently in order to provide adequate feedback to the controller, and these samples may also be used to determine or optimize the pK and pD parameters.

One of ordinary skill in the art can appreciate that in various embodiments of the invention, the dosing rates may be dependent or independent of each other. If dependent, the dosing of the first stage may be set to fall between at least 5 to 95%, or at least 20% and 80%, or at least 20 and 50%, or at least 25% of the dosing rate of the second stage (dosing rate resulting in a higher efficacy). The second stage may last for the remainder of the therapy or, alternatively, may be followed by one or more additional stages. The efficacy during the additional stages may be higher or lower than the efficacy during the second stage. However, in the typical embodiment, the second stage of the therapy would always provide a higher level of the actual efficacy as compared to the actual efficacy during the first stage of the therapy.

### Exemplary Computer System Embodiments of the Invention

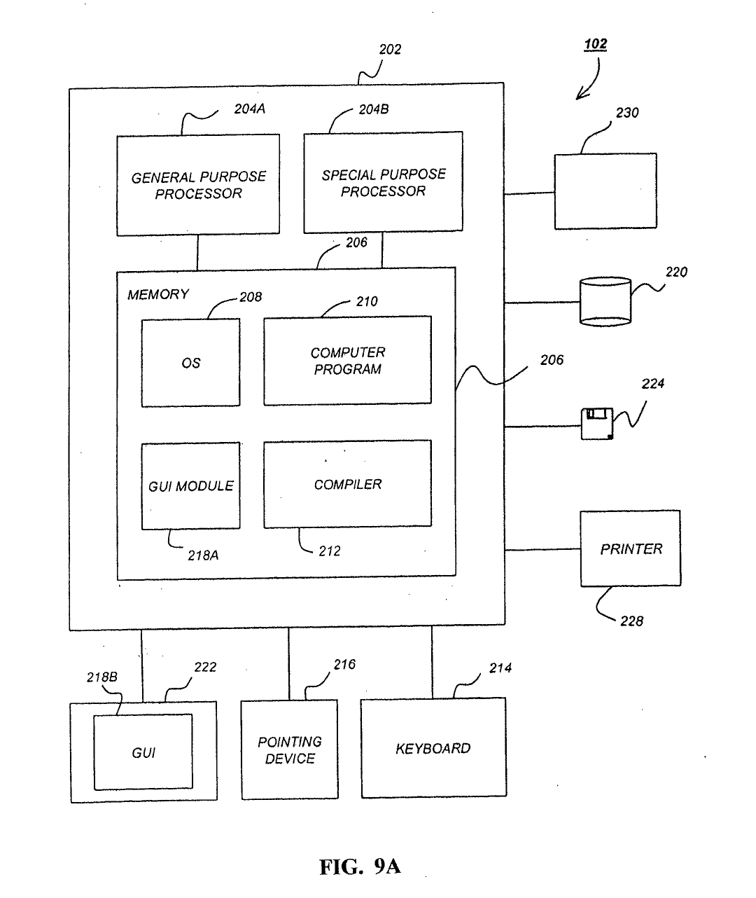

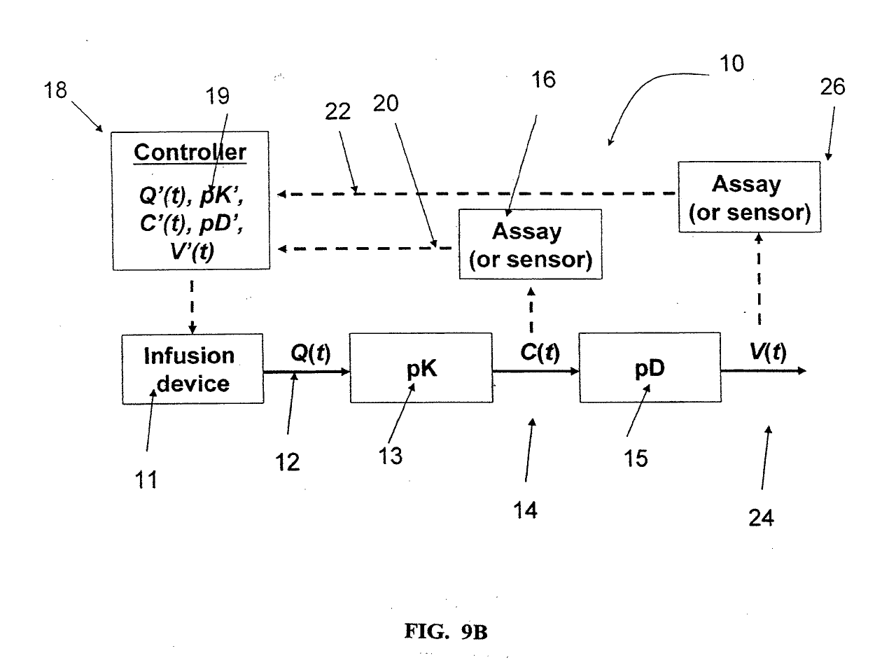

Embodiments of the invention disclosed herein can be performed for example, using one of the many computer systems known in the art (e.g. those associated with medication infusion pumps). FIG. 9A illustrates an exemplary generalized computer system 202 that can be used to implement elements the present invention, including the user computer 102, servers 112, 122, and 142 and the databases 114, 124, and 144. The computer 202 typically comprises a general purpose hardware processor 204A and/or a special purpose hardware processor 204B (hereinafter alternatively collectively referred to as processor 204) and a memory 206, such as random access memory (RAM). The computer 202 may be coupled to other devices, including input/output (I/O) devices such as a keyboard 214, a mouse device 216 and a printer 228.

In one embodiment, the computer 202 operates by the general purpose processor 204A performing instructions defined by the computer program 210 under control of an operating system 208. The computer program 210 and/or the operating system 208 may be stored in the memory 206 and may interface with the user 132 and/or other devices to accept input and commands and, based on such input and commands and the instructions defined by the computer program 210 and operating system 208 to provide output and results. Output/results may be presented on the display 222 or provided to another device for presentation or further processing or action. In one embodiment, the display 222 comprises a liquid crystal display (LCD) having a plurality of separately addressable liquid crystals. Each liquid crystal of the display 222 changes to an opaque or translucent state to form a part of the image on the display in response to the data or information generated by the processor 204 from the application of the instructions of the computer program 210 and/or operating system 208 to the input and commands. The image may be provided through a graphical user interface (GUI) module 218A. Although the GUI module 218A is depicted as a separate module, the instructions performing the GUI functions can be resident or distributed in the operating system 208, the computer program 210, or implemented with special purpose memory and processors.

Some or all of the operations performed by the computer 202 according to the computer program 110 instructions may be implemented in a special purpose processor 204B. In this embodiment, the some or all of the computer program 210 instructions may be implemented via firmware instructions stored in a read only memory (ROM), a programmable read only memory (PROM) or flash memory in within the special purpose processor 204B or in memory 206. The special purpose processor 204B may also be hardwired through circuit design to perform some or all of the operations to implement the present invention. Further, the special purpose processor 204B may be a hybrid processor, which includes dedicated circuitry for performing a subset of functions, and other circuits for performing more general functions such as responding to computer program instructions. In one embodiment, the special purpose processor is an application specific integrated circuit (ASIC).

The computer 202 may also implement a compiler 212 which allows an application program 210 written in a programming language such as COBOL, C++, FORTRAN, or other language to be translated into processor 204 readable code. After completion, the application or computer program 210 accesses and manipulates data accepted from I/O devices and stored in the memory 206 of the computer 202 using the relationships and logic that was generated using the compiler 212. The computer 202 also optionally comprises an external communication device such as a modem, satellite link, Ethernet card, or other device for accepting input from and providing output to other computers.

In one embodiment, instructions implementing the operating system 208, the computer program 210, and the compiler 212 are tangibly embodied in a computer-readable medium, e.g., data storage device 220, which could include one or more fixed or removable data storage devices, such as a zip drive, floppy disc drive 224, hard drive, CD-ROM drive, tape drive, etc. Further, the operating system 208 and the computer program 210 are comprised of computer program instructions which, when accessed, read and executed by the computer 202, causes the computer 202 to perform the steps necessary to implement and/or use the present invention or to load the program of instructions into a memory, thus creating a special purpose data structure causing the computer to operate as a specially programmed computer executing the method steps described herein. Computer program 210 and/or operating instructions may also be tangibly embodied in memory 206 and/or data communications devices 230, thereby making a computer program product or article of manufacture according to the invention. As such, the terms “article of manufacture,” “program storage device” and “computer program product” as used herein are intended to encompass a computer program accessible from any computer readable device or media.

Of course, those skilled in the art will recognize that any combination of the above components, or any number of different components, peripherals, and other devices, may be used with the computer 202. Although the term “user computer” is referred to herein, it is understood that a user computer 102 may include portable devices such as medication infusion pumps, analyte sensing apparatuses, cellphones, notebook computers, pocket computers, or any other device with suitable processing, communication, and input/output capability.

FIG. 9B presents a specific illustrative embodiment system 10 for performing methods disclosed herein. The interferon-α may be administered at a dosing rate Q(t) 12 from an infusion device 11 including, but not limited to, a pump, a depot, an infusion bag, or the like. Once the therapy is commenced, the interferon-α serum concentration 14, represented as C(t), may be determined by sampling a patient's blood by assay or sensor 16, and communicated to a controller 18, as represented by a concentration feedback loop 20. In addition to or instead of loop 20, the system 10 may also include a viral load feedback loop 22. According to the loop 22, patient's viral load 24, represented as V(t), may be determined by sampling patient's blood by assay or sensor 26 and may be communicated to the controller 18. Based on C(t), V(t) or both, controller 18 may calculate the dosing rate 12, which may then be adjusted if necessary either automatically by the controller or manually by an individual administering the therapy. In addition, patient-specific pK parameters 13 and pD parameters 15 may be determined from this data. Although the controller 18 may be a conventional process controller such as a PID controller, one can also utilize an adaptive model predictive process controller or model reference adaptive control. In general, a model predictive controller may be programmed with mathematical models of a “process” to predict “process” response to proposed changes in the inputs. These predictions are then used to calculate appropriate control actions. In response to control actions, the model predictions are continuously updated with measured information from the “process” to provide a feedback mechanism for the controller. In addition, the mathematical models may be continuously optimized to match the performance of the “process.”

In the system shown in FIG. 9B, the controller 18 may be programmed with patient-specific pK or pD parameters, population or subpopulation averages, or a combination thereof together with pharmacokinetic and pharmacodynamic models to calculate the dosing rate necessary to achieve desired clinical outcome. During the therapy, the controller continuously processes the data received from the feedback loops to optimize the dosing rate based on a patient's response to the therapy. In some embodiments, the controller 18 may also manipulate the pharmacokinetic and pharmacodynamic parameters, as well as the mathematical models based on concentration and viral load data to adopt or customize the models for individual patients and specific conditions.

In FIG. 9B, the controller 18 may use patient-specific pharmacokinetic or pharmacodynamic parameters, population or subpopulation averages, or combination thereof together with pharmacokinetic, pharmacodynamic, or viral kinetic models to calculate the dosing rate for desired efficacy based on C(t), V(t) or both. In FIG. 9B, pK refers to the physical pharmacokinetic system of a real patient. On the other hand, the parameter pK′ 19 refers to the pharmacokinetic model and parameter values used by the controller to describe pK, and which may be drawn from the real patient, population, or subpopulation averages. Similar notation is used for pD, C, V and Q.

In an embodiment of a system 10 having the loop 22 only, a given patient is assumed to have a set of individual pharmacokinetic parameters, represented as pK, and thus actual efficacy may be represented as a function of concentration, which is a function of the dosing rate Q(t). The controller 18 may use pharmacokinetic and pharmacodynamic models to calculate the suitable dosing rate for desired efficacy based on the concentration or other physiological characteristic data. Such models are known and are disclosed in, for example, Bonate, P. L. (2006). Pharmacokinetic-Pharmacodynamic Modeling and Simulation. New York, Springer Science&Business Media; Andrew H Talal, et al. (2006). “Pharmacodynamics of PEG-IFN αDifferentiate HIV/HCV Coinfected Sustained Virological Responders from Nonresponders.” Hepatology 43(5): 943-953′ Gabrielsson, J. and D. Weiner (2000). Pharmacokinetic and Pharmacodynamic Data Analysis: Concepts and Applications. Stockholm, Swedish Pharmaceutical Press.

### Therapeutic Regimens Adapted to Aspects of HCV Biolology

Typically, HCV RNA levels exhibit a biphasic or triphasic decline in response to therapy. In a biphasic response, viral load rapidly declines during the first phase, and gradually declines during the second phase. In a triphasic response, a rapid initial decline in the viral load is followed by “shoulder phase”—in which viral load decays slowly or remains constant—and a third phase of resumed viral decay. See Dahari, H., A. Lo, et al. (2007). “Modeling hepatitis C virus dynamics: liver regeneration and critical drug efficacy.” J Theor Biol 247(2): 371-81. (hereinafter the “Dahari, 2007 reference”). In addition, some patients may exhibit a more complex pattern such as for example, a rebound in viral load after the first stage, or a change in the rate of decline in the middle of the second phase. Throughout this application, the term “phase” is used to refer to changes in viral load kinetics. On the other hand, the term “stage” is used to refer to changes in the dosing rate or efficacy. The phases and stages may or may not correspond to one another. In certain embodiments of the invention, one can maintain interferon-α (or other agent) efficacy at different levels, e.g. administer interferon-α at different dosing rates, at different phases of HCV RNA decline (see, e.g. International Publication No. WO 2009/046369, the contents of which are incorporated by reference).

Embodiments of the invention can further modulate specific parameters of a therapeutic regimen depending upon when the different phases of the viral life cycle occur in order to, for example, change the dosing rate of interferon. The term “dosing rate” as contemplated herein depends on a quantity of interferon-α delivered over time, and may be optimized by changing interferon's administration rate or interferon's concentration. In addition, the term “dosing rate” as used herein may also depend on a potency of interferon, and may be changed by switching to a more potent interferon-α formulation. The dosing rate may be varied rapidly or gradually from one constant rate to another, or according to an approximately sinusoidal function. In some embodiments, it may be advantageous to increase the dosing rate gradually, i.e., according to an approximated ramp function, in order to minimize adverse side effects by allowing the patient to acclimate to a higher dosing rate. Alternatively, especially if a patient is tolerant to interferon, it may be desirable to increase the dosing rate rapidly, i.e. according to an approximated step function, in order to maximize time at a higher dosing rate.

The patient-specific treatment regimens described herein provide for optionally measuring such patients' parameters as the baseline viral load or other parameters associated with Hepatitis C virus, which are described in more detail below. The regimen then provides for administration of interferon-α at a dosing rate preferably calculated to avoid severe side effects typically associated with interferon-α therapy. The patient may then be tested for the interferon-α serum concentration or the viral load or any other relevant parameters known to those of ordinary skill in the art to infer the actual efficacy. Based on the results of these tests and respective comparison of the baseline values, actual efficacy and critical efficacy may be estimated. Critical efficacy may be estimated from a patient's response to the initial dosing rate using various viral kinetics models. Then, the initial interferon-α dosing rate is adjusted to a second dosing rate where the actual efficacy is greater than or equal to the estimated critical efficacy. This process can be repeated as necessary for the duration of the therapy.

The duration of stages of a therapeutic regimen may be defined in terms of time or in terms of decline in the viral load. In some embodiments, the therapeutic regimen may be concluded when a patient's viral load stays at 10² International Units per Milliliter (IU/mL) or less, or 10² RNA copies/mL or less for at least 4 weeks, or at lowest detection limit of the assay for 4 weeks. By way of non-limiting example, in embodiments where the interferon-αis administered in a first therapeutic regimen, the first stage may last for at least 1 to at least 120 days, typically between at least 21 and at least 35 days, and optionally at least 28 days. In various embodiments, the second stage may last between at least 0 and at least 30 days, for example between at least 14 and at least 30 days. In other embodiments, the second stage may be followed by at least one more stage with an increased or decreased efficacy for the total treatment time of at least 24 weeks or at least 48 weeks. Alternatively, the initial stage may last until a 1-log or a 2-log reduction in viral load is measured. After the initial stage, the dosing rate may be increased and kept constant for the remainder of the therapy, or may be adjusted at least once again.

By way of non-limiting example, in embodiments where the interferon-α is administered at a high dosing rate, the first stage may last for at least 3 to at least 5 weeks, and typically for at least 4 weeks. In other embodiments, the first stage may last until HCV RNA level is between about the lower detection limit of the employed assay and 10⁷ IU/mL, 10 IU/mL and 10⁷ IU/mL, about 100 IU/mL and 10⁷ IU/mL, or about 10³ IU/mL and 10⁷ IU/mL Typically, the detection limit of the assay is about 10 to 100 IU/mL In yet other embodiments, the first stage may last until a 2-log reduction, a 3-log reduction, or a 4-log reduction in the viral load is achieved. The second stage may last for about 42 to 52 weeks, typically for at least 48 weeks. Alternatively, the second stage may last until HCV RNA is equal to or less than about 10² IU/mL, 10 copies/mL, or stays below the detection limit of the employed assay for about 4 weeks. The dosing rate may also be reduced multiple times, such as, for example, at 2 log reduction, then at 3 log reduction, and then at a 4 log reduction in HCV RNA levels for the remainder of the therapy.

In yet other embodiments, the duration of stages may be defined in terms of ratio of infected target cells to uninfected target cells. In one embodiment, the duration of stages may be defined in terms of ratio of infected target cells to uninfected target cells. It has been shown that not all hepatocytes (liver cells) may be intrinsically susceptible to hepatitis virus infection. On the contrary, cells other than hepatocytes, i.e. cells other than the ones that reside in the liver, may be susceptible to hepatitis virus infection. See Powers, K. A., R. M. Ribeiro, et al. (2006). “Kinetics of hepatitis C virus reinfection after liver transplantation.”Liver Transpl 12(2): 207-16. Accordingly, the term “target cells” means cells that are susceptible to hepatitis virus infection regardless of whether they are hepatocytes or other cell types.

### Systems for the Administration of Agents Such as Interferon:

In the therapeutic regimens described herein, therapeutic agents (e.g. interferon-α) can be administered in a substantially continuous manner. The term “substantially continuous manner” as contemplated herein means that the dosing rate is constantly greater than zero during the periods of administration. The term includes embodiments when the drug is administered at a steady rate, e.g. via a continuous infusion apparatus. In some embodiments, interferon-α may be administered only in a substantially continuous manner throughout the entire treatment period. In other embodiments, these manners of interferon-α administration may be combined during the same stage or altered during different stages of the treatment.

In certain embodiments of the invention, the therapeutic agent is administered in a “substantially continuous manner”. Typically the therapeutic agent is administered in a substantially continuous manner via a continuous infusion pump, for example a pump typically used to administer insulin to diabetic patient. Suitable types of pumps include, but are not limited to, osmotic pumps, interbody pumps, infusion pumps, implantable pumps, peristaltic pumps, other pharmaceutical pumps, or a system administered by insertion of a catheter at or near an intended delivery site, the catheter being operably connected to a pharmaceutical delivery pump. It is understood that such pumps can be implanted internally (e.g. into a patient's abdominal (peritoneal) cavity) or worn externally (e.g. clipped to belt loop) as appropriate. Typical methods of the invention employ a programmable pump for the methods described herein.

When selecting a suitable pump, a number of characteristics need to be considered. These characteristics include, but are not limited to, biocompatibility (both the drug/device and device/environment interfaces), reliability, durability, environmental stability, accuracy, delivery scalability, flow delivery (continuous vs. pulse flow), portability, reusability, back pressure range and power consumption. While biocompatibility is always an important consideration, other considerations vary in importance depending on the device application. A person with ordinary skill in the art is capable of selecting an appropriate pump for the methods described herein.

A variety of external or implantable pumps may be used to administer the interferon. One example of an external pump is Medtronic MiniMed® pump and one example of a suitable implantable pump is Medtronic SynchroMed® pump, both manufactured by Medtronic, Minneapolis, Minn. In these pumps, the therapeutic agent is pumped from the pump chamber and into a drug delivery device, which directs the therapeutic agent to the target site. The rate of delivery of the therapeutic agent from the pump is typically controlled by a processor according to instructions received from the programmer. This allows the pump to be used to deliver similar or different amounts of the therapeutic agent continuously, at specific times, or at set intervals between deliveries, thereby controlling the release rates to correspond with the desired targeted release rates. Typically, the pump is programmed to deliver a continuous dose of interferon-α to prevent, or at least to minimize, fluctuations in interferon-α serum level concentrations.

The interferon-α may be delivered subcutaneously, intramuscularly, parenterally, intraperitoneally, transdermally, or systemically. In specific embodiments, interferon-α may be delivered subcutaneously or for a systemic infusion. A drug delivery device may be connected to the pump and tunneled under the skin to the intended delivery site in the body. Suitable drug delivery devices include, but are not limited to, those devices disclosed in U.S. Pat. Nos. 6,551,290 and 7,153,292.

A wide variety of continuous infusion devices known in the art can be used to deliver one or more antiviral agents to a patient infected with HCV. Continuous interferon-α administration may for example be accomplished using an infusion pump for the subcutaneous or intravenous injection at appropriate intervals, e.g. at least hourly, for an appropriate period of time in an amount which will facilitate or promote a desired therapeutic effect. Typically the continuous infusion device used in the methods of the invention has the highly desirably characteristics that are found for example in pumps produced and sold by the Medtronic corporation. In illustrative embodiments of the invention, the cytokine is administered via an infusion pump such as a Medtronic MiniMed model 508 infusion pump. The Model 508 is currently a leading choice in insulin pump therapy, and has a long history of safety, reliability and convenience. Typically the pump includes a small, hand-held remote programmer, which enables diabetes patients to program cytokine delivery without accessing the pump itself.

Alternatively, continuous administration can by accomplished by, for example, another device known in the art such as a pulsatile electronic syringe driver (Provider Model PA 3000, Pancretec Inc., San Diego Calif.), a portable syringe pump such as the Graseby model MS1 6A (Graseby Medical Ltd., Watford, Herts England), or a constant infusion pump such as the Disetronic Model Panomat C-S Osmotic pumps, such as that available from Alza, may also be used. Since use of continuous subcutaneous injections allows the patient to be ambulatory, it is typical chosen for use over continuous intravenous injections.

Infusion pumps and monitors for use in embodiments of the invention can be designed to be compact (e.g. less than 15×15 centimeters) as well as water resistant, and may thus be adapted to be carried by the user, for example, by means of a belt clip. As a result, important medication can be delivered to the user with precision and in an automated manner, without significant restriction on the user's mobility or life-style. The compact and portable nature of the pump and/or monitor affords a high degree of versatility in using the device. As a result, the ideal arrangement of the pump can vary widely, depending upon the user's size, activities, physical handicaps and/or personal preferences. In a specific embodiment, the pump includes an interface that facilitates the portability of the pump (e.g. by facilitating coupling to an ambulatory user). Typical interfaces include a clip, a strap, a clamp or a tape.

A wide variety of formulations tailored for use with continuous infusion pumps are known in the art. For example, formulations which simulate a constant optimized dose injection, such as, but not limited to, short-acting unconjugated forms of interferon-α as well as long-acting interferon-α-polymer conjugates and various-sustained release formulations, are contemplated for use. Typical routes of administration include parenteral, e.g., intravenous, intradermal, intramuscular and subcutaneous administration. Solutions or suspensions used for parenteral, intradermal, or subcutaneous application can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. Regimens of administration may vary. Such regimens can vary depending on the severity of the disease and the desired outcome.

Following administration of a interferon-α, and/or ribavirin or other therapeutic agents to a person infected with HCV, the HCV burden in the individual can be monitored in various ways well known to the skilled practitioner familiar with the hallmarks of HCV infection. In the case of chronic hepatitis infection, a therapeutically effective amount of the drug may reduce the numbers of viral particles detectable in the individual and/or relieve to some extent one or more of the signs or symptoms associated with the disorder. For example, as disclosed in detail above, in order to follow the course of hepatitis replication in subjects in response to drug treatment, hepatitis RNA may be measured in serum samples by, for example, an rt-PCR procedure such as one in which a nested polymerase chain reaction assay uses two sets of primers derived from a hepatitis genome. Farci et al., 1991, New Eng. J. Med. 325:98-104. Ulrich et al., 1990, J. Clin. Invest., 86:1609-1614. Histological examination of liver biopsy samples may then be used as a second criteria for evaluation. See, e.g., Knodell et al., 1981, Hepatology 1:431-435, whose Histological Activity Index (portal inflammation, piecemeal or bridging necrosis, lobular injury and fibrosis) provides a scoring method for disease activity.

In another embodiment of the invention, an article of manufacture (e.g. a kit) containing materials useful for the treatment of HCV infection as described above is provided. The article of manufacture can comprise a container and a label. Suitable containers include, for example, continuous infusion pumps, infusion tubing sets, catheters, bottles, vials, syringes, and test tubes. The containers may be formed from a variety of materials such as glass or plastic. The container can hold a composition (e.g. cytokine or other therapeutic composition) which is effective for treating the condition (e.g. chronic hepatitis infection) and may have a sterile access port (for example the container may be an intravenous solution bag or a vial having a stopper pierceable by a hypodermic injection needle). The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The pharmaceutical compositions useful in the methods of the invention can be included in a container, pack, or dispenser together with instructions for administration. That result can be reduction and/or alleviation of the signs, symptoms, or causes of a disease or any other desired alteration of a biological system. For example, in a further embodiment of the invention, there are provided kits containing materials useful for treating pathological conditions with interferon. The article of manufacture comprises a container with a label. Suitable containers include, for example, bottles, vials, and test tubes. The containers may be formed from a variety of materials such as glass or plastic. The container holds a composition having an active agent which is effective for treating pathological conditions such as HCV infection. The active agent in the composition is typically interferon-α and/or ribavirin. The label on the container indicates that the composition is used for treating pathological conditions with interferon-α and/or ribavirin.

Those of skill in the art will understand that there are a variety of permutations of the disclosed methods, materials, systems, kits etc. One could for example, alter the dose or the duration of treatment depending upon aspects of HCV infection such an amount of virions eliminated and/or levels of multi-drug resistance observed in the patient.

Throughout this application, various journal articles, patents, patent applications, and other publications etc. are referenced to provide illustrations of the state of the art (e.g. U.S. Pat. No. (see, e.g. U.S. Pat. Nos. 6,172,046; 6,461,605; 6,387,365; and 6,524,570; U.S. Patent Application Nos.: 20060257365; 20070202078; 20050112093; 20050031586; 20030004119; and 20030055013 and Dahari, H., A. Lo, et al. (2007). “Modeling hepatitis C virus dynamics: liver regeneration and critical drug efficacy.” J Theor Biol 247(2): 371-81. Dahari, H., R. M. Ribeiro, et al. (2007). “Triphasic decline of hepatitis C virus RNA during antiviral therapy.” Hepatology 46(1): 16-21; and Dahari et al., Curr Hepat Rep. 2008; 7(3): 97-105.).

The present invention is not to be limited in scope by the embodiments disclosed herein, which are intended as single illustrations of individual aspects of the invention, and any that are functionally equivalent are within the scope of the invention. Further, even though the invention herein has been described with reference to particular examples and embodiments, it is to be understood that these examples and embodiments are merely illustrative of the principles and applications of the present invention. It is therefore to be understood that numerous modifications may be made to the illustrative embodiments and that other arrangements may be devised without departing from the spirit and scope of the present invention as defined by the following claims. Publications describing aspects of this technology include for example, U.S. Pat. Appln. Nos. 2005/0063949 and 2007/0077225; U.S. Pat. Nos. 6,172,046; 6,245,740; 5,824,784; 5,372,808; 5,980,884; published international patent applications WO 96/21468; WO 96/11953; Torre et al. (2001) J. Med. Virol. 64:455-459; Bekkering et al. (2001) J. Hepatol. 34:435-440; Zeuzem et al. (2001) Gastroenterol. 120:1438-1447; Zeuzem (1999) J. Hepatol. 31:61-64; Keeffe and Hollinger (1997) Hepatol. 26:101 S-107S; Wills (1990) Clin. Pharmacokinet. 19:390-399; Heathcote et al. (2000) New Engl. J. Med. 343:1673-1680; Husa and Husova (2001) Bratisl. Lek. Listy 102:248-252; Glue et al. (2000) Clin. Pharmacol. 68:556-567; Bailon et al. (2001) Bioconj. Chem. 12:195-202; and Neumann et al. (2001) Science 282:103; Zalipsky (1995) Adv. Drug Delivery Reviews S. 16, 157-182; Mann et al. (2001) Lancet 358:958-965; Zeuzem et al. (2000) New Engl. J. Med. 343:1666-1672; U.S. Pat. Nos. 5,985,265; 5,908,121; 6,177,074; 5,985,263; 5,711,944; 5,382,657; and 5,908,121; Osborn et al. (2002) J. Pharmacol. Exp. Therap. 303:540-548; Sheppard et al. (2003) Nat. Immunol. 4:63-68; Chang et al. (1999) Nat. Biotechnol. 17:793-797; Adolf (1995) Multiple Sclerosis 1 Suppl. 1:S44-S47. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention. However, the invention is only limited by the scope of the appended claims. All numbers recited in the specification and associated claims that refer to values that can be numerically characterized (e.g. the duration of a treatment, the concentration of a therapeutic compound etc.) can be modified by the term “about”.

### EXAMPLES

### Example 1

### General Therapeutic Regimens for the Continuous Administration of Interferon-α to Patients Infected with Hepatitis C Virus

A variety of art accepted therapeutic regimens for the treatment of HCV can be adapted for use in embodiments of the invention. For example, illustrative therapeutic regimens can comprise the use of an ambulatory infusion pump (e.g. MiniMed® model 508 micro infusion pump) for the continuous administration of interferon-α so as to maintain circulating levels of administered interferon-α above a certain threshold, for example a therapeutic regimen sufficient to maintain circulating levels of the interferon-α in the serum of the patient above a steady state concentration of at least 100, 200, 300, 400, 500, 600 or 700 pg/mL Such regimens can include, for example, administering 6, 9 or 12 MIU of IFN-α (e.g. Intron A®) per day for at least 1 week to at least 48 weeks, for example as discussed in detail in Example 2 below. Another illustrative regimen comprises the continuous administration of IFN-α 80,000 IU/kg/day for at least 1 week to at least 48 weeks. Another illustrative regimen comprises the continuous administration of IFN-α 120,000 IU/kg/day for at least 1 week to at least 48 weeks. Another illustrative regimen comprises the continuous administration of IFN-α 160,000 IU/kg/day for at least 1 to at least 48 weeks. Yet another illustrative regimen comprises the continuous administration of PegIntron 1.5 μg/kg SC weekly for at least 1 week to at least 48 weeks. Typically in such regimens, patents also receive oral ribavirin (e.g. 1000 mg/day if weight 75 kg; 1200 mg/day if weight >75 kg).

After patients have completed a first therapeutic regimen for a first time period (e.g. 1, 2, 3, 4, 5, 6 or 7 days, 1, 2, 3, or 4 weeks etc.), an analysis can be performed to observe for example, serum interferon-α levels, and/or the incidence of rapid and early virologic response (RVR and EVR, respectively) as well as safety/tolerability data and outcomes measures such as the illustrative measures disclosed herein. As discussed in Example 3 below, patient specific therapeutic regimen can then be designed based on the results of this analysis. For example, assuming that the analysis shows circulating levels of interferon-α to be within a target range, a patient can continue with an assigned treatment for the remainder of the treatment course. Alternatively, the patient can be administered a patient specific therapeutic regimen designed for example to increase serum interferon-α levels as compared to the first therapeutic regimen administered to the patient.

Embodiments of the invention further include systems such as those that comprise computer processors and the like coupled to a medication infusion pump and adapted to deliver interferon-α according to a specific therapeutic regimen. In some embodiments of the invention, the system includes one or more control mechanisms designed to modulate delivery of interferon-α, for example those that allow its delivery according to a predetermined infusion profile. For example, in some embodiments of the invention, a processor controls a therapeutic regimen that includes an infusion profile designed to take into account one or more characteristics of the patient (e.g. weight) and/or one or more characteristics of the hepatitis virus infecting the patient (e.g. genotype) and/or one or more characteristic of the therapeutic agent administered to the patient (e.g. its conjugation to a polyethylene glycol moiety). Optionally such profiles are selected from a plurality of predetermined infusion profiles. In certain embodiments of the invention, the system can be operably coupled to an input that provides information on the concentrations of exogenous IFN-α in a patient's serum (e.g. an input coupled to a sensor) and then uses the processor to modulate the dose of interferon-α administered to the patient so as to modulate the resulting in vivo serum concentrations up or down (e.g. so as to fall with an predetermined target ranges of concentrations).

### Example 2

### Clinical Studies on the Subcutaneous Continuous Infusion of interferon-α to HCV infected patients that fail To Respond to Conventional Therapeutic Regimens

### Background and Rationale

Treatment of chronic hepatitis C has shown an uneven success, with current clinical practices eliminating HCV in only about 50% of infected individuals. Consequently, there is a set of remaining factors such as viral genotype, which can for example reduce response rates in genotype 1. Optimal treatment of HCV genotype 1 patients with peginterferon-α and ribavirin has led to sustained virologic response (SVR) rates between 41-60% (see, e.g. Manns et al. Lancet 2001; 358:958-965; Fried et al. N Engl J Med 2002; 347:975-982; Hadziyannis et al. Ann Intern Med 2004; 140:346-355; and Zeuzem et al. C. J Hepatol 2005; 43:250-257). Improvement of these results and retreatment of previous nonresponders is considered to be the greatest challenge in this field.

Pegylation of interferon (IFN) alfa has improved the pharmacokinetic profile of conventional interferon-α by maintaining constant blood levels. This has enabled once-weekly IFN-α dosing and resulted in higher response rates. However, it has been shown that the IFN-α volume of distribution due to pegylation is considerably restricted (see, e.g. Zeuzem et al. Semin Liver Dis 2003; 23 Suppl 1:23-8), a factor which decreases biological activity and potentially decreases treatment efficacy. As disclosed herein, the continuous administration of IFN-α that has not been chemically modified via conjugation to a polyol can overcome these problems by providing sustained and constant levels of a fully potent IFN-α protein, one having a pharmacokinetic profile equivalent to endogenous interferon.

Aspects of continuous IFN-α infusion have been studied in chronic HCV patients. For example, a significant decrease in serum ALT was observed by Carreno et al. in 12 patients treated with continuous subcutaneous IFN-α2a (9 MIU) for 28 days (see, e.g. Carreno et al. J Med Virol 1992; 37:215-219). Irreversible side effects requiring dose modification were not observed. In a study from Schenker et al. IFN-α2b was administered by continuous subcutaneous infusion at a rate of 60,000 IU/h (10 million IU per week) for a period of 3 months in 7 patients previously treated with a standard course of IFN-α2b (see, e.g. Schenker et al. J Interferon Cytokine Res 1997; 17:665-670). Continuous infusion was tolerated well at the site of infusion. Moreover, systemic side effects were similar in type but were less intense compared to previous intermittent dosing.

For subcutaneous infusion Medtronic Inc. has a pump delivery system (MiniMed®, model 508 micro infusion pump) that has shown a good safety and tolerability profile in the continuous administration of daily 9 μg IFN alfacon-1 in 10 chronic HCV nonresponders (see, e.g. Tong et al. Hepatology 2003; 38:304 A). Preliminary data from the study in Tong et al. showed a substantial reduction of HCV RNA compared to other combination therapy at similar time-points. In addition, high dosages of both IFN-α and PEG-IFN-α have led to improved outcome in previous nonresponders (see, e.g. Vrolijk et al. J Viral Hepat 2003; 10:205-209; Marcellin et al. Hepatology 2005 42:657 A; and Cornberg et al. J Hepatol 2006; 44:291-301). Although side effects were more frequently seen in these studies, tolerability was not considered significantly lower compared to standard treatment.

Recent trials showed that optimal RBV dosages (approximately 15 mg/kg/day) are just as important as optimal IFN-α dosages in achieving SVR (see, e.g. Reddy et al. J Hepatol 2004; 40:149; and Shiffman et al. Hepatology 2005; 42:217 A). Significant higher SVR rates were seen in a recent study comparing fixed RBV dosing versus weight-based dosing (see, e.g. Jacobson et al. Hepatology 2005; 42:77 A). A fair proportion of nonresponders has been related to poor patient compliance, probably influenced by neuropsychiatric adverse effects, and by doctors adjusting or stopping medication on basis of cytopenia. SVR rates could have been higher if dose reductions by either adverse events or laboratory abnormalities had been prevented.

This example provides data from a clinical trial designed to examine the effects of the continuous administration of IFN-α to patients shown to be refractory to (PEG-)IFN-α/RBV combination therapy. For chronic hepatitis C patients refractory to previous (PEG-) IFN-α/RBV combination therapy we studied the continuous subcutaneous administration of high-dose IFN-α2b (Intron A®) for 48 weeks in combination with 15 mg/kg/day RBV (Rebetol®) and optimal management of side effects in order to maintain the highest possible dosages of both IFN-α2b and RBV for 48 weeks. As discussed below, we observe improved tolerability with continuous subcutaneous pump delivery of IFN-α2b compared to thrice weekly or daily subcutaneous injection of IFN-α2b, and increased antiviral activity and biologic potency due to sustained and higher levels of a fully potent interferon-α protein.

Aspects of this clinical trial study are disclosed below.

### Aims of the Study

This study uses a continuous infusion apparatus such as the MiniMed® model 508 micro infusion pump device to investigate therapeutic regimens which can optimize the dose, safety, and tolerability of continuous subcutaneous administration of high-dose IFN-α2b/ribavirin combination therapy in HCV (e.g. genotype 1) patients, such as those unresponsive to previous (peg)interferon/ribavirin combination therapy.

### Primary Objectives:

To study safety and tolerability of continuous subcutaneous infusion of high-dose IFN-α2b (serious adverse events, grade 4 NCI toxicity, percentage of patients completing treatment or reasons for dose adjustments).

### Secondary Objectives:

To study whether 48 weeks of continuous subcutaneous infusion of IFN-α2b at various (high) dosages in combination with daily oral ribavirin will lead to ETR and SVR in HCV genotype 1 patients, unresponsive to previous (peg)interferon/ribavirin combination therapy.
To study decline in viral load.
To study immunological response.

### Design of the Study

### Number of Patients

Thirty patients, with 10 patients in each treatment group

### Design (Type of Trial)

Monocenter, randomized, dose-finding study with three arms.

### Study Medication, Dosage and Duration

A total of thirty patients were randomized to receive 6, 9 or 12 MIU of IFN-α2b per day by continuous subcutaneous infusion for 48 weeks using the MiniMed® device. All groups received twice daily orally ribavirin with the following dosages: 65 kg: 1000 mg/day, >65-80 kg: 1200 mg/day, >80-100 kg: 1400 mg/day and >100 kg: 1600 mg/day. A follow-up is conducted at 72 weeks. Consequently, therapy was given for a total treatment period of 48 weeks. Post treatment follow-up lasted for 24 weeks.

### Outcome Measurements

### Primary Outcome:

Safety and tolerability of high-dose continuous subcutaneous infused IFN-α2b.

### Secondary Outcomes:

HCV RNA negativity by qualitative assay at week 48 (end of treatment, ETR) and 24 weeks after end of treatment (sustained virological response, SVR).
Biological activity of IFN-α2b represented as 2′5′-oligoadenylate synthetase (2′5′-OAS) and β₂-microglobulin activity.
Pharmacokinetics by IFN-α2b levels.
Viral decline during therapy.
Normalization of serum ALT at the end of therapy and at the end of follow-up.
Immunological response before, during and after treatment (frequency of dentritic cells (DCs) and regulatory T-cells (T_{regs}) in peripheral blood, function of DCs and HCV specific T-cell responses).
Quality of life and psychopathology by psychological assessment using SF-36 and SCL-90 questionnaires.

### Selection of Patients

### Patient Enrollment

A total of 30 eligible patients with chronic hepatitis C genotypes 1 or 4, unresponsive to conventional HCV antiviral therapy, were enrolled in the study.

### Inclusion Criteria:

Hepatitis C genotype 1 or 4, unresponsive to (peg)interferon-α/ribavirin therapy.
In the past, (peg)interferon-α or conventional interferon-α plus ribavirin combination therapy for at least 12 weeks and less than 2-log HCV RNA decrease at week 12, HCV RNA positivity at week 24, breakthrough during therapy or relapse after therapy.
At least 12 weeks between end of (peg)interferon/ribavirin therapy and start of high-dose IFN-α/ribavirin therapy.
Persistent indication for antiviral therapy such as persistently elevated serum ALT or histological evidence of continuing or progressive fibrosis.
Age 18-60 years.

### Exclusion Criteria:

Signs of progressive liver disease since end of previous therapy, beyond generally accepted criteria for HCV antiviral therapy:
   serum bilirubin >35 μ/mol/l, albumin <36 g/l, prothrombin time >4 sec or platelets<100,000/mm³.
   decompensated cirrhosis (defined as jaundice in the presence of cirrhosis, ascites, gastric bleeding, esophageal varices or encephalopathy).
Hepatic imaging (US, CT or MRI) with the evidence of hepatocellular carcinoma (hepatic imaging should be performed within 3 months prior to screening) or an alpha fetoprotein >20 ng/mL
Other acquired or inherited causes of liver disease that could explain liver disease activity.
Co-infection with hepatitis B virus or human immunodeficiency virus (HIV).
Other significant medical illness that might interfere with this study: significant cardiovascular, pulmonary or renal dysfunction, malignancy other than skin basocellular carcinoma in previous 5 years, immunodeficiency syndromes (e.g. HIV positivity, steroid therapy, organ transplants other than cornea and hair transplant).
History of a severe seizure disorder or current anticonvulsant use.
History of thyroid disease poorly controlled on prescribed medications.
Contra-indications for IFN-α and/or ribavirin:
   Severe psychiatric disorder, such as major psychoses, suicidal ideation, suicidal attempt and/or manifest depression during previous (peg)interferon-α therapy. Severe depression would include the following: (a) subjects who have been hospitalized for depression, (b) subjects who have received electroconvulsive therapy for depression, or (c) subjects whose depression has resulted in a prolonged absence of work and/or significant disruption of daily functions. Subjects with a history of mild depression may be considered for entry into the protocol provided that a pretreatment assessment of the subject's mental status supports that the subject is clinically stable and that there is ongoing evaluation of the patient's mental status during the study.
   Reactivation of immunological disorders during previous therapy.
   Visual symptoms related to retinal abnormalities.
   Pregnancy, breast-feeding or inadequate contraception.
   Thalassemia, spherocytosis.
Substance abuse, such as alcohol (≧80 gm/day) and I.V. drugs. If the subject has a history of substance abuse, to be considered for inclusion into the protocol, the subject must have abstained from using the abused substance for at least 2 years.
Any other condition which in the opinion of the investigator would make the patient unsuitable for enrollment, or could interfere with the patient participating in and completing the study.

### Study Medication, Supply, and Treatment of Patients

### Medication and Dosage Regimen

All patients received IFN-α2b by continuous subcutaneous infusion using the MiniMed® device. Patients were randomized to one of the following dosage regimes:

### 1) 12 MIU IFN-α2b per day.

### 2) 9 MIU IFN-α2b per day.

### 3) 6 MIU IFN-α2b per day.

Ribavirin is available in tablets of 200 mg and was weight-based dosed (approximately 15 mg/kg/day, see Table 1 below).

TABLE 1
RIBAVIRIN DOSING

| Weight | Ribavirin dosage | Morning dosage | Evening dosage |
|---|---|---|---|
| (kg) | (mg/day) | (# of tablets) | (# of tablets) |
| ≦65 | 1000 | 2 | 3 |
| >65-80 | 1200 | 3 | 3 |
| >80-100 | 1400 | 3 | 4 |
| >100 | 1600 | 4 | 4 |

### Adverse Events

Interferon alfa-2b

Most encountered adverse effects typically include headache, fatigue, fever/rigors, and myalgia. These are mostly mild in severity and tachyphylaxis was observed over the course of treatment. Important side effects include neuropsychiatric symptoms, such as lethargy, depression and emotional lability. Most of the significant changes in haematologic values (Hb, WBC, neutrophils and platelets) are mild or moderate in severity (grade 1 or 2) based on WHO/NCl criteria.

Other side effects include anorexia, erythema at injection side, diarrhea and induction of autoimmune disease (especially thyroiditis).

### Ribavirin

The most frequent reported side effects are: nausea, anorexia, dyspepsia, dizziness, rash, pruritus, skin eruptions, cough, nasal congestion, dyspnea. Most of these events are of mild to moderate severity in previous studies. The primary toxicity of ribavirin is hemolytic anemia, which is observed in approximately 13% of PEG-IFN-α/ribavirin treated patients. Fatal and nonfatal myocardial infarctions have been reported in patients with anemia caused by ribavirin.

### Dose Adjustment or Interruption

Management of common adverse events is generally achieved by dose reduction; however, in the case of life-threatening adverse events, identification of cardiac disease or development of cardiac dysfunction, pregnancy or failure to comply with the requirement for the practice of birth control, both IFN-α2b and RBV therapy must be discontinued permanently.

### Concomitant Medication

During treatment, paracetamol can be given to minimize the side-effects of IFN-α2b. The total daily dose of paracetamol should not exceed 4 gram. In case of anemia erytropoietin can be administered and blood transfusion is allowed. If depression or depressive symptoms occur, administration of selective serotonin reuptake inhibitors (SSRIs) is allowed. Concomitant medication, apart from drugs or therapies mentioned in the exclusion criteria, is permitted during the study, provided this pre-supposes no effect on the study outcome. The use of concomitant medication must be documented on the CRF (stating type, dosage and duration). If possible, existing concomitant medication should not be changed during the study.

### Management and Assessments of Patients

### Screening Assessments

Screenings were done within 28 days before start of therapy (day 0).

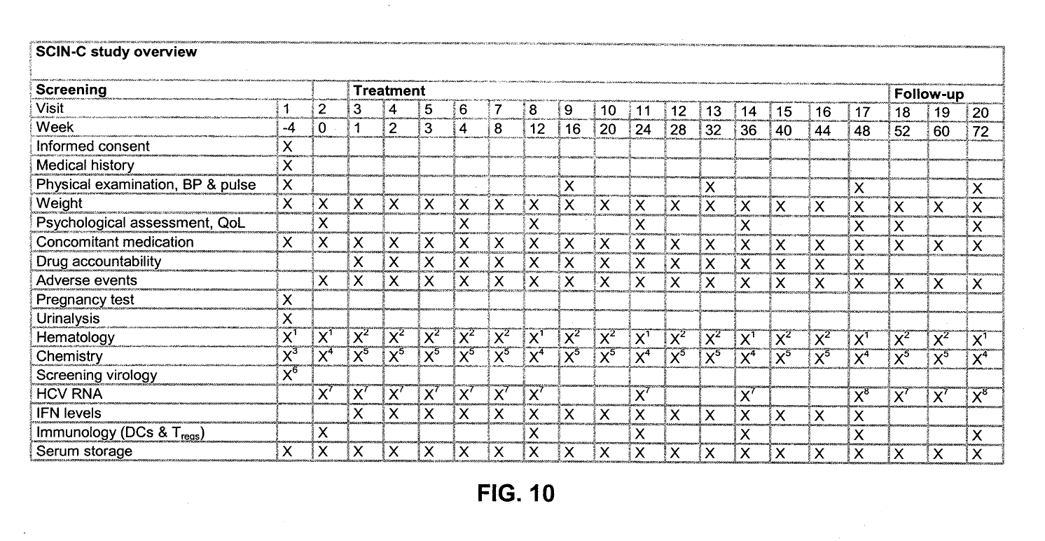

The following procedures were performed (see also the summary provided in FIG. 10):
Written informed consent.
Eligibility criteria check.
Physical examination, blood pressure, and pulse.
Medical history, concomitant medication.
General characteristics: initials, date of birth (dd/mm/yyyy), age (years), gender, weight (kg), height (cm), blood pressure (mmHg), heart rate, and ethnic background.
Pregnancy test in females between 18-50 years.
Lab hematology: Hb, platelets, leukocytes, absolute neutrophil count, prothrombin time
Lab chemistry: AST, ALT, total bilirubin, GGT, alkaline phosphatase, albumin, creatinine, TSH, LDH, Na, K, urea, amylase, CPK, glucose, α-fetoprotein, IgG, ANA, ASMA.
Lab virology: anti-HIV, HBsAg, anti-HBs, anti-HBc, HCV RNA by qualitative assay, HCV genotype.
Urinalysis via dipstick.
Plasma storage for future reference (6 mL).

### Baseline Assessments (Day 0)

Weight.
Lab hematology: Hb, platelets, leukocytes, absolute neutrophil count, prothrombin time.
Lab chemistry: AST, ALT, total bilirubin, GGT, alkaline phosphatase, albumin, creatinine, TSH.
Virology: HCV RNA by quantitative assay.
100 mL of blood (Na-heparin) will be taken for isolation of DCs and T_{regs}
2′5′-OAS and β₂-microglobulin levels.
Adverse events.
Concomitant medication.
Serum storage for future reference (6 mL).
Quality of life and psychopathology by psychological assessment.

### Treatment Phase

Physical examination, blood pressure and pulse (at week 16, 32, 48).
Weight (each visit).
Lab hematology:
   Each visit: Hb, platelets, leucocytes, absolute neutrophil count.
   At week 12, 24, 36, 48: also prothrombin time.
Lab chemistry:
   Each visit: AST, ALT.
At week 12, 24, 36, 48: also total bilirubin, GGT, alkaline phosphatase, albumin, creatinine, TSH.
Virology: HCV RNA:
   At week 1, 2, 3, 4, 8, 12, 24, 36, 48: quantitative assay.
   At week 48: qualitative assay (if negative by quantitative assay).
2′5′-OAS and β₂-microglobulin levels (each visit).
100 mL of blood (Na-heparin) will be taken for isolation of DCs and T_{regs} (at week 12, 24, 36, 48).
IFN-α2b levels (each visit).
Drug accountability (each visit).
Adverse events (each visit).
Concomitant medication (each visit).
Serum storage for future reference (6 mL) (each visit).
Quality of life and psychopathology by psychological assessment (at week 4, 12, 24, 36, 48).

### Follow-Up Phase

Physical examination, blood pressure and pulse (at week 72).
Weight (each visit).
Lab hematology:
   Each visit: Hb, platelets, leucocytes, absolute neutrophil count.
   At week 72: also prothrombin time.
Lab chemistry:
   Each visit: AST, ALT.
   At week 72: also total bilirubin, GGT, alkaline phosphatase, albumin, creatinine, TSH.
Virology: HCV RNA:
   At week 52, 60, 72: quantitative assay.
   At week 72: qualitatitve assay (if negative by quantitative assay).
2′5′-OAS and β₂-microglobulin levels (each visit).
100 mL of blood (Na-heparin) will be taken for isolation of DCs and T_{regs} (at week 72).
Adverse events (each visit).
Concomitant medication (each visit).
Serum storage for future reference (6 mL) (each visit).
Quality of life and psychopathology by psychological assessment (at week 52, 72).

### Statistics

The percentage of EVR and SVR in the three dosages regimes of continuous subcutaneous IFN-α2b therapy can be compared using Chi-Square test.

The log viral decline and pharmacokinetics over time can be analysed with non-linear regression applying repeated measurement analysis techniques.

ALT, biological activity, immunological response and quality of life assessment can be analysed with linear regression applying repeated measurement analysis.

Descriptive statistics will be used to assess safety and tolerability data. The percentage of adverse events (AEs), severe adverse events (SAEs) and dose reductions can be compared between all groups using Chi-Square test.

### I. Primary Analysis of Clinical Trial Data

### A. Background:

Pegylation of IFN-α is known to improve the PK profile with higher SVRs compared to standard IFN-α. The volume of distribution and biological activity, however, are substantially reduced. In this context, primary clinical data from the clinical trial provides evidence that the continuous exposure to therapeutic IFN-α levels not only prevents peaks associated with adverse events, but also troughs associated with subtherapeutic drug levels and viral breakthrough.

### B. Methods:

30 HCV genotype 1 (n=24) and 4 (n=6) patients received daily 6, 9 or 12MIU IFN alfa-2b (n=10 per group) by continuous subcutaneous administration using an infusion device designed for insulin infusion (Medtronic MiniMed 508) in combination with weight-based RBV (1000-1600 mg/day). Safety, tolerability, viral kinetics and pharmacokinetics were then assessed. Patients included in this study either had a prior history of non-responsiveness to therapy (n=20), relapse (n=7) or viral breakthrough (n=3) during previous PegIFN-α/RBV therapy. N=13 patients had cirrhosis at start of therapy. Patients negative for HCV RNA by TaqMan HCV Test (LLD <15 IU/mL) at week 24 were allowed to complete 48 weeks of therapy.

### C. Primary Results:

At week 4, a mean HCV RNA decline of 1.19 (95% CI 0.55-1.83), 1.21 (95% CI 0.38-2.04) and 2.67 (95% CI 2.38-2.97) log₁₀ IU/mL was seen with 6, 9 and 12MIU IFN-α/day, respectively (12MIU vs. 9MIU/6MIU, p<0.0001). IFN-α levels increased dose-dependently, reaching peak-levels between 48 hrs and week 1 followed by steady-state levels. Neopterin levels increased equally among the 3 groups between 48 and 96 hrs, with somewhat higher steady-state levels in the 12MIU group. HCV RNA negativity at week 24 was achieved in 2 (20%), 5 (50%) and 5 (50%), patients on 6, 9, and 12MIU IFN-α/day, respectively. AEs were mostly mild to moderate and typically IFN-α-related. Five patients experienced 6 SAEs including: community acquired pneumonia at week 10, diarrhea, dehydration, and fever at week 12, upper respiratory tract infection at week 6, injection site reaction at week 18 followed by hyperglycaemia induced seizure at week 21 (all 12MIU) and injection site reaction at week 12 (9MIU). SAEs led to temporary suspension of therapy in 3 patients and permanent discontinuation in 3; 4 of them had cirrhosis. No problems with regard to pump handling by patients were seen.

### D. Primary Analysis:

Continuous subcutaneous administration of IFN-α in difficult-to-treat patients showed high week 24 response rates at doses of 9 and 12 MIU/day. Daily 12 MIU IFN-α showed a significantly stronger HCV RNA drop at week 4 compared to lower doses. A good safety and tolerability profile was found. Understandably, typical interferon-related adverse events appeared more significant in the highest dose group of 12MIU IFN-α/day and in cirrhotics.

### 2. Characterization of Aspects of Primary Clinical Trial Data

This disclosure establishes some parameters important in treating hepatitis with interferon-α via continuous subcutaneous infusion.

The SCIN-C trial, conducted in the Netherlands in the city of Rotterdam at the Erasmus Medical Center was a three arm (treatment regimen) study with 10 subjects in each arm/regimen. The interferon-α dosages in the trial were 6 MIU, 9 MIU, and 12 MIU daily via pump with concomitant weight based oral ribavirin. The patients in the study are all previous therapy failures and are all Genotype 1 or 4. Previous therapy and certain subject specific data are in Table 2 below.

TABLE 2
SCIN-C Subject Data

| Subjects | Previous Peg/RBV | Genotype | Study Outcome | Remarks |
|---|---|---|---|---|
| 12 MIU | | | | |
| 1 | Non-response week 24 | 1 | Premature | SAE week 9: community |
| | | | discontinuation | acquired pneumonia |
| | | | week 9 | |
| 2 | Relapse | 1 | Non-response week 24 | |
| 3 | Non-response week 24 | 1 | Non-response week 24 | |
| 4 | Non-response week 12 | 1 | Completed week 48 | HCV RNA negative week 24 |
| 5 | HCV RNA positive | 1 | On treatment | HCV RNA negative week 24 |
| | at week 48, viral | | | |
| | breakthrough | | | |
| 6 | Non-response week 12 | 1 | Premature | Pt in detention at week 3 |
| | | | discontinuation | |
| | | | week 3 | |
| 7 | Relapse | 1 | On treatment | HCV RNA negative week 24 |
| 8 | Non-response week 24 | 1 | Premature | HCV RNA negative |
| | | | discontinuation | week 12, SAE week 21: |
| | | | week 21 | seizure due to hyperglycemia |
| 9 | Non-response week 24 | 4 | On treatment | HCV RNA negative week 12 |
| 10 | Relapse | 4 | On treatment | |
| 9 MIU | | | | |
| 1 | Relapse | 1 | Non-response week 24 | |
| 2 | Non-response week 24 | 1 | Non-response week 24 | |
| 3 | Non-response week 12 | 1 | Non-response week 24 | |
| 4 | HCV RNA positive | 1 | Premature | SAE week 12: injection site |
| | at week 48, viral | | discontinuation | reaction; pt withdrew consent |
| | breakthrough | | week 12 | |
| 5 | 18 week Peg/RBV, >2 | 1 | Completed week 48 | HCV RNA negative week 24 |
| | log 10 decline week 12 | | | |
| | (HCV RNA positive), stop | | | |
| | rx week 18 due to | | | |
| | suspected pneumonia | | | |
| 6 | Non-response week 12 | 1 | Premature | Pt withdrew informed consent: |
| | | | discontinuation | advise from her Russian GP |
| | | | week 4 | |
| 7 | Non-response week 12 | 4 | On treatment | HCV RNA negative week 24 |
| 8 | Non-response week 24 | 1 | On treatment | HCV RNA negative week 24 |
| 9 | Non-response week 24 | 4 | On treatment | HCV RNA negative week 12 |
| 10 | Non-response week 12 | 1 | On treatment | |
| 6 MIU | | | | |
| 1 | 36 week Peg/RBV, | 1 | Non-response week 24 | Lost to follow-up |
| | always detectable | | | |
| | HCV RNA, load | | | |
| | during therapy | | | |
| | unknown | | | |
| 2 | Non-response week 24 | 1 | Non-response week 24 | |
| 3 | Relapse | 1 | Non-response week 24 | Lost to follow-up |
| 4 | Non-response week 12 | 1 | Non-response week 24 | |
| 5 | Non-response week 24 | 4 | Non-response week 24 | |
| 6 | Non-response week 12 | 1 | Non-response week 24 | |
| 7 | Relapse | 1 | On treatment | HCV RNA negative week 24 |
| 8 | Non-response week 12 | 1 | Non-response week 24 | |
| 9 | Non-response week 24 | 4 | On treatment | HCV RNA negative week 12 |
| 10 | Non-response week 12 | 1 | On treatment | |

Based on data from this Table we have the following summary statistics: Genotype 1 24/30 (80%), Interferon-α non-responder week 12 10/30 (33%), HCV Positive week 24 12/30 (40%), Relapse/Rebound 8/30 (26.7%). In clinical practice it has been shown that non-responders at week 12 are the most difficult to retreat, while relapsers and rebounders are the least difficult to treat.

During the course of the study, blood levels of interferon-α were measured and pharmacokinetic and pharmacodynamic information from patients such as mean interferon-α and neopterin concentrations that result from the therapeutic regimens used in this trial are shown for example in FIG. 1. The data shown in FIG. 1 provides evidence there is a strong dose response to interferon-α administered following the disclosed therapeutic regimens. The data shown in FIG. 1 further provides evidence that delivering higher concentrations of interferon-α following the therapeutic regimens used in this trial leads to correspondingly higher concentrations of interferon-α that are sustained in vivo.

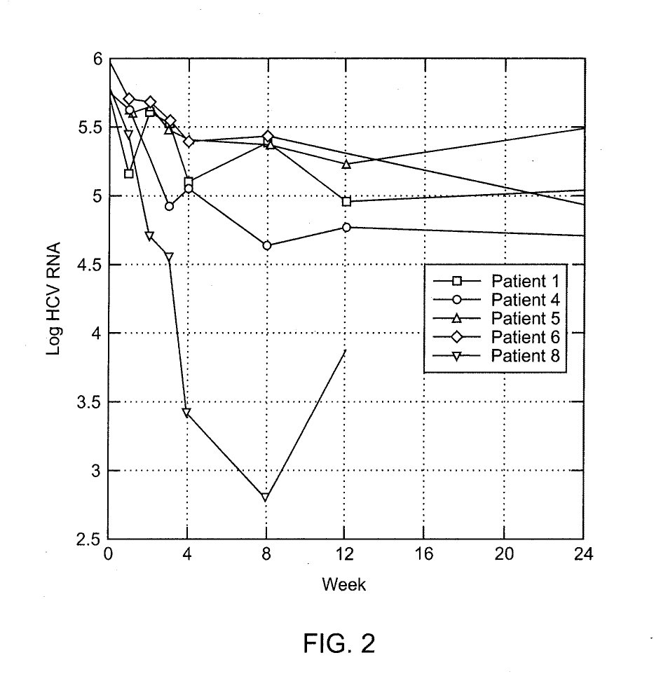

FIG. 2 shows viral decay curves in patients that are severely interferon-α resistant (and these patients are consequently difficult to treat). As shown in FIG. 2, in the 6 MIU/day treatment group there were 5 subjects that showed significant resistance. Of these 5 subjects, only patient 8 showed a robust response at week 8 with subsequent rebound. In previous therapy all of these 5 subjects were either therapy failures at week 12 or week 24. Five subjects with more robust HCV declines are shown in FIG. 3.

FIG. 3 provides data showing a robust response in the 6 MIU treatment group. In the lowest dose treatment arm, patients 2 and 3 both were viral negative by quantitative RNA testing at week 24 but tested positive by qualitative highly sensitive testing at week 24 and are out of the study. The other subjects continued in the study.

In the 9 MIU per day group, there were 4 subjects who were interferon-α resistant.

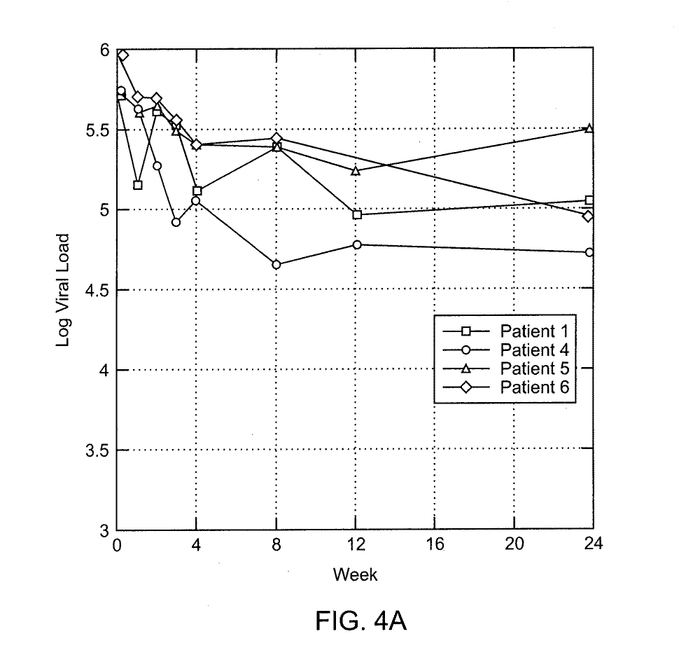

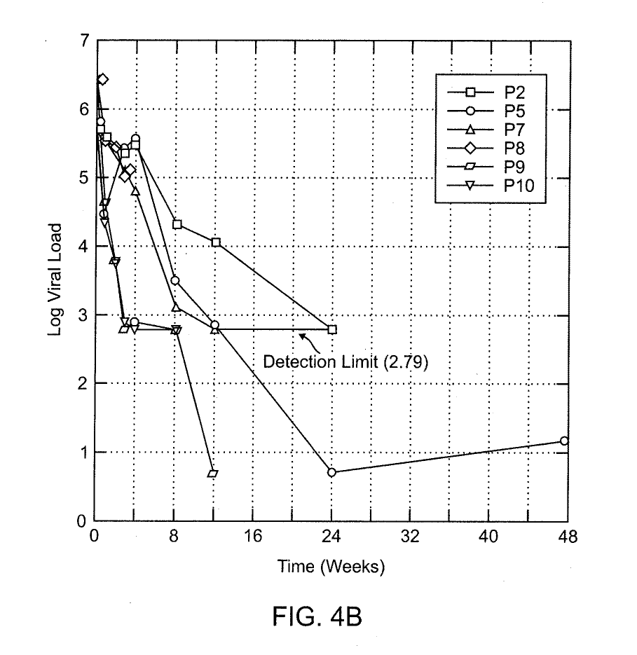

This data is shown in FIG. 4A. Of more interest is that 6 of the 10 subjects in the 9 MIU per day group showed a robust response. The data is shown in FIG. 4B. Of these subjects in the 9 MIU per day group, all were previously interferon-α resistant in PEG therapy.

In the 12 MIU per day treatment group there were no interferon-α resistant subjects.

Three subjects have withdrawn but 9 have shown a reasonably robust response to date as shown in FIG. 5.

The viral kinetic summary data is shown in Tables 3-5 below.

TABLE 3
6 MIU/Day Treatment Group

| | Previous | | | 4 Week | 12 Week | 24 Week |
|---|---|---|---|---|---|---|
| Subject | Therapy | Genotype | Outcome | Data/RVR | Data/EVR | Data/VN |
| 1 | Always | 1 | Non- | 0.6/N | 0.75/N | 0.67/N |
| | detectable | | response | | | |
| | HCV RNA | | week 24 | | | |
| 2 | Non- | 1 | Non- | 2.64/N | 3.54/Y | 3.54/N |
| | response | | response | | | |
| | week 24 | | week 24 | | | |
| 3 | Relapse | 1 | Non- | 1.15/N | 2.94/Y | 2.94/N |
| | | | response | | | |
| | | | week 24 | | | |
| 4 | Non- | 1 | Non- | 0.69/N | 0.97/N | 1.03/N |
| | response | | response | | | |
| | week 12 | | week 24 | | | |
| 5 | Non- | 4 | Non- | 0.18/N | 0.36/N | 0.1/N |
| | response | | response | | | |
| | week 24 | | week 24 | | | |
| 6 | Non- | 1 | Non- | 0.57/N | No Data | 1.03/N |
| | response | | response | | | |
| | week 12 | | week 24 | | | |
| 7 | Relapse | 1 | On | 2.07/N | 2.07/Y | 2.79/Y |
| | | | treatment | | | |
| 8 | Non- | 1 | Non- | 2.38/N | 1.92/N | No Data |
| | response | | response | | | |
| | week 12 | | week 24 | | | |
| 9 | Non- | 4 | On | 1.4/N | No Data | No Data |
| | response | | treatment | | | |
| | week 24 | | | | | |
| 10 | Non- | 1 | On | 1.06/N | 2.74/Y | No Data |
| | response | | treatment | | | |
| | week 12 | | | | | |

TABLE 4
9 MIU/Day Treatment Group

| | Previous | | | 4 Week | 12 Week | 24 Week |
|---|---|---|---|---|---|---|
| Subject | Therapy | Genotype | Outcome | Data/RVR | Data/EVR | Data/VN |
| 1 | Relapse | 1 | Non- | 0.71/N | 0.96/N | 1.04/N |
| | | | response | | | |
| | | | week 24 | | | |
| 2 | Non- | 1 | Non- | 0.24/N | 1.65/N | 2.91/N |
| | response | | response | | | |
| | week 24 | | week 24 | | | |
| 3 | Non- | 1 | Non- | −0.03/N | −0.02/N | 0.06/N |
| | response | | response | | | |
| | week 12 | | week 24 | | | |
| 4 | HCV RNA | 1 | Premature | 0.93/N | No Data | No Data |
| | positive at | | discontinuation | | | |
| | week 48, | | week 12 | | | |
| | viral | | | | | |
| | breakthrough | | | | | |
| 5 | 18 week | 1 | Completed | 0.26/N | 2.97/Y | 5.11/Y |
| | Peg/RBV, | | week 48 | | | |
| | >2 log10 | | | | | |
| | decline | | | | | |
| | week 12 | | | | | |
| | (HCV RNA | | | | | |
| | positive), | | | | | |
| 6 | Non- | 1 | Premature | 0.75/N | No Data | No Data |
| | response | | discontinuation | | | |
| | week 12 | | week 4 | | | |
| 7 | Non- | 4 | On treatment | 1.61/N | 3.62/Y | 3.62/Y |
| | response | | | | | |
| | week 12 | | | | | |
| 8 | Non- | 1 | On treatment | 1.29/N | No Data | No Data |
| | response | | | | | |
| | week 24 | | | | | |
| 9 | Non- | 4 | On treatment | 3.49/N | 5.65/Y | No Data |
| | response | | | | | |
| | week 24 | | | | | |
| 10 | Non- | 1 | On treatment | 2.87/Y | No Data | No Data |
| | response | | | | | |
| | week 12 | | | | | |

TABLE 5
12 MIU/Day Treatment Group

| | Previous | | | 4 Week | 12 Week | 24 Week |
|---|---|---|---|---|---|---|
| Subject | Therapy | Genotype | Outcome | Data/RVR | Data/EVR | Data/VN |
| 1 | Non- | 1 | Premature | 2.61/Y | No Data | No Data |
| | response | | discontinuation | | | |
| | week 24 | | week 9 | | | |
| 2 | Relapse | 1 | Non- | 2.12/N | 2.18/Y | 2.18/N |
| | | | response | | | |
| | | | week 24 | | | |
| 3 | Non- | 1 | Non- | 2.53/Y | 2.53/Y | No Data/N |
| | response | | response | | | |
| | week 24 | | week 24 | | | |
| 4 | Non- | 1 | Completed | 2.5/N | 2.88/Y | 4.95/Y |
| | response | | week 48 | | | |
| | week 12 | | | | | |
| 5 | HCV RNA | 1 | On treatment | 3.4/Y | 3.4/Y | 3.4/Y |
| | positive at | | | | | |
| | week 48, | | | | | |
| | viral | | | | | |
| | breakthrough | | | | | |
| 6 | Non- | 1 | Premature | No Data | No Data | No Data |
| | response | | discontinuation | | | |
| | week 12 | | week 3 | | | |
| 7 | Relapse | 1 | On treatment | 2.46/Y | 4.53/Y | No Data |
| 8 | Non- | 1 | Premature | 3.18/Y | 5.25/Y | No Data |
| | response | | discontinuation | | | |
| | week 24 | | week 21 | | | |
| 9 | Non- | 4 | On treatment | 2.68/N | No Data | No Data |
| | response | | | | | |
| | week 24 | | | | | |
| 10 | Relapse | 4 | On treatment | 2.58/Y | No Data | No Data |

### 3. Discussion of Data:

Early virologic response (EVR) is defined as at least a 2 log reduction in viral load by week 12 or viral negativity in patients with low initial viral loads. EVR is also associated strongly with ultimate clinical success but not as strongly as RVR. In the SCIN-C trial, 4 of 8 patients with measured viral data (50%) achieved EVR in the 6 MIU/day treatment group and 3 of 6 patients with measured viral data (50%) achieved EVR in the 9 MIU/day treatment group. In the highest dose groups (12 MIU/day), all 6 subjects (100%) of subjects who reached 12 weeks showed EVR.

Viral negativity (VN) at week 24 is a continuation requirement for the SCIN-C protocol. Patients who were not viral negative at week 24 were discontinued from the study. In the 6 MIU/day treatment group, 1 of 8 (12.5%) subjects who had 24 week data was viral negative while 2 subjects are still on treatment. In the intermediate dose of 9 MIU/ml, 2 of 8 (25%) subjects who had 24 week measurements were viral negative and 3 subjects are still on treatment. In the 12 MIU/day treatment group, 2 subjects had achieved VN at week 24, 2 subjects were viral positive at week 24 and 3 subjects remained on therapy.

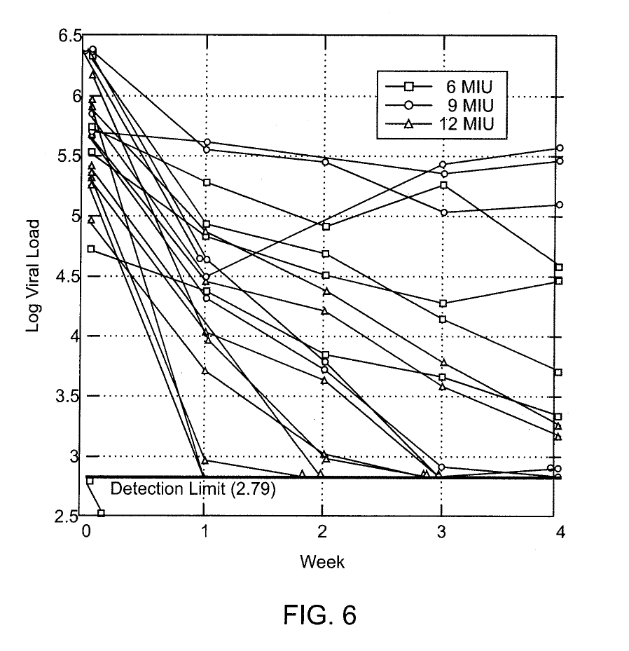

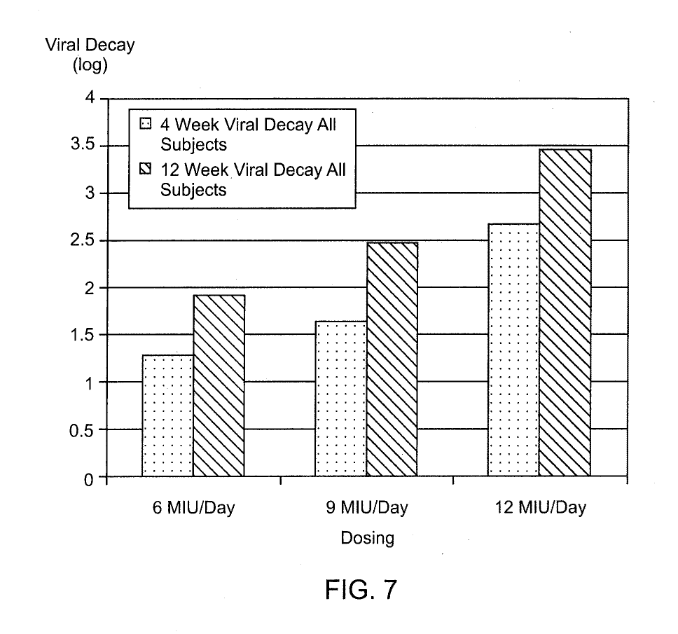

Viral decay data at the four week time point is shown in FIG. 6. As shown by the curves in this graph, at four weeks there is a significant difference between the doses. This is shown more clearly by FIG. 7, which shows viral decay by dosing (all patients).

The data provided herein shows that continuous dosing of interferon-α via subcutaneous infusion using an insulin pump with oral weight based ribavirin is both safe and effective and for the first time shows that by controlling blood levels of interferon-α we can get dose dependent viral kinetics. While this data demonstrates the efficacy of the disclosed methods in chronic hepatitis C treatment experienced patients, those of skill in the art understand that these methods are useful with hepatitis C treatment naive patients as well in view of for example, the importance of implementing regimens observed to result in a higher rate of therapeutic success (rather than, for example, adopting conventional therapeutic regimens observed to have higher rates of failure).

### II. Additional Analysis of Clinical Trial Data

As noted above, for the clinical trial, 30 HCV genotype 1 (n=24) and 4 (n=6) patients were randomized in a 1:1:1 ratio to receive 6, 9 or 12 MIU IFN alfa-2b daily by continuous subcutaneous administration using an insulin pump (Medtronic MiniMed 508) for 48 weeks. All patients in the trial received weight-based ribavirin (1000-1600 mg). HCV RNA levels, serum IFN-alfa levels, serum markers of immune activation (neopterin, 2,5-oligoadenylate synthetase [OAS], beta 2-microglobulin), in vitro T cell proliferation and IFN-gamma production were analyzed. Blood samples were collected at T=0, 4, 8, 12, 24, 48, 72, 96 hrs, week 1, 2, 3, 4 and 24 weeks post-treatment. The clinical trial was used to assess the safety and tolerability and to study viral kinetics in patients who had previously failed therapy (non-response: n=20; relapse: n=7; or viral breakthrough: n=3). In the 6, 9, and 12 MIU group cirrhosis was present in 3, 3, and 7 patients respectively. Virological responses are shown in Table 6 below. At week 4, a mean HCV RNA decline of 1.19 (95% CI 0.55-1.83), 1.21 (95% CI 0.38-2.04) and 2.67 (95% CI 2.38-2.97) log₁₀ IU/ml was found with 6, 9, and 12MIU IFN-α/day, respectively (12MIU vs. 9MIU/6MIU, p<0.0001). Out of the 20 previous non-responders 9 became HCV RNA negative by PCR during therapy and 3 achieved SVR (2 received 12 MIU/day and 1 received 9 MIU/day). Based on HCV RNA load at week 4, we identified n=13 responders (>2 log drop), n=10 intermediate responders (1-2 log drop) and n=5 nonresponders (<1 log drop). A typical biphasic viral decline was seen in responders. All patients achieving sustained virological response after 48 weeks of therapy (n=5) had >2 log drop of HCV RNA at week 4. IFN-α levels increased dose-dependently, reaching peak-levels between 48 hrs and week 1 followed by steady-state. Responders achieved higher IFN-α levels than nonresponders (mean 304.0 vs 160.2 pg/ml at week 4). Neopterin increased equally among all patients between 48 and 96 hrs, with higher steady-state levels in patients receiving 12MIU/day. Beta 2-microglobulin increased moderately in all patients; higher baseline levels were seen in responders (mean 16.9 vs 13.4 ug/ml). 2,5-OAS levels peaked between 24 and 96 hrs followed by slow decline, without differences in responders and nonresponders. Baseline T cell proliferation was strongly reduced when cultured in vitro with IFN-alfa in most patients, suggesting responsiveness to IFN-α irrespective of treatment outcome. However, desensitization of the cells for IFN-alfa with regard to T cell proliferation was seen especially in nonresponders at T=24 hrs. Baseline IFN-gamma production was variable between patients when cultured in vitro with IFN-alfa. Unresponsiveness of IFN-gamma production when cultured in vitro with IFN-alfa at T=0 and T=24 hrs was seen in the limited group of nonresponders.

AEs were mostly mild to moderate and were typical of IFN-α therapy but 5 patients developed irritation and/or abscesses at the injection site. Six serious adverse events (SAEs) were reported in 5 subjects, this led to permanent discontinuation in 3 subjects. All SAEs were consistent with high dose IFN-α therapy. Of the discontinuations due to SAEs, 2 subjects received the 12 MIU/day and 1 patient received the 9 MIU/day dose

The clinical trial data shows that a strong HCV RNA decline at week 4 can be induced by high dose continuous IFN-α therapy in patients who failed previous PegIFN-α/RBV therapy. Serum interferon-α levels, but no other immune activation markers, predict response. Consequently, the trial shows that doses of IFN-α can be delivered safely using continuous pump therapy in this difficult-to-treat population. Typical IFN-α-related

AEs appeared dose-dependent. In the intention-to-treat analysis SVR rate was 20% (6/30). In the per-protocol analysis SVR rate was 25% (6/24) of which 4 of the 6 in the high-dose arm reached SVR. With the successful management of side effects, continuous delivery of IFN-α can show significant clinical benefits. Interestingly, in vitro T cell and IFN-gamma proliferation before and shortly after start of therapy may identify patients unlikely to respond.

TABLE 6
Virological response: (undetectable HCV RNA by COBAS ®
Ampliprep/COBAS ® TaqMan ® HCV test, LLD <15 IU/mL).

| | | | | | | Subjects |
|---|---|---|---|---|---|---|
| Treatment | | | | | | Completing |
| Group | Week 4 | Week 12 | Week 24 | Week 48 | Week FU 24 | Protocol |
| 6 mu/day | 1/10 | 1/10 | 2/10 | 2/10 | 1/10 | 10/10 |
| 9 mu/day | 0/10 | 2/10 | 5/10 | 5/10 | 1/10 | 8/10 |
| 12 mu/day | 0/10 | 4/10 | 5/10 | 4/10 | 4/10 | 6/10 |

As shown for example by the data disclosed in this Example and the associated Figures, delivering concentrations of interferon-α following the therapeutic regimens disclosed herein leads to concentrations of interferon-α that are sustained in vivo and that these sustained in vivo concentrations of interferon-α can be used to eliminate HCV in a greater number of infected individuals than is possible following conventional therapeutic regimens. In particular, SVR was achieved in patients in each of the groups that received either 6, 9 or 12 MIU IFN alfa-2b daily by continuous subcutaneous administration for 48 weeks. Without being bound by a specific scientific theory, the surprising response observed in patients refractory to conventional therapy may result from interferon-α having a efficacy threshold that is: (1) met in only about 50% of patients treated according to conventional therapeutic regimens (perhaps due in part to different rates of exogenous interferon-α metabolism/clearance in different individuals); and (2) met in a greater number of patients when administered via a continuous infusion apparatus so as to maintain circulating levels of interferon-α in the serum of the patient above a steady state concentration (e.g. at least 100-700 pg/mL) for a sustained period of time (e.g. at least 1 to 48 weeks).

As disclosed herein, the group of patients receiving 12 MIU IFN alfa-2b daily by continuous subcutaneous administration for 48 weeks exhibited the best outcomes in response to this therapeutic regimen (e.g. SVR), followed by the group of patients receiving 9 MIU IFN-α, and then the group of patients receiving 6 MIU IFN-α. These results provide evidence that the dose of IFN-α administered to a patient is tied to the outcome (e.g. SVR), an observation that is further consistent with the observation that, in the clinical trial, responders achieved higher IFN-α levels than nonresponders (mean 304.0 vs 160.2 pg/ml at week 4 respectively). At the same time, the data from the clinical trial suggests that the administration of interferon-α in this manner can reduce the dose dependent adverse side effects that typically occur with the administration of these doses of interferon-α following conventional therapeutic regimens. Without being bound by a specific scientific theory, it is believed that a dose of interferon-α administered in this manner does not produce the same degree of adverse side effects typically experienced with a dose of interferon-α administered following conventional IFN-α based HCV therapies because the continuous administration of this therapeutic molecule can avoid the very high serum concentrations of interferon-α and continual fluctuations in serum levels of this therapeutic molecule that can occur with conventional HCV therapies and which are believed to contribute to the severity of adverse reactions and/or the general discomfort that can occur with such therapies (e.g. weekly boluses of interferon, daily boluses of interferon-α etc.).

The data from the clinical trial shows that SVR can be attained in patients refractory to conventional IFN-α/ribavirin HCV therapy by administering ribavirin in combination with 6 MIU IFN-α/day infused by continuous subcutaneous administration for 48 weeks. The data from the clinical trial further shows that serum interferon-α levels are predictive of a patient's response. As shown in FIG. 1A, over a period of four weeks, patients receiving 6 MIU IFN-α/day by continuous infusion attained a mean serum concentrations above 100 pg/mL and that these mean serum interferon-α levels are typically above 200 pg/mL Without being bound by a specific scientific theory, data from the clinical trial provides evidence that, in some refractory patients, a serum concentration above 100 pg/mL or 200 pg/mL is above a threshold IFN-α concentration that, when reached, induces and/or facilitates a patient's sustained response to a therapeutic regimen.

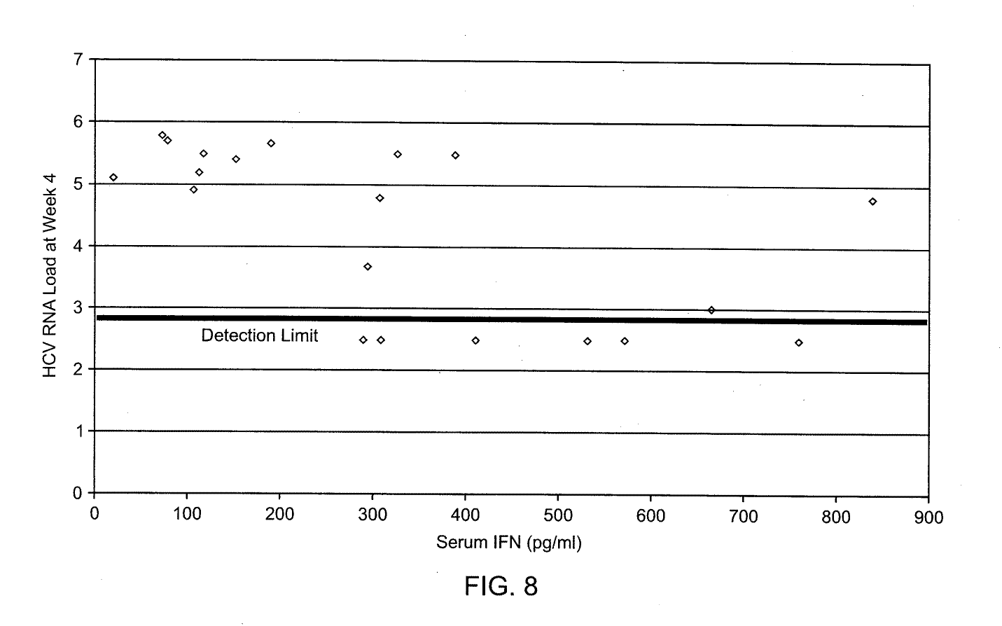

The data from the clinical trial shows that SVR can be attained in patients refractory to conventional IFN-α/ribavirin HCV therapy by administering ribavirin in combination with 9 MIU IFN-α/day infused by continuous subcutaneous administration for 48 weeks. The data from the clinical trial further shows that serum levels of exogenous interferon-αare predictive of a patient's response. As shown in FIG. 1A, over a period of four weeks, patients receiving 9 MIU IFN-α/day by continuous infusion attained mean serum IFN-α concentrations above 200 pg/mL, typically above 300 pg/mL Similarly, the data shown in FIG. 8 shows that, at week four, there is a correlation between undetectable HCV levels and patients having mean serum IFN-α concentrations around or above 300 pg/mL Without being bound by a specific scientific theory, data from the clinical trial provides evidence that, in some refractory patients, a serum concentration above 200 pg/mL or 300 pg/mL is above a threshold IFN-α concentration that, when reached, induces and/or facilitates a patient's sustained response to a therapeutic regimen.

The data from the clinical trial shows that SVR can be attained in patients refractory to conventional IFN-α/ribavirin HCV therapy by administering ribavirin in combination with 12 MIU IFN-α/day infused by continuous subcutaneous administration for 48 weeks. The data from the clinical trial further shows that serum interferon-α levels are predictive of a patient's response. As shown in FIG. 1A, over a period of four weeks, patients receiving 12 MIU IFN-α/day by continuous infusion attained mean serum IFN-α concentrations above 300 pg/mL, typically above 400 pg/mL Similarly, the data shown in FIG. 8 shows that, at week four, there is a correlation between undetectable HCV levels and patients having mean serum IFN-α concentrations above 300, above 400 or above 500 pg/mL Without being bound by a specific scientific theory, data from the clinical trial provides evidence that, in some refractory patients, a serum concentration above 300 pg/mL or 400 pg/mL (or higher) is above a threshold IFN-α concentration that, when reached, induces and/or facilitates a patient's sustained response to a therapeutic regimen.

As noted above, the data from the clinical trial shows that SVR can be attained in patients refractory to conventional IFN-α/ribavirin HCV therapy. Consequently, embodiments of the invention address a long-felt but unresolved need, specifically the need to eliminate HCV in a greater number of infected individuals than is possible using conventional therapeutic regimens. In addition, while the clinical trial focused on patients refractory to conventional IFN-α/ribavirin HCV therapy, those of skill in this art understand that embodiments of the invention are useful for treatment naive patients as well. For example by using embodiments of the invention to treat patients who have not experienced any prior therapeutic regimen, a group of individuals within the about 50% of patients observed to fail to respond to conventional therapy will be cured without having to experience 48 weeks of a failing conventional IFN-α/ribavirin therapeutic regimen (and the expense and side effects etc. associated with such conventional therapeutic regimens).

### Example 3

### Personalized Therapeutic Regimens

In typical embodiments of the invention, therapeutic protocols following parameters disclosed herein disclosed herein can be tailored to take into account patient specific factors that can influence a patients' response to treatment such as the HCV genotype(s) infecting the patient, and/or a patient's weight, treatment history, health status, individual rate of exogenous interferon-α clearance, and the like. In one illustrative embodiment of the invention relating to patient specific therapeutic regimens, a patient is administered interferon-α following a first therapeutic regimen that endeavors to produce mean or median circulating levels of interferon-α that fall within a target range, for example 100-200 pg/mL (or 150-250 pg/mL), 200-300 pg/mL (or 250-350 pg/mL), 300-400 pg/mL (or 350-450 pg/mL) up to 700 pg/mL, etc. Pharmacokinetic and/or pharmacodynamic parameters can then be obtained from the patient so as to observe a patient-specific response to this first therapeutic regimen (e.g. actual concentration of interferon-α in the blood of that specific patient that results from the first therapeutic regimen, the concentration of hepatitis C virus present in the patient etc.). Such empirical observations consider patient specific factors that influence a patients' response to treatment, for example a patient's unique rate of exogenous interferon-α metabolism/clearance (a factor which, for example, can influence the serum concentrations of interferon-α that are attained in a patient in response to a dose of interferon-α), and consequently can be used to design a patient-specific therapeutic regimen, for example one that modulates the concentration of interferon-α in the blood of the patient (e.g. so as to increase serum interferon-α levels as compared to the first therapeutic regimen administered to the patient).

Embodiments of the invention include personalized therapeutic regimens designed to produce a sustained virological response while simultaneously reducing or avoiding one or more of the adverse side effects that are observed to arise with lengthy treatment regimens comprising doses of interferon-α and ribavirin. As noted in the following paragraphs, embodiments of the invention consider factors such as: indicators of the patient's overall physiological health (e.g. Body Mass Index, the presence or absence of metabolic diseases such as diabetes etc.); and/or a patient's SNP genotype at a region of chromosome 19 and/or the genotype of the HCV virus and/or a patient's rate of exogenous interferon-α metabolism and/or the extent of an individual patient's desensitization of their T cells (with regard to T cell proliferation) in response to interferon-α etc. in order to design an personalized therapeutic regimen that that comprises administering effective amounts of interferon-α and ribavirin for a period of time sufficient to attain a sustained virological response. Personalized therapeutic regimens include those designed to avoid administering amounts of interferon-α and ribavirin that are greater than the critical amounts required to attain sustained virological response and/or avoid administering interferon-α and ribavirin for a period of time longer than the critical period required to attain sustained virological response. In this way, personalized therapeutic regimens can effectively treat patients while simultaneously reducing or avoiding the occurrence of one or more of the adverse side effects that are observed to arise in treatment regimens comprising doses of interferon-αand ribavirin.

In one illustrative embodiment, one or more patient SNP genotypes on chromosome 19 is determined. Because these SNP genotypes predict both treatment-induced viral clearance as well as the speed of a patient's response to treatment, this genotype information can be used to design personalized therapeutic regimens that include doses of interferon-α and ribavirin sufficient to attain sustained virological response yet avoid administering interferon-α and ribavirin for a period of time longer than the time period required to attain sustained virological response (i.e. so as to reduce the occurrence of adverse side effects). In addition to observing the sequences of single SNPs, certain embodiments of the invention observe the sequence of multiple SNPs, for example a group of SNPs within a haplotype block (i.e. SNPs close enough to one another on chromosome 19 to be inherited together).

Table 8 below includes a number of illustrative SNP genotypes identified as predictive of treatment induced viral clearance and/or the speed of a patent's response to therapeutic regimens comprising interferon-α and ribavirin. In this context, an artisan can, for example, determine if a patient is of the: CC, CT or TT genotype of the SNP designated rs12979860; AA, AG or GG genotype of the SNP designated rs12980275; GG, GT or TT genotype of the SNP designated rs8099917; AA, AC or CC genotype of the SNP designated rs12972991; AA, AC or CC genotype of the SNP designated rs8109886; AA, AG or GG genotype of the SNP designated rs4803223; CC, CT or TT genotype of the SNP designated rs12980602; TT, TC or CC genotype of the SNP designated rs8105790; AA, AG or GG genotype of the SNP designated rs11881222; CC, CT or TT genotype of the SNP designated rs8103142; CC, CG or GG genotype of the SNP designated rs28416813; CC, CT or TT genotype of the SNP designated rs4803219; AA, AG or GG genotype of the SNP designated rs7248668; AA, AC or CC genotype of the SNP designated rs4803217; and/or CC, CT or TT genotype of the SNP designated rs581930. In typical embodiments, analysis to determine a person's SNP genotype can be performed for example by real-time polymerase chain reaction (RT-PCR); using Taqman custom designed SNP specific probes (Applied Biosystems), on an ABI HT-7900 instrument using commercially available reagents from Applied Biosystems.

Typical methods of the invention comprise determining one or more SNP genotypes of a patient infected with hepatitis C virus; and then using this information to administering interferon-α to the patient according to a personalized therapeutic regimen, wherein the personalized therapeutic regimen comprises administering interferon-α subcutaneously using a continuous infusion apparatus, wherein the interferon-α is administered to the patient using a therapeutic regimen sufficient to maintain circulating levels of the interferon-α in the serum of the patient above a steady state concentration and/or within a target range, for example above 100 pg/mL and/or between 100-200 pg/mL (or 150-250 pg/mL); above 200 pg/mL and/or between 200-300 pg/mL (or 250-350 pg/mL); above 300 pg/mL and/or between 300-400 pg/mL (or 350-450 pg/mL); above 300 pg/mL and/or between 300-400 pg/mL (or 350-450 pg/mL); above 400 pg/mL and/or between 400-500 pg/mL (or 450-550 pg/mL); above 500 pg/mL and/or between 500-600 pg/mL (or 550-650 pg/mL); above 600 pg/mL and/or between 600-700 pg/mL (or 650-750 pg/mL); above 700 pg/mL etc. In this embodiment, the personalized therapeutic regimen is designed to allow the administration of interferon-α in an amount and for a period of time designed to produce a sustained virological response while also reducing or avoiding the occurrence of one or more of the adverse side effects associated with conventional regimens used for the administration of interferon.

As noted above, SNP genotypes can be used to predict treatment induced viral clearance, a factor that is also associated with the dose of interferon-α administered to a patient. In this context, in one specific illustrative example, one or more SNP genotypes of a patient infected with hepatitis C virus is determined and then this genotype information is used to design a personalized therapeutic regimen that comprises administering interferon-α subcutaneously using a continuous infusion apparatus, wherein the interferon-α is administered to the patient using a therapeutic regimen sufficient to maintain circulating levels of the interferon-α in the serum of the patient above a steady state concentration and wherein the dose of interferon-α administered to the patient is determined by the SNP genotype. For example, in some embodiments of the invention, the patient may have an SNP genotype observed to require a sustained dose of interferon-α of at least 6MIU per day to attain sustained virological response. In some embodiments of the invention, the patient may have an SNP genotype observed to require a sustained dose of interferon-α of at least 9MIU per day to attain sustained virological response. In some embodiments of the invention, the patient may have an SNP genotype observed to require a sustained dose of interferon-α of at least 12MIU per day to attain sustained virological response. Alternatively, in some embodiments of the invention, the patient may have an SNP genotype observed to require a sustained dose of interferon-α that is less than 12MIU per day to attain sustained virological response and consequently, a dose less than 12MIU per day is administered in order to avoid the occurrence of one or more of the adverse side effects that are observed to arise in treatment regimens comprising doses of interferon-α. Similarly, in some embodiments of the invention, the patient may have an SNP genotype observed to require a sustained dose of interferon-α that is less than 9MIU per day to attain sustained virological response and consequently, a dose less than 9MIU per day is administered in order to avoid the occurrence of one or more of the adverse side effects that are observed to arise in treatment regimens comprising doses of interferon-α. Similarly, in some embodiments of the invention, the patient may have an SNP genotype observed to require a sustained dose of interferon-α that is less than 6MIU per day to attain sustained virological response and consequently, a dose less than 6MIU per day is administered in order to avoid the occurrence of one or more of the adverse side effects that are observed to arise in treatment regimens comprising doses of interferon-α.

In a related illustrative example, one or more SNP genotypes of a patient infected with hepatitis C virus is determined and then this genotype information is used to design a personalized therapeutic regimen that comprises administering interferon-α subcutaneously using a continuous infusion apparatus, wherein the interferon-α is administered to the patient using a therapeutic regimen sufficient to maintain circulating levels of the interferon-α in the serum of the patient above a target or threshold steady state concentration and wherein the target or threshold steady state concentration of exogenous interferon-α in the patient is determined by the SNP genotype. Such embodiments of the invention are used to consider physiological process that may be specific for each patient, for example the rate at which a specific patient clears exogenous interferon-α administered according to one of the therapeutic regimens disclosed herein. In some embodiments of the invention, the patient may have an SNP genotype observed to require a target or threshold level of at least 100 pg/mL of exogenous interferon-α (i.e. exogenous interferon-α circulating in a patient's serum). In other embodiments of the invention, the patient may have an SNP genotype observed to require a target or threshold steady state concentration of exogenous interferon-α of at least 200 pg/mL. In other embodiments of the invention, the patient may have an SNP genotype observed to require a target or threshold steady state concentration of exogenous interferon-α of at least 300 pg/mL. In other embodiments of the invention, the patient may have an SNP genotype observed to require a target or threshold steady state concentration of exogenous interferon-α of at least 400 pg/mL. In other embodiments of the invention, the patient may have an SNP genotype observed to require a target or threshold steady state concentration of exogenous interferon-α of at least 500 pg/mL. In other embodiments of the invention, the patient may have an SNP genotype observed to require a target or threshold steady state concentration of exogenous interferon-α of at least 600 pg/mL. In other embodiments of the invention, the patient may have an SNP genotype observed to require a target or threshold steady state concentration of exogenous interferon-α of at least 700 pg/mL.

In another specific illustrative example, one or more SNP genotypes of a patient infected with hepatitis C virus is determined and then this genotype information is used to design a personalized therapeutic regimen that comprises administering interferon-α subcutaneously using a continuous infusion apparatus, wherein the interferon-α is administered to the patient using a therapeutic regimen sufficient to maintain circulating levels of the interferon-α in the serum of the patient above a steady state concentration and wherein the duration of the course of interferon-α administered to the patient is determined by the SNP genotype. For example, in some embodiments of the invention, the patient may have an SNP genotype observed to require a sustained dose of interferon-α for at least 48 weeks to attain sustained virological response. In other embodiments, the patient may have an SNP genotype observed to require a sustained dose of interferon-α for more time, for example at least 52, 56, 60, 64, 68 or 72 weeks to attain sustained virological response. In other embodiments, the patient may have an SNP genotype observed to require a sustained dose of interferon-α for less time to attain sustained virological response and this shortened period can be selected in order to shorten or diminish the side effects associated with interferon-α therapy. For example, in certain embodiments of the invention, the SNP genotype of the patient is one where a sustained virological response is typicality observed to be attained for example a period of time less than 48, 44, 36, 32 or 28 weeks.

It will be apparent to one skilled in the art that various combinations and/or modifications and variations can be made in such personalized therapeutic regimens depending upon the various physiological parameters observed in the patient. For instance, features illustrated or described as part of one embodiment can be used on another embodiment to yield a still further embodiment. For example, in certain embodiments of the invention, the SNP genotype is used to determine both the dose of interferon-α administered to the patient as well as the duration of interferon-α administration. In related embodiments of the invention, both the dose of interferon-α administered to the patient as well as the duration of interferon-α administration are determined using the SNP genotype in combination with additional factors such as the HCV genotype, the patient's prior treatment history (e.g. is a non-responder or relapser), the patient's body mass index and the like.

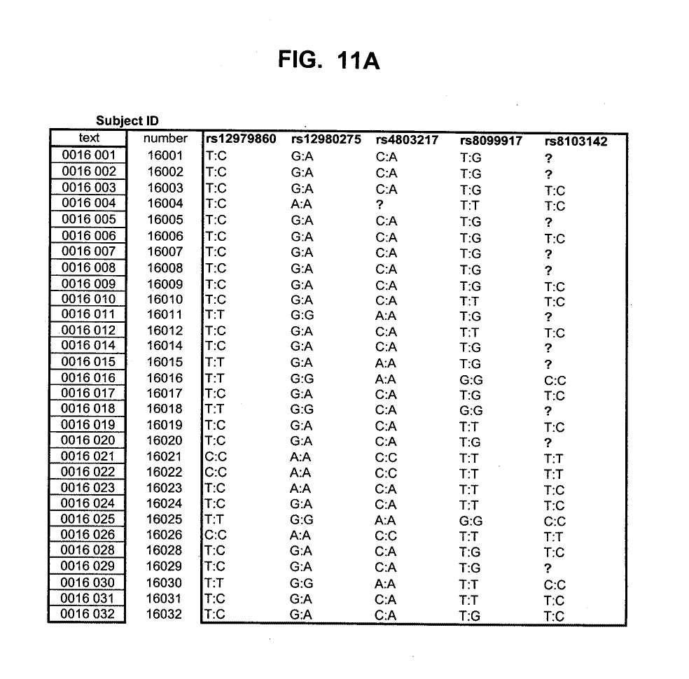

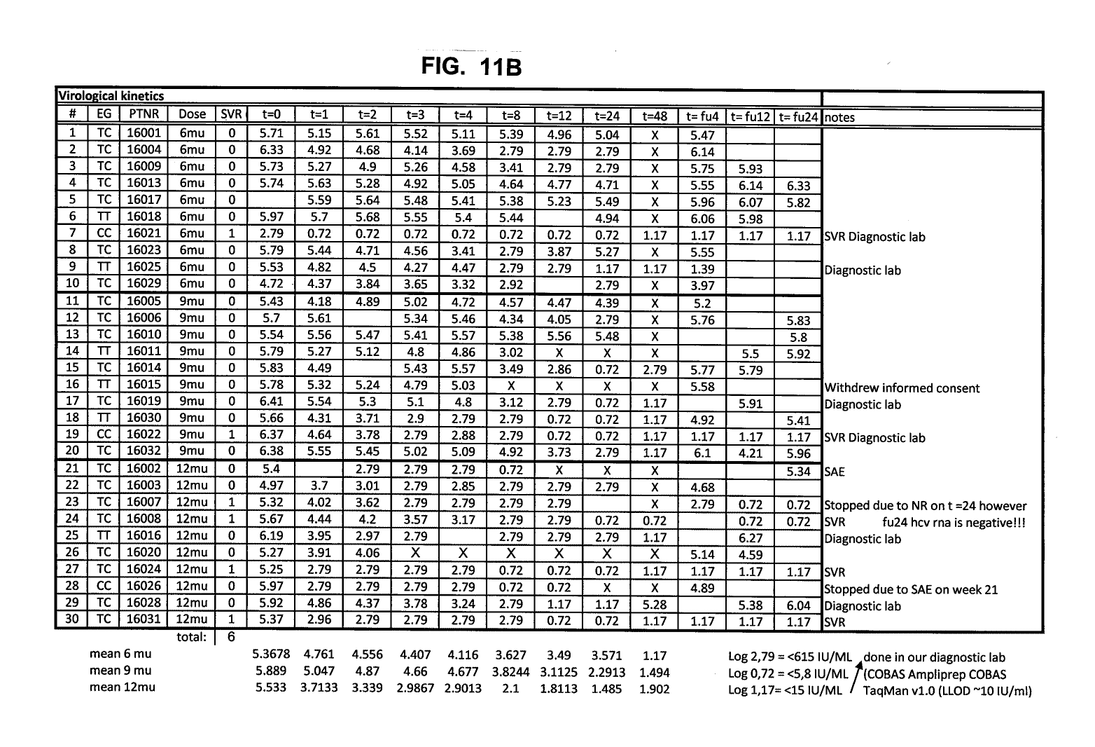

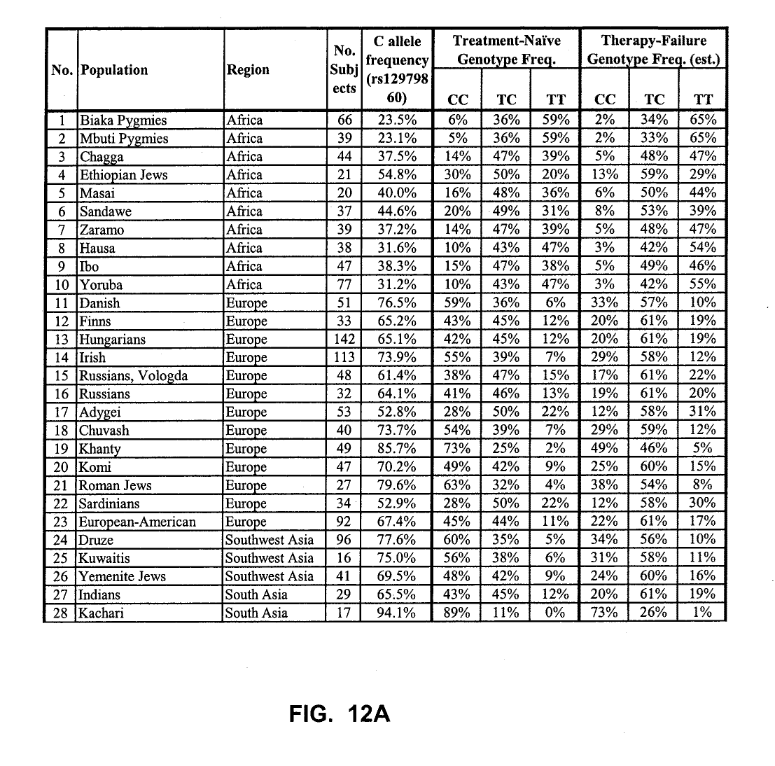

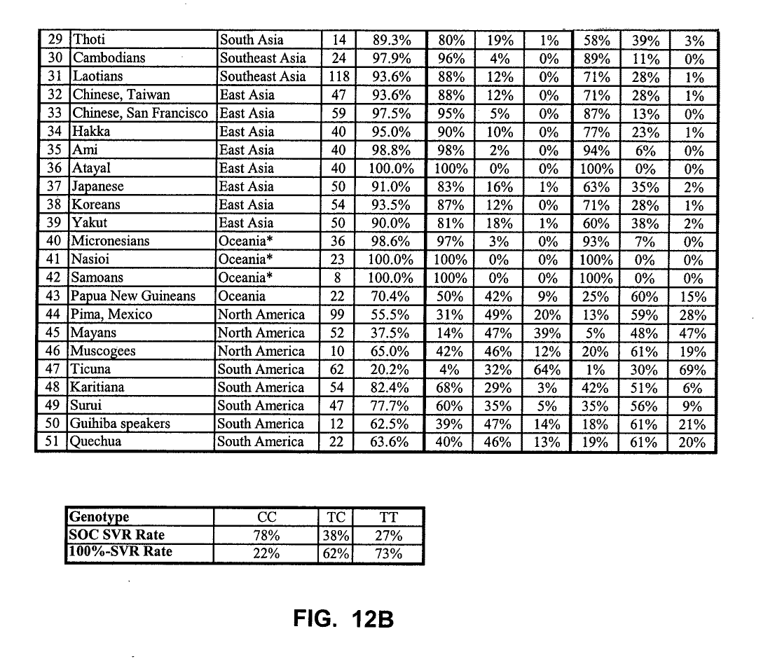

The sequence of SNP rs12979860 was examined in subjects from the SCIN-C study. As shown in the Table provided in FIG. 11B, this analysis shows that there were 3 subjects with the CC genotype, 21 subjects with the TC genotype, and 6 subjects with the TT genotype of SNP rs12979860. As shown in the Table provided in FIG. 11B, there is one subject in each of the 6 and 9 MIU/day interferon-α dosing arms who achieved SVR. Both of these subjects have the CC genotype for the IL28b gene SNP rs12979860. Publications in this technology teach that this is the “easy to treat” genotype (see, e.g. Ge et al., Nature 2009, 461(7262):399-401). As also shown in the Table provided in FIG. 11B, 4 subjects in the 12 MIU/day interferon-α dosing arm that achieved SVR. All of 4 of these genotypes are “TC”. It is our understanding that both the TC and TT genotypes are more difficult to treat than the CC genotype. The TC genotype appears to be more like the TT genotype than the CC genotype (see, e.g. Ge et al., Nature 2009, 461(7262):399-401). This data provides evidence that continuous interferon-α therapy at a dose of approximately 12 MIU/day is effective in curing patients with the rs12979860 SNP TC genotype including those that have failed to respond to a conventional therapeutic regimen. FIGS. 2-5 include patient data that is also shown in the SNP table in FIG. 11B. Comparisons of this data show that patients 1-10 in the graphs of 6 MIU data are patients 1-10 in this SNP table; patients 1-10 in the graphs of 9 MIU data are patients 11-20 in this SNP table, and patients 1-10 in the graphs of 12 MIU data are patients 21-30 in this SNP table. The Table shown in FIG. 12 provides an estimate of IL28B SNP rs12979860 genotype frequencies for 51 populations for both treatment-naïve and previous therapy failure patients. This estimate is based on disclosures known in the art including Ge et al., Nature 2009, 461(7262):399-401; and Thomas et al., Nature 2009, 461(7265):798-801.

As noted above, recent genome wide analysis studies (GWAS) of Hepatitis C patients have shown that in patients naïve to interferon, a single nucleotide polymorphism in the IL28B region can predict response to interferon/ribavirin therapy. Here we report the first analysis of IL28B SNP sequences in patients infected with HCV that are observed to be refractory to conventional HCV therapy. One analysis involves the SNP rs12979860. As expected from genome wide analysis studies and the population prevalence of the C allele, most patients in the study were discordant (CT) with 3 subjects being CC and 6 subjects being TT. In this context, this study shows that there were differences among the rs12979860 CC, CT and TT groups with respect to 4 week viral decay independent of dose. CC subjects had an average of 2.9 log drop at 4 weeks, while CT subjects showed a 1.65 log reduction and TT subject showed a 1.25 log reduction. Of the 5 evaluable TT subjects however, only two subjects showed more than 1 log reduction at 4 weeks. More importantly, in the high dose group at 12 MIU/day there were no virological failures (<2 log reduction at 4 weeks) in any IL28B cohort. This provides evidence that higher doses of IFN can potentially overcome the innate lack of interferon sensitivity that the IL28B SNP data suggests for treatment naïve patients.

Table 7 below shows the breakdown of subjects, IL28B SNP rs12979860 status, dose and viral decay rates. During the study, 6 subjects achieved SVR, 2 in the CC group (one in each of the 6 and 9 MIU/day dosing) and 4 in the high dose CT group. The CC subject in the high dose route was viral negative at 18 weeks and withdrew from the study at week 21 with subsequent viral breakthrough. These results strongly suggest that IL28B status can be a strong predictor of both viral decay rates and subsequent SVR. The results also strongly support the concept that continuous delivery of interferon in previous therapy failures can be a successful treatment strategy, especially those with CT genotypes.

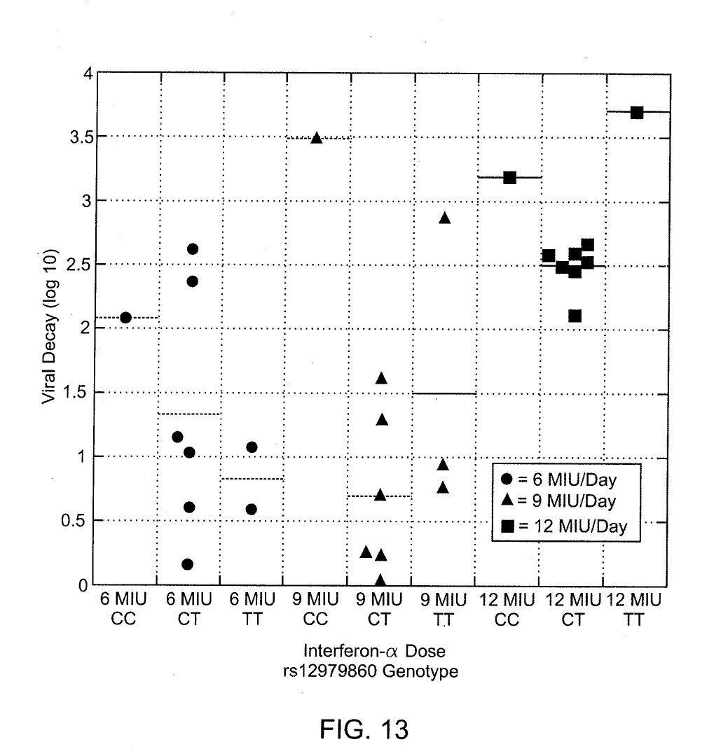

FIG. 13 provides a graph showing patient viral decay data in the context of both the dose of interferon administered the patients in the study as well as sequence information from the IL28B SNP designated rs12979860.

TABLE 7

| | IL28 B CC | IL28 B CT | IL28B TT |
|---|---|---|---|
| 6 MIU SVR/N | 1/1 | 0/6 | 0/2 |
| 6 MIU Viral | 2.07 | 1.36 | 0.81 |
| Decay Mean | | | |
| 6 MIU Range | 2.07/2.07 | 2.64/0.15 | 1.06/.81 |
| Hi/Low | | | |
| 9 MIU SVR/N | 1/1 | 0/6 | 0/3 |
| 9 MIU Viral | 3.49 | 0.69 | 1.51 |
| Decay Mean | | | |
| 9MIU Viral | 3.49/3.49 | 1.61/0.03 | 2.87/.75 |
| Decay Hi/Lo | | | |
| 12 MIU SVR/N | 0/1 | 4/7 | 0/1 |
| 12 MIU Decay | 3.18 | 2.49 | 3.4 |
| 12 MIU Hi/Lo | 3.18/3/18 | 2.68/2.12 | 3.7/3.7 |

### Single Nucleotide Polymorphisms

A number of different SNPs that are predictive of factors associated with HCV infection including treatment induced viral clearance and the speed of a patients response to interferon-α have been identified (see, e.g. Ge et al., Nature 2009, 461(7262):399-401; Tanaka et al., Nat. Genet. 2009 41(10):1105-9; Thomas et al., Nature 2009, 461(7265):798-801; Rauch et al., Gastroenterology 2010 138(4):1338-45; and McCarthy et al., Gastroenterology. 2010 138(7):2307-14, the contents of which are incorporated by reference herein). In this context, databases such as the Entrez Global Query Cross-Database Search System provide search engines that allow users to search databases at the National Center for Biotechnology Information (NCBI) website. Those of skill in this are aware, for example, that the Entrez SNP database provides a library of single nucleotide polymorphisms such as those disclosed in Ge et al., Nature. 2009; 461(7262): 399-401. In this database, the sequences of various polymorphism are cataloged with a SNP designation (e.g. rs12979860). Illustrative SNP sequences obtained using such SNP designations (e.g. rs12979860) as a query are provided in Table 8. In Table 8, the polymorphic nucleotide in these SNP sequences is bracketed (nucleotide position 27).

TABLE 8
ILLUSTRATIVE SINGLE NUCLEOTIDE POLYMORPHISMS IN CHROMOSOME 19

| | |
|---|---|
| rs12979860: | |
| CTGAACCAGGGAGCTCCCCGAAGGCG[C/T]GAACCAGGGTTGAATTGCACTCCGC | (SEQ ID NO: 1) |
| rs12980275: | |
| CTGAGAGAAGTCAAATTCCTAGAAAC[A/G]GACGTGTCTAAATATTTGCCGGGGT | (SEQ ID NO: 2) |
| rs8099917: | |
| CTTTTGTTTTCCTTTCTGTGAGCAAT[G/T]TCACCCAAATTGGAACCATGCTGTA | (SEQ ID NO: 3) |
| rs12972991 | |
| AGAACAAATGCTGTATGATTCCCCCT[A/C]CATGAGGTGCTGAGAGAAGTCAAAT | (SEQ ID NO: 4) |
| rs8109886 | |
| TATTCATTTTTCCAACAAGCATCCTG[A/C]CCCAGGTCGCTCTGTCTGTCTCAAT | (SEQ ID NO: 5) |
| rs4803223 | |
| CCTAAATATGATTTCCTAAATCATAC[A/G]GACATATTTCCTTGGGAGCTATACA | (SEQ ID NO: 6) |
| rs12980602: | |
| TCATATAACAATATGAAAGCCAGAGA[C/T]AGCTCGTCTGAGACACAGATGAACA | (SEQ ID NO: 7) |
| rs8103142: | |
| TCCTGGGGAAGAGGCGGGAGCGGCAC[C/T]TGCAGTCCTTCAGCAGAAGCGACTC | (SEQ ID NO: 8) |
| rs28416813: | |
| CAGAGAGAAAGGGAGCTGAGGGAATG[C/G]AGAGGCTGCCCACTGAGGGCAGGGG | (SEQ ID NO: 9) |
| rs4803219: | |
| CTGAGCTCCATGGGGCAGCTTTTATC[C/T]CTGACAGAAGGGCAGTCCCAGCTGA | (SEQ ID NO: 10) |
| rs4803217: | |
| TAGCGACTGGGTGACAATAAATTAAG[A/C]CAAGTGGCTAATTTATAAATAAAAT | (SEQ ID NO: 11) |
| rs581930: | |
| CTGTGGAGCACAGAACTGCCAGGAAC[C/T]AGGGCCCCTGGATGACTGAGTGGGG | (SEQ ID NO: 12) |
| rs8105790: | |
| CTTCCTGACATCACTCCAATGTCCTG[C/T]TTCTGTGGTTACATCTTCCGCTAAT | (SEQ ID NO: 13) |
| rs11881222: | |
| AGAGGGCACAGCCAGTGTGGTCAGGT[A/G]GGAGCAGAGGGAAGGGGTAGCAGGT | (SEQ ID NO: 14) |
| rs7248668: | |
| CATGGTCTCAGTCTGTAGCCCAAGCT[A/G]GAGCATAGTAGTGGCACAATCGCCA | (SEQ ID NO: 15) |

1. A method of administering interferon-α to a patient infected with hepatitis C virus, the method comprising:
administering interferon-α to the patient using a continuous infusion apparatus, wherein the interferon-α is administered to the patient using a therapeutic regimen sufficient to maintain circulating levels of the interferon-α in the serum of the patient above a mean steady state concentration of 100 pg/mL for at least 1 week to at least 48 weeks.

2. The method of claim 1, wherein the therapeutic regimen is sufficient to maintain circulating levels of the interferon-α in the patient above a mean steady state concentration of at least 200, 300, 400, 500, 600 or 700 pg/mL.

3. The method of claim 1, wherein the method further comprises determining a polynucleotide sequence of the patient, wherein the polynucleotide sequence comprises a single nucleotide polymorphism (SNP) designated rs12979860, rs12980275, rs8099917, rs12972991, rs8109886, rs4803223, rs8103142, rs28416813, rs4803219, rs4803217, rs581930, rs8105790, rs11881222, rs7248668 or rs12980602.

4. The method of claim 3, wherein the SNP is rs12979860 and the method comprises determining if the patient comprises a CC genotype, a TT genotype or a CT genotype.

5. The method of claim 3, further comprising using polynucleotide sequence information to determine or modulate a parameter of the therapeutic regimen, wherein the parameter comprises:
a duration of interferon-α administration; or
an interferon-α dose.

6. The method of claim 1, further comprising identifying the patient as a relapser or a non-responder.

7. The method of claim 1, further comprising identifying the hepatitis C virus as being a genotype 1 or a genotype 4 virus.

8. The method of claim 1, wherein the therapeutic regimen is administered for a duration of at least 5, 6, 7, 8, 12, 24, 36 or 48 weeks.

9. The method of claim 1, wherein the therapeutic regimen is sufficient to reduce levels of HCV in the patient by at least 2 logs (100-fold) or 3 logs (1000 fold) after 1, 2, 4, 8 10, 12, 14 or 16 weeks.

10. The method of claim 1, wherein the interferon-α is not conjugated to a polyol.

11. A method of administering an interferon-α to a patient infected with hepatitis C virus, the method comprising:
(a) administering a dose of interferon-α to the patient;
(b) observing a concentration of circulating interferon-α in serum of the patient that results from the dose of interferon-α;
(c) using the concentration of circulating interferon-α observed in step (b) to make a patient-specific therapeutic regimen, wherein the patient specific therapeutic regimen comprises administering interferon-α to the patient subcutaneously using a continuous infusion apparatus in an amount sufficient to maintain circulating levels of interferon-α in the serum of the patient above a steady state concentration of at least 100 pg/mL.

12. The method of claim 11, wherein the method further comprises:
determining a polynucleotide sequence of the patient, wherein the polynucleotide sequence comprises a single nucleotide polymorphism (SNP) designated rs12979860, rs12980275, rs8099917, rs12972991, rs8109886, rs4803223, rs8103142, rs28416813, rs4803219, rs4803217, rs581930, rs8105790, rs11881222, rs7248668 or rs12980602; and
using polynucleotide sequence information to determine or modulate a parameter of the patient-specific therapeutic regimen, wherein the parameter comprises:
a duration of interferon-α administration; or
an interferon-α dose.

13. The method of claim 11, further comprising:
identifying the patient as a relapser or a non-responder;
identifying the hepatitis C virus as being a genotype 1 or a genotype 4 virus;
observing in vitro proliferation of T cells from the patient in response to exposure to interferon-α;
administering interferon-α that is not conjugated to a polyol; or
administered interferon-α to the patient using a patient-specific therapeutic regimen sufficient to maintain circulating levels of interferon-α in the serum of the patient above a steady state concentration of at least 200, 300, 400, 500, 600 or 700 pg/mL for at least 1 week to at least 48 weeks.

14. The method of claim 11, wherein:
the patient-specific therapeutic regimen is administered for a duration of at least 5, 6, 7, 8, 12, 24, 36 or 48 weeks; or
the patient-specific therapeutic regimen is sufficient to reduce levels of HCV in the patient by at least 2 logs (100-fold) or 3 logs (1000 fold) after 1, 2, 4, 8 10, 12, 14 or 16 weeks.

15. A system for administering interferon-α to a patient having a hepatitis C infection, the system comprising:
a continuous infusion pump having a medication reservoir comprising interferon-α;
a processor operably connected to the continuous infusion pump and comprising a set of instructions that causes the continuous infusion pump to administer the interferon-α to the patient according to a therapeutic regimen comprising administering interferon-α to the patient subcutaneously; wherein the therapeutic regimen is sufficient to maintain circulating levels of interferon-α in the serum of the patient above a steady state concentration of at least 100 pg/mL for at least 1 week to at least 48 weeks.

16. The system of claim 15, wherein:
(a) the hepatitis C virus is a genotype 1 HCV;
(b) the hepatitis C virus is a genotype 4 HCV;
(c) the patient is identified as a relapser prior to administering the interferon-α;
(d) the patient is identified as a non-responder prior to administering the interferon-α;
(e) the therapeutic regimen is sufficient to maintain circulating levels of interferon-α in the patient above a concentration of at least 200, 300, 325, 350, 375, 400, 425, 450, 475, 500, 525, 550, 575, 600, 625, 650, 675 or 700 pg/mL;
(f) the therapeutic regimen is administered for a duration of at least 4, 8, 12, 24, 36 or 48 weeks;
(g) the therapeutic regimen is sufficient to reduce levels of HCV in the patient by at least 2 logs (100-fold) or 3 logs (1000 fold) after 1, 2, 4, 8 10, 12, 14 or 16 weeks; or
(h) the interferon-α is not conjugated to a polyol.

17. The system of claim 15, wherein:
a polynucleotide sequence of the patient using the system is determined, the polynucleotide sequence comprising a single nucleotide polymorphism (SNP) designated rs12979860, rs12980275, rs8099917, rs12972991, rs8109886, rs4803223, rs8103142, rs28416813, rs4803219, rs4803217, rs581930, rs8105790, rs11881222, rs7248668 or rs12980602; and
the processor in the system is used to modulate a parameter of the patient-specific therapeutic regimen using determined polynucleotide sequence information, wherein the parameter comprises:
a duration of interferon-α administration; or
an interferon-α dose.

18. The system for administering interferon-α of claim 15, wherein the system for administering interferon-α is coupled to an electronic system for managing medical data on an electronic communication network, the electronic system comprising:
at least one electronic server connectable for communication on the communication network, the at least one electronic server being configured for:
receiving information on a first physiological parameter observed in a patient;
setting a first dosage of the interferon-α for infusion by the continuous infusion pump, based on the first physiological parameter;
receiving second physiological parameter information of the patient indicative of a response of the patient to the interferon-α of the first dosage; and
setting a second dosage of the interferon-α for infusion by the continuous infusion pump, based on the second physiological parameter.

19. The system of claim 15, wherein the continuous infusion pump:
has dimensions smaller than 15×15 centimeters; or
is operably coupled to an interface that facilitates the patient's movements while using the continuous infusion pump, wherein the interface comprises a clip, a strap, a snap, a clamp or an adhesive strip.

20. Use of interferon-α in the manufacture of a composition for treating hepatitis C infection for use in a continuous infusion apparatus, wherein the interferon-α composition is manufactured to allow the continuous infusion apparatus to maintain mean circulating levels of interferon-α in serum of a patient above a steady state concentration of at least 100 pg/mL for at least 1 to at least 48 weeks when administered subcutaneously.
